(19) United States
(12) Patent Application Publication
(10) Pub. No.: US 2025/0311927 A1
(43) Pub. Date: Oct. 9, 2025

(54) PORTABLE HYPERSPECTRAL IMAGING DEVICE

(71) Applicant: Hypermed Imaging, Inc., Memphis, TN (US)

(72) Inventors: Mark Anthony Darty, Collierville, TN (US); Peter Martin Meenen, Cane Ridge, TN (US); James Douglas Fletcher, Bartlett, TN (US)

(21) Appl. No.: 18/955,675

(22) Filed: Nov. 21, 2024

Related U.S. Application Data
(63) Continuation of application No. 17/996,899, filed on Oct. 21, 2022, now abandoned.
(60) Provisional application No. 63/014,331, filed on Apr. 23, 2020.
(60) Provisional application No. 63/039,900, filed on Jun. 16, 2020.

(51) Int. Cl. A61B 5/00

(52) U.S. Cl. CPC A61B 5/0075; A61B 5/441; A61B 2562/0233

(57) ABSTRACT

Disclosed is a portable hyperspectral/multiple spectral imaging device. The imaging device has a chassis having a base face and an axis orthogonal to the base face. The chassis includes an inner perimeter wall extended substantially around the axis and enclosing an interior region of the chassis. The chassis also includes one or more outer walls extended at acute angles with respect to the base face and arranged around the inner perimeter wall. One or more light sources are disposed on the outer walls. The imaging device further comprises a lens, an optical filter, and an optical detector disposed within the interior region. The imaging device further comprises a control system and a low-voltage power source.

### RELATED APPLICATIONS

This application claims priority to U.S. Provisional Patent Application Ser. No. 63/014,331, filed Apr. 23, 2020, and U.S. Provisional Patent Application Ser. No. 63/039,900, filed Jun. 16, 2020, the disclosure of which are hereby incorporated by reference in their entireties.

### TECHNICAL FIELD

The present disclosure relates to hyperspectral spectroscopy, and more particularly, to medical hyperspectral imaging devices for home healthcare use.

### BACKGROUND

Hyperspectral spectroscopy is an imaging technique that integrates multiple images of an object resolved at different spectral bands (e.g., ranges of wavelengths) into a single data structure, referred to as a three-dimensional hyperspectral data cube. Hyperspectral spectroscopy is often used to identify an individual component of a complex composition through the recognition of corresponding spectral signatures of the individual components in a particular hyperspectral data cube.

Hyperspectral spectroscopy has been used in a variety of applications, ranging from geological and agricultural surveying to military surveillance and industrial evaluation. Hyperspectral spectroscopy has also been used in medical applications to facilitate complex diagnosis and predict treatment outcomes. For example, medical hyperspectral imaging has been used to determine tissue oxygenation. Adequate tissue oxygenation is vital for restoration of tissue function and integrity. In wound healing, adequate tissue oxygenation can trigger healing responses and favorably influence the outcomes of other treatment modalities.

Conventional medical hyperspectral imaging devices are expensive and complex, practically restricting their use to a clinical setting. However, the strict adherence to traditional medical procedure codes by medical insurance companies, as well as the difficulty in obtaining new medical procedure codes, for reimbursement purposes renders even clinical use of these devices financially restrictive relative to alternative technologies that provide less and/or less accurate information to the clinician. For example, the use of pulse oximeters to measure tissue oxygenation is much less expensive than hyperspectral imaging, but does not provide spatial information and gives a less complete understanding of total hemoglobin levels, oxygen saturation, etc. Moreover, in many cases, regular monitoring of a medical condition, e.g., monthly, weekly, daily, or more frequently, results in better outcomes. However, regular visits to a clinic for hyperspectral monitoring is inconvenient and expensive. For example, daily monitoring of diabetic foot ulceration would allow a clinician to identify problematic ulcers and/or ineffective treatment thereof more quickly, avoiding otherwise unnecessary amputations and loss of life. However, this is impracticable given the current state of hyperspectral medical imaging.

The information disclosed in this Background section is provided for an understanding of the general background of the invention and is not an acknowledgement or suggestion that this information forms part of the prior art already known to a person skilled in the art.

### SUMMARY

Given the current state of the art, there remains a need for systems, methods and devices that facilitate medical hyperspectral imaging outside of a clinical setting, for example for home healthcare. Advantageously, the present disclosure provides systems, method, and devices that solve this and other needs. Specifically, in some aspects, the present disclosure provides inexpensive hyperspectral imaging devices with extremely low power requirements that facilitate patient self-monitoring.

Various implementations of systems, methods and devices within the scope of the appended claims each have several aspects, no single one of which is solely responsible for the desirable attributes described herein. Without limiting the scope of the appended claims, some prominent features are described herein.

In one aspect, the disclosure provides a device including a chassis having a first side facing an exterior, a second side facing away from the exterior, and an axis pointing from the second side to the first side. The chassis includes an inner perimeter wall extended substantially along the axis of the chassis from the second side toward the first side of the chassis, and one or more oblique outer walls extended at an acute angle with respect to the axis of the chassis from the second side toward the first side of the chassis. The device also includes a light source attached to or disposed at the oblique outer wall of the chassis. The light source is configured to illuminate a region of interest (ROI). The device also includes a lens attached to or disposed within the chassis. The lens is configured to collect light backscattered by the ROI. The device also includes an optical filter attached to or disposed within the chassis. The optical filter is configured to filter the light collected by the lens, where the optical filter includes a plurality of optical filter elements. Each respective optical filter element in the plurality of optical filter elements is in optical communication with the lens, allowing light of a corresponding spectral range in a plurality of spectral ranges to pass while blocking light of any other spectral range in the plurality of spectral ranges. The device also includes an optical detector attached to or disposed within the chassis. The optical detector is in optical communication with the optical filter and configured to resolve light filtered by the optical filter. The optical detector includes a plurality of optical detector elements. Each respective optical detector element in the plurality of optical detector elements is covered by a corresponding optical filter element in the plurality of optical filter elements. The device also includes a control system to control operation of the light source and the optical detector. The optical detector is surrounded by the inner perimeter wall of the chassis. The inner perimeter wall of the chassis has a height along the axis of the chassis to block stray light of the light source from reaching the plurality of optical detectors.

In another aspect, the disclosure provides a device that includes a chassis having a first side facing an exterior, a second side facing away from the exterior, and an axis pointing from the second side to the first side. The chassis includes an inner perimeter wall extended substantially along the axis of the chassis from the second side toward the first side of the chassis and an oblique outer wall extended at an acute angle with respect to the axis of the chassis from the second side toward the first side of the chassis. The device also includes a light source attached to or disposed at the oblique outer wall of the chassis. The light source is configured to illuminate a region of interest (ROI). The device also includes a lens attached to or disposed within the chassis. The lens is configured to collect light backscattered by the ROI. The device also includes an optical filter attached to or disposed within the chassis. The optical filter is in optical communication with the lens and configured to filter the light collected by the lens. The optical filter includes an array of filter regions, each filter region in the array of filter regions including a plurality of optical filter elements. Each respective optical filter element in the plurality of optical filter elements allows light of a corresponding spectral range in a plurality of spectral ranges to pass while blocking light of any other spectral range in the plurality of spectral ranges. The device also includes an optical detector attached to or disposed within the chassis. The optical detector is in optical communication with the optical filter and configured to resolve light filtered by the optical filter. The optical detector includes an array of detector regions, and each detector region in the array of detector regions includes a plurality of optical detector elements. Each respective optical detector element in the plurality of optical detector elements is covered by a corresponding optical filter element in the plurality of optical filter elements. The device also includes a control system to control operation of the light source and the optical detector. The optical detector is surrounded by the inner perimeter wall of the chassis. The inner perimeter wall of the chassis has a height along the axis of the chassis to block stray light of the light source from reaching the plurality of optical detectors.

In another aspect, the disclosure provides a device that includes a chassis having a first side facing an exterior, a second side facing away from the exterior, and an axis pointing from the second side to the first side. The chassis includes a perimeter wall extended substantially along the axis of the chassis from the second side toward the first side of the chassis and an oblique outer wall extended at an acute angle with respect to the axis of the chassis from the second side toward the first side of the chassis. The device also includes a light source including a plurality of light source elements attached to or disposed at the oblique outer wall of the chassis. The light source is configured to illuminate a region of interest (ROI). The device also includes an optical filter including a plurality of optical filter elements. Each respective optical filter element in the plurality of optical filter elements covers a corresponding light source element in the plurality of light source elements, and allows light of a corresponding spectral range in a plurality of spectral ranges to pass while blocking light of any other spectral range in the plurality of spectral ranges. The device also includes a lens attached to or disposed within the chassis and configured to collect light backscattered by the ROI. The device also includes an optical detector attached to or disposed within the chassis, in optical communication with the lens, and configured to resolve the light collected by the lens. The optical detector comprises an array of optical detector elements. The device also includes a control system to control operation of the light source and the optical detector. The optical detector is surrounded by the inner perimeter wall of the chassis. The inner perimeter wall of the chassis has a height along the axis of the chassis to block stray light of the plurality of light sources from reaching the array of optical detector elements.

In another aspect, the disclosure provides a device that includes a chassis. The device also includes a light source attached to or disposed at the chassis. The light source is configured to illuminate a region of interest (ROI). The device also includes a lens attached to or disposed within the chassis and configured to collect light backscattered by the ROI. The device also includes an optical filter attached to or disposed within the chassis. The optical filter is configured to filter the light collected by the lens. The optical filter includes a plurality of optical filter elements. Each respective optical filter element in the plurality of optical filter elements is in optical communication with the lens, allowing light of a corresponding spectral range in a plurality of spectral ranges to pass while blocking light of any other spectral range in the plurality of spectral ranges. The device also includes an optical detector attached to or disposed within the chassis and configured to resolve light filtered by the optical filter. The optical detector includes a plurality of optical detector elements, and each respective optical detector element in the plurality of optical detector elements is covered by a corresponding optical filter in the plurality of optical filters. The device also includes a control system to control operation of the light source and the optical detector. The device also includes a power source in electrical communication with the light source, the optical detector, and the control system. The power source has a nominal voltage of 10 volts or less and is configured to provide electrical power for operating the light source, the optical detector, and the control system.

In another aspect, the disclosure provides a device that includes a chassis. The device also includes a light source attached to or disposed at the chassis. The light source device is configured to illuminate a region of interest (ROI). The device also includes a lens attached to or disposed within the chassis. The lens is configured to collect light backscattered by the ROI. The device also includes an optical filter attached to or disposed within the chassis, in optical communication with the lens and configured to filter the light collected by the lens. The optical filter includes an array of filter regions. Each filter region in the array of filter regions includes a plurality of optical filter elements. Each respective optical filter element in the plurality of optical filter elements allows light of a corresponding spectral range in a plurality of spectral ranges to pass while blocking light of any other spectral range in the plurality of spectral ranges. The device also includes an optical detector attached to or disposed within the chassis, in optical communication with the optical filter and configured to resolve light filtered by the optical filter. The optical detector includes an array of detector regions. Each detector region in the array of detector regions includes a plurality of optical detector elements. Each respective optical detector element in the plurality of optical detector elements is covered by a corresponding optical filter element in the plurality of optical filter elements. The device also includes a control system to control operation of the light source and the optical detector. The device also includes a power source in electrical communication with the light source, the optical detector and the control system. The power source has a nominal voltage of 10 volts or less and is configured to provide electrical power for operating the light source, the optical detector and the control system.

In another aspect, the disclosure provides a device that includes a chassis. The device also includes a light source comprising a plurality of light source elements spatially attached to or disposed at the chassis and configured to illuminate a region of interest (ROI). The device also includes an optical filter including a plurality of optical filter elements. Each respective optical filter element in the plurality of optical filter elements covers a corresponding light source element in the plurality of light source elements. Each respective optical filter element allows light of a corresponding spectral range in a plurality of spectral ranges to pass while blocking light of any other spectral range in the plurality of spectral ranges. The device includes a lens attached to or disposed within the chassis. The lens is configured to collect light backscattered by the ROI. The device also includes an optical detector attached to or disposed within the chassis. The optical detector is in optical communication with the lens, and configured to resolve the light collected by the lens. The optical detector includes an array of optical detector elements, thereby generating an array of detector outputs for each pair of the light source element and the filter element. The device also includes a control system to control operation of the light source and the optical detector. The device also includes a power source in electrical communication with the light source, the optical detector and the control system, wherein the power source has a nominal voltage of 10 volts or less and is configured to provide electrical power for operating the light source, the optical detector and the control system.

In another aspect, the disclosure provides for a system including the device described herein, a first client device in a wireless communication with the device, and a server in a wireless communication with the device and the first client device. The server includes one or more central processing units, memory, and one or more programs. The one or more programs are stored in the memory and are configured to be executed by the one or more central processing units. The one or more programs including instructions for receiving, from the device, the hyperspectral data cube of detector outputs, forming a hyperspectral image using the hyperspectral data cube of detector outputs, and transmitting, to the first client device, the hyperspectral image.

Still further aspects of the present disclosure provide nontransitory computer-readable storage mediums storing one or more programs. The one or more programs comprises instructions, which when executed by a device comprising a processor and memory, cause the device to perform the methods or any one or more steps of the methods disclosed herein.

### BRIEF DESCRIPTION OF THE DRAWINGS

So that the present disclosure can be understood in greater detail, a more particular description may be had by reference to aspects of various implementations, some of which are illustrated in the appended drawings. The appended drawings, however, merely illustrate the more pertinent aspects of the present disclosure and are therefore not to be considered limiting, as the description may admit to other effective aspects and arrangements.

FIG. 1 is a schematic block diagram illustrating an example imaging device, in accordance with some embodiments of the present disclosure.

FIGS. 2A, 2B, and 2C illustrate several views of an example imaging module, in accordance with some embodiments of the present disclosure.

FIG. 3 illustrates use of an example imaging module and a handheld enclosure, in accordance with some embodiments of the present disclosure.

FIG. 4 illustrates use of an example imaging module and a wearable enclosure, in accordance with some embodiments of the present disclosure.

FIGS. 5A, 5B, 5C, 5D, 5E, 5F, 5G, and 5H illustrate several views of an example imaging device, in accordance with some embodiments of the present disclosure. FIGS. 5A, 5B, 5C and 5D illustrate several views of the internal architecture of the example device. FIGS. 5E and 5F illustrate several views of the exterior of the example device. FIG. 5G illustrates a cross-sectional view of the example device. FIG. 5H illustrates another cross-sectional view of an example device in accordance with some embodiments of the present disclosure.

FIGS. 6A and 6B illustrate several views of the internal architecture of an example imaging device, in accordance with some embodiments of the present disclosure.

FIG. 6C illustrates a cross-sectional view of an example imaging device illuminating a region of interest, in accordance with some embodiments of the present disclosure.

FIGS. 7A and 7B illustrate views of the internal architecture of an example device, in accordance with some embodiments of the present disclosure.

FIG. 7C illustrates the example device of FIGS. 7A and 7B in an example handheld housing, in accordance with some embodiments of the present disclosure.

FIG. 8 illustrates a block diagram of method 800 of collecting and generating hyperspectral image data, in accordance with some embodiments of the present disclosure.

FIG. 9 is a block diagram illustrating a distributed client-server system for processing hyperspectral images via a computer network, in accordance with some implementations.

In accordance with common practice the various features illustrated in the drawings may not be drawn to scale. The dimensions of various features may be arbitrarily expanded or reduced for clarity. In addition, some of the drawings may not depict all of the components of a given system, method or device. Like reference numerals may be used to denote like features throughout the specification and figures.

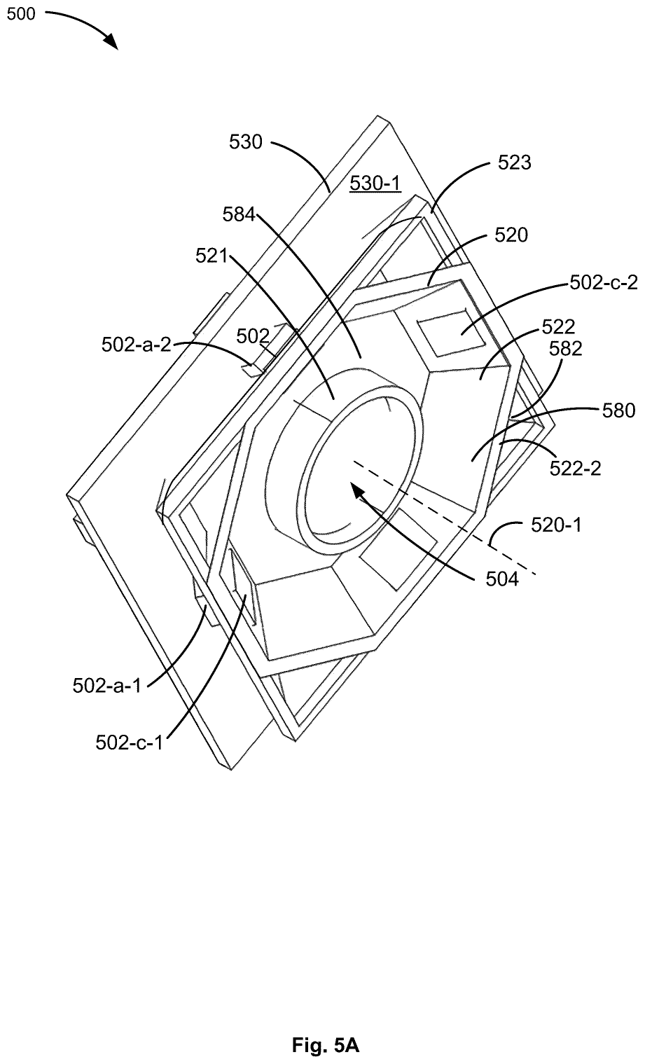

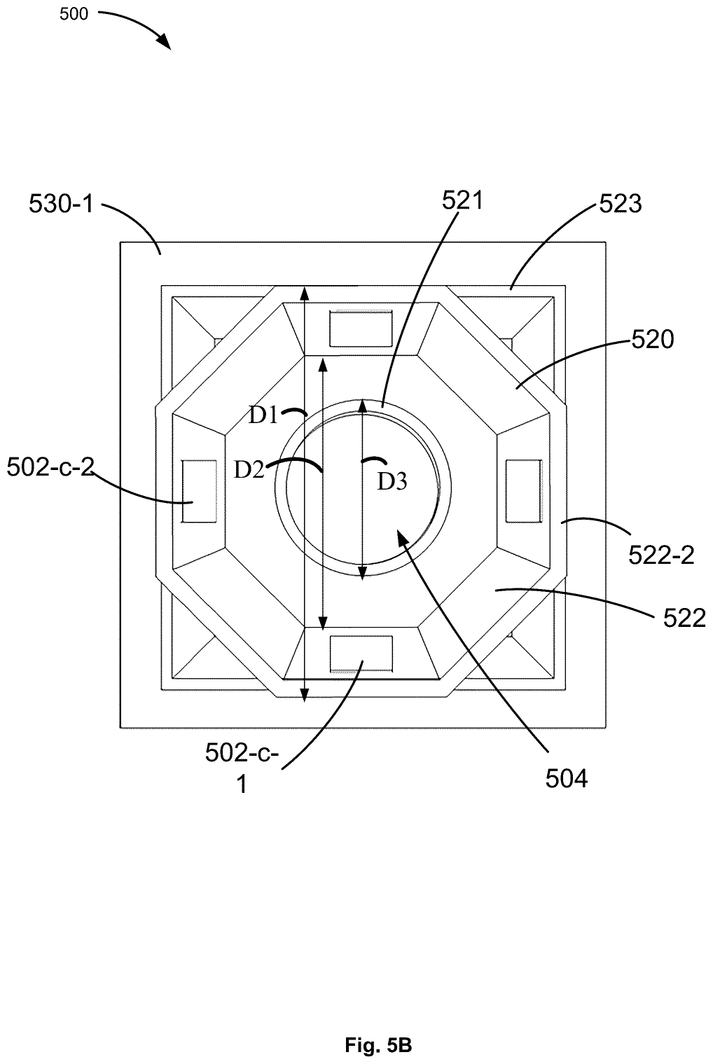

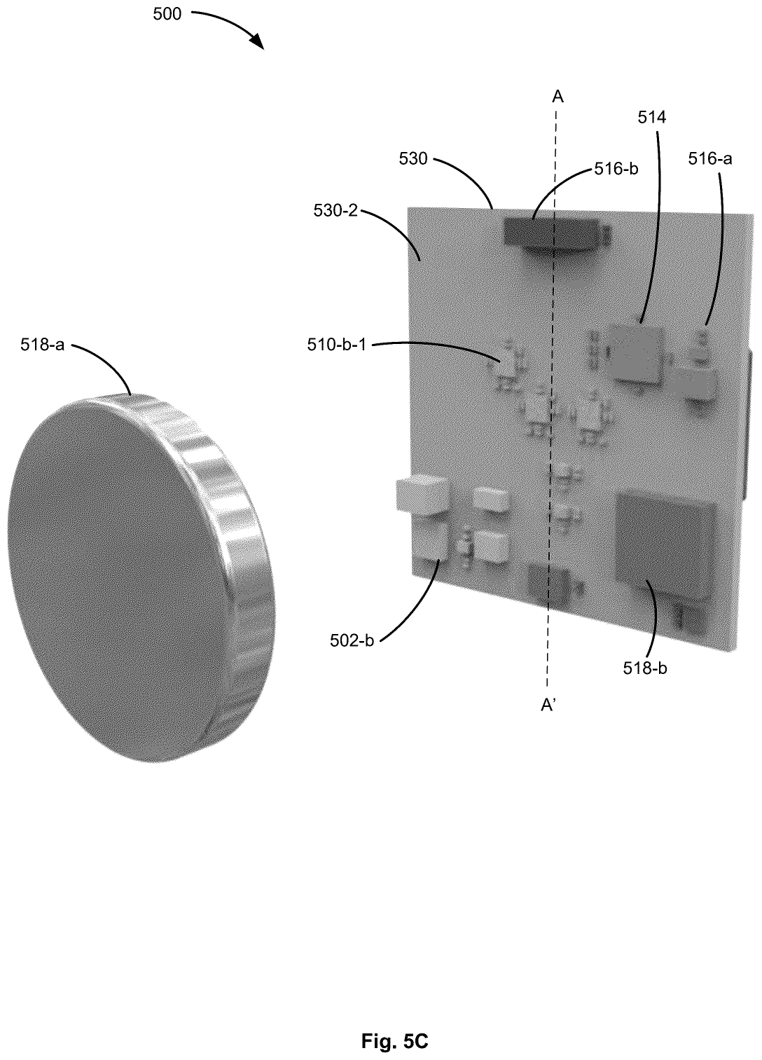

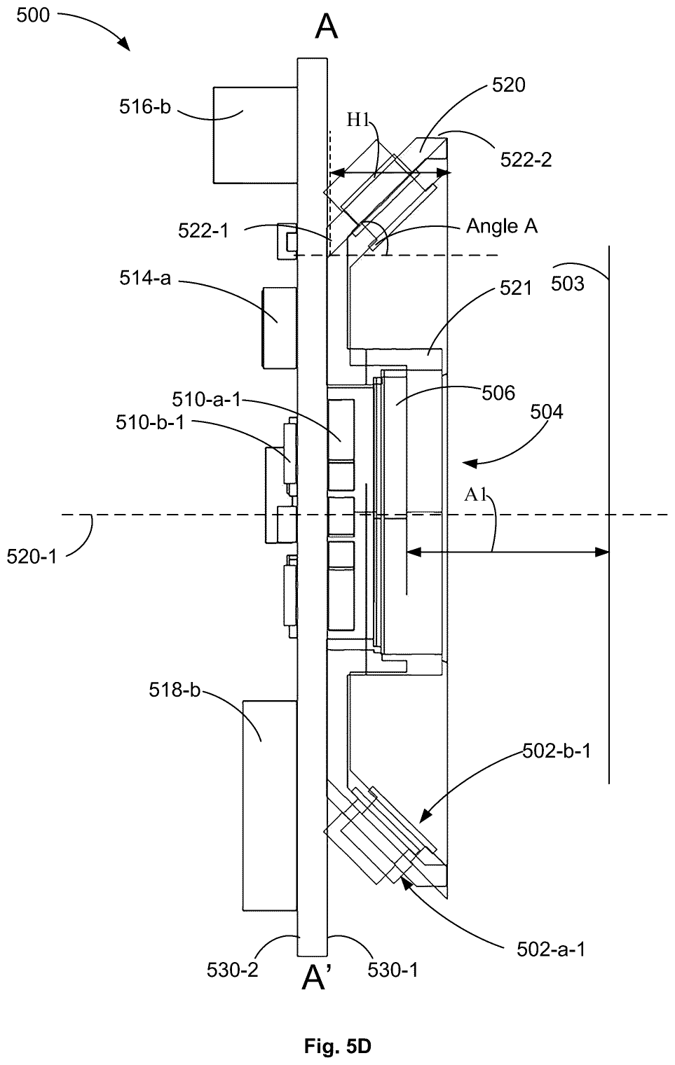

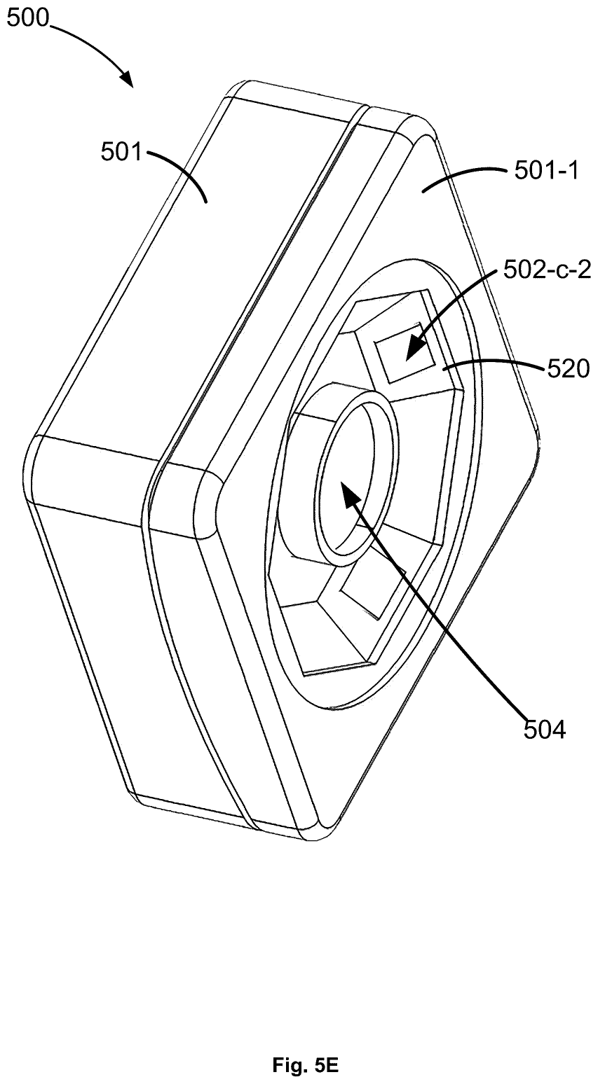

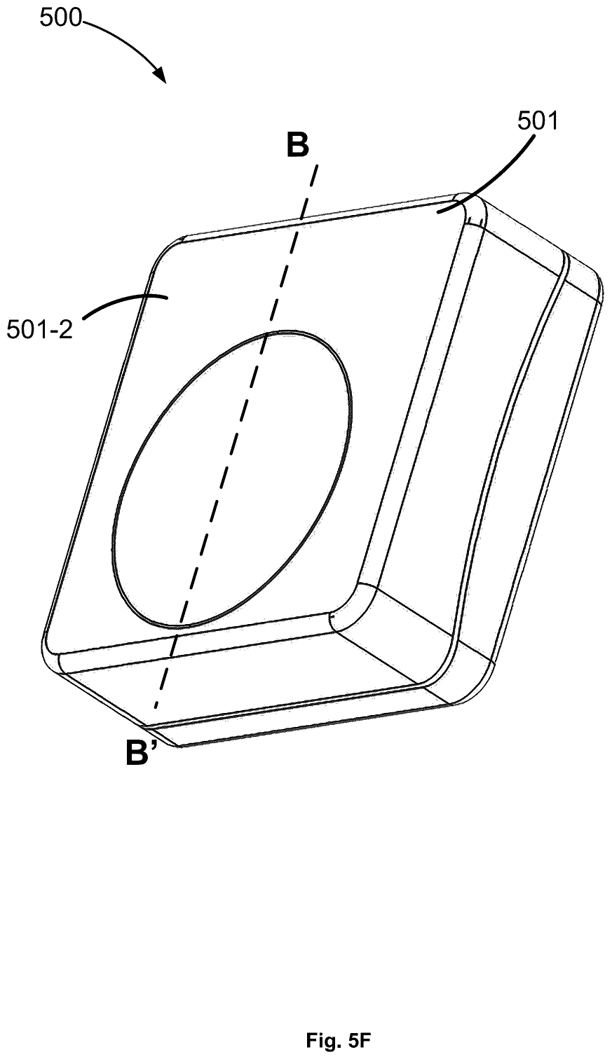

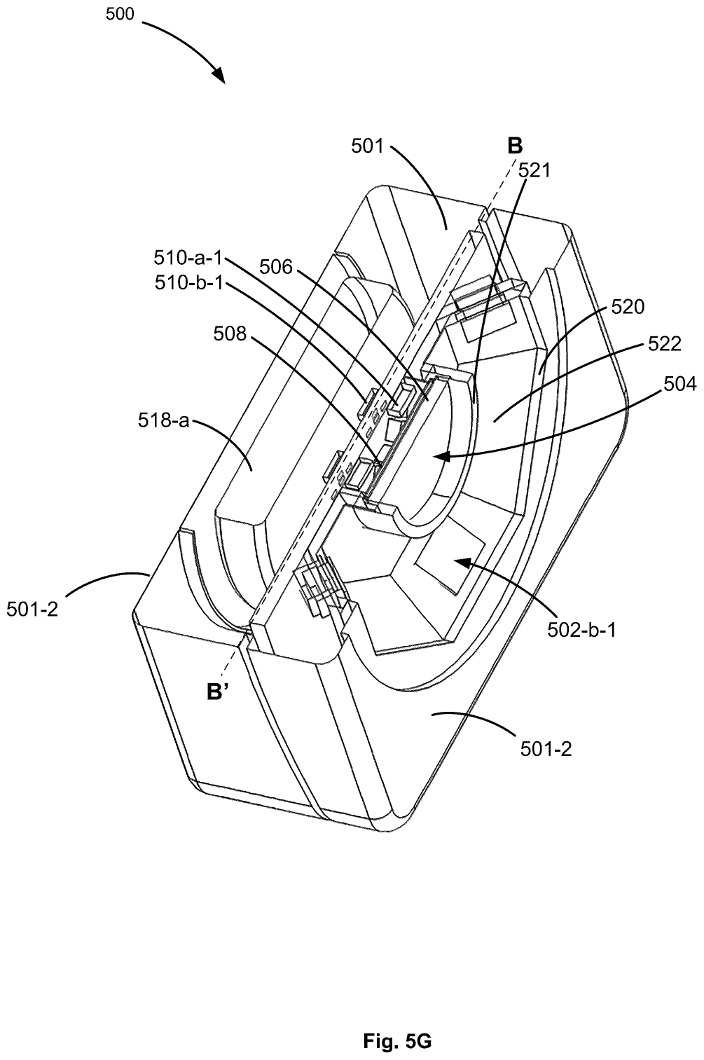

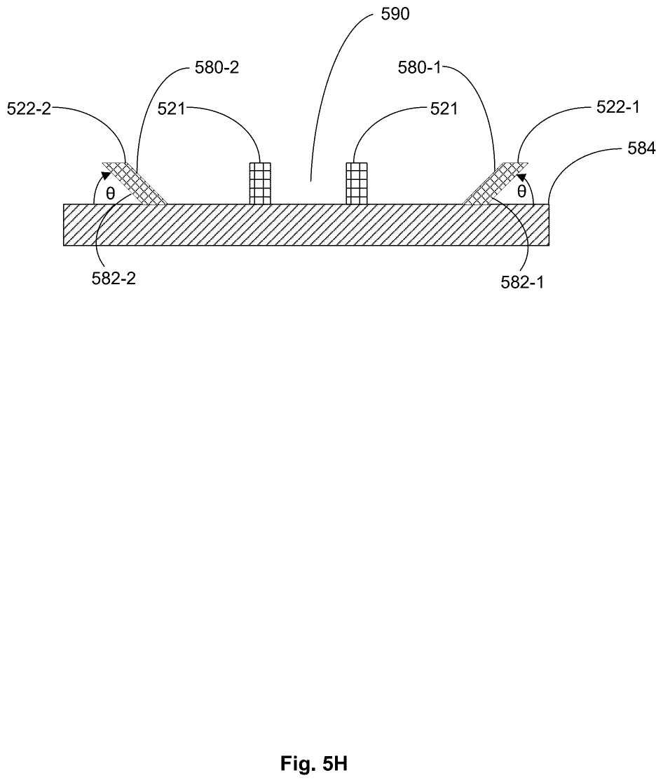

With respect to the various elements of the systems illustrated in the figures, like elements share common numerical suffixes, for ease of comparison throughout the figures. For example, elements 520, 620, and 720 all refer to a respective chassis in the various example imaging systems illustrated in FIGS. 5, 6, and 7, respectively. For brevity, an element represented in multiple figures can be referred to in the specification by using a ‘$’ followed by its common numerical suffix. For example, chasses 520, 620, and 720, as illustrated in FIGS. 5, 6, and 7, respectively, can be commonly referred to herein as a chassis $20. It will be assumed that chassis $20 refers to all of the chasses illustrated throughout the Figures. Additionally, an integer “n” where n=1, 2, 3, etc. is used to identify corresponding but distinct elements in a plurality of elements. For example, light source elements 502-a-1 and 502-a-2 are corresponding but distinct light source elements of a plurality of light source elements including elements from 502-a-1 to 502-a-n.

### DETAILED DESCRIPTION

As described above, conventional hyperspectral imaging is prohibitively expensive for use in many clinical and home healthcare settings. The lack of access to cost-effective hyperspectral imaging devices results in the use of inferior medical procedures and/or prevents regular monitoring of serious conditions that can result in limbic amputation or death. For example, diabetic foot ulcers become infected in approximately 50% of cases, which leads to a 15% rate of amputation. Reviewed in Crisologo et al., Ann Transl Med., 5 (21): 430 (2017), the content of which is incorporated herein by reference. Regular monitoring of extremities in diabetic patients greatly reduces the complication rate associated with ulceration, as problems can be identified and treated at a much earlier stage. Hyperspectral imaging is particularly well suited to assess the risk of diabetic foot ulcer development and to predict the likelihood of healing noninvasively. See, for example, Yudovsky et al., J. Diabetes Sci. Technol. 4 (5): 1099-113 (2010), the content of which is incorporated herein by reference. However, exorbitant cost has prevented the implementation of hyperspectral imaging in the home healthcare environment.

Advantageously, the present disclosure provides low-cost, compact hyperspectral imaging devices that can readily be employed by patients at home. The imaging devices described herein facilitate convenient means for patient self-monitoring at home and, in some implementations, are easily controlled by the patient's own personal electronic device (e.g., a smart phone, tablet, laptop computer, desktop computer, etc.). Further, in some implementations, the imaging devices described herein allow a physician to review the patient's condition on-demand, without requiring the patient to visit a clinical environment.

The imaging devices described herein realize these advantages because they are constructed with low-cost, fixed optical components requiring very little power for operation. Remarkably, the hyperspectral imaging device shown in FIG. 5 can then be powered by a simple watch battery.

The low power requirement of the imaging devices described herein is partially realized by enabling hyperspectral imaging at very short distances, which greatly reduces the illumination power budget for the device. Conventional hyperspectral imaging systems require high-powered illumination, in order to collect images at multiple wavebands. Lowering the illumination output of conventional hyperspectral imaging systems significantly decreases the signal-to-noise ratio, thereby decreasing image quality, detection sensitivity, and reliability. However, this problem has been solved by designing a hyperspectral imaging device operable at very short distances, e.g., as close as placing the imaging device directly onto the region of interest. To achieve this, the devices described herein include a plurality of compact-sized light sources (e.g., light-emitting diodes (LEDs)) that are positioned at an angle with respect to a region of interest (e.g., light source elements 502-a in FIGS. 5A-5G). The plurality of the light sources is further positioned around (e.g., symmetrically) an imaging subsystem (e.g., imaging unit 504) thereby ensuring a substantially uniform illumination of the region of interest. For example, the plurality of light sources are coupled with a wall of a chassis (e.g., outer wall 522 of chassis 520) that surrounds the imaging subsystem. The plurality of light sources positioned at the angle allows for irradiated light to spread across the region of interest, and provide sufficiently intense illumination, at close proximity. Positioning the plurality of light sources at the angle reduces detection of surface reflections (e.g., surface reflection corresponding to light that reflects off of a surface of a region interest without interacting with the region of interest) as the angle allows light reflect off of the surface of the region of interest to be directed away from detectors of the device.

This architecture, however, creates a new problem in that stray light from the illumination sources may reach the detectors because the illumination sources are positioned around and at an angle relative to the detectors. In some implementations, this problem is solved by including a chassis (e.g., chassis 520 in FIGS. 5A-5G) having a barrier between the plurality of light source elements and the detector (e.g., inner perimeter wall 521 of chassis 520) reducing stray light from the plurality of light source elements (e.g., light source elements 502-a) from entering the detector (e.g., imaging unit 504). For instance, as shown in Figure C, light 602-d′ emitted from light source 602-a-2 is blocked from reaching optical detector 610-a by inner perimeter wall 621 of chassis 620.

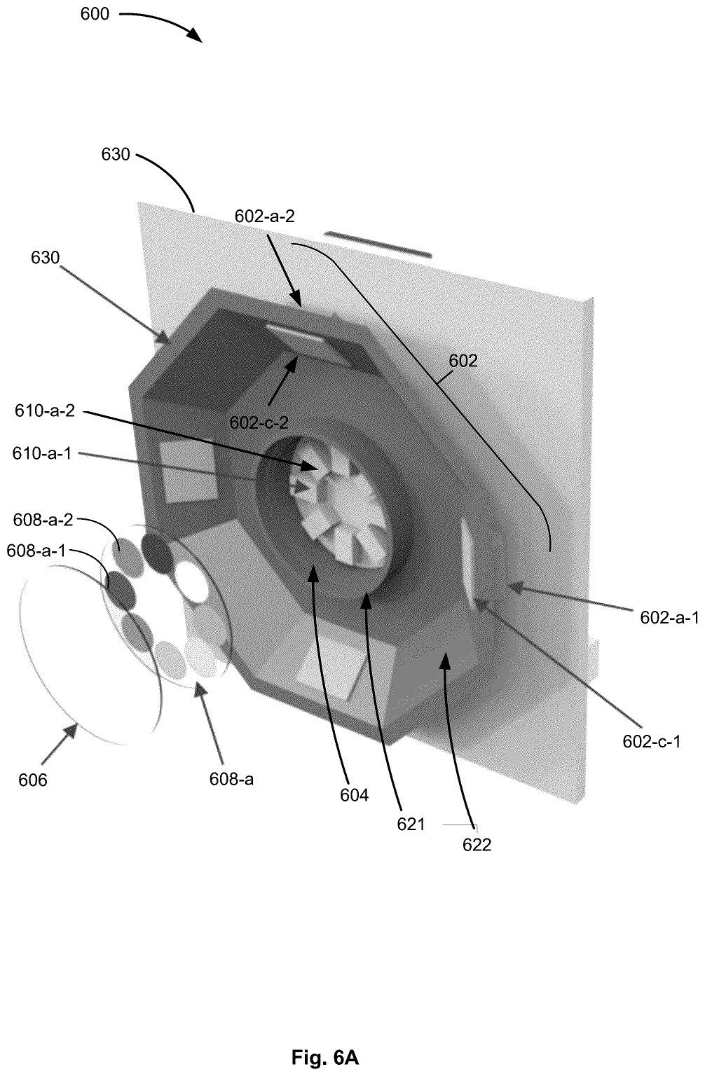

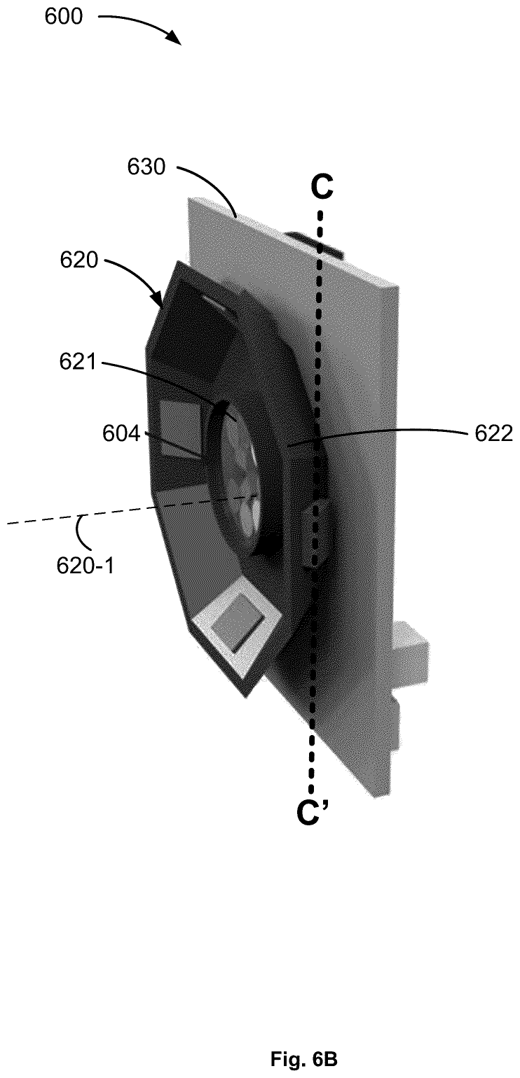

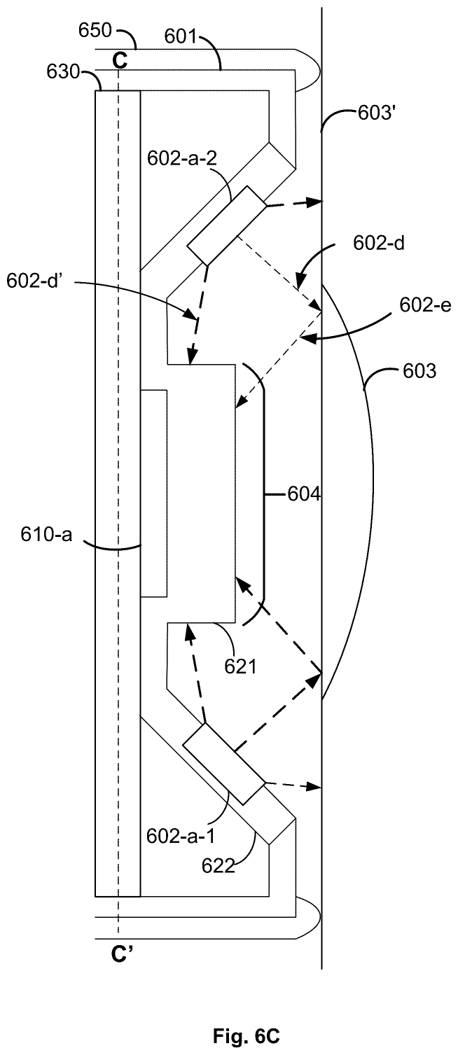

In some implementations, the illumination power requirements of the system are further reduced by including an enclosure (e.g., handheld enclosure $50), casing (e.g., casing $01), and/or opaque chassis (e.g., chassis $20) configured to prevent ambient light from entering the imaging device (e.g., imaging unit 504). For example, as illustrated in FIG. 6C, enclosure 650, which snaps around casing 601, is designed to be placed near and/or in direct contact with the skin of the subject 603′ including region of interest 603, such that ambient light is blocked from reaching detector subsystem 604, improving the signal-to-noise ratio of the imaging system.

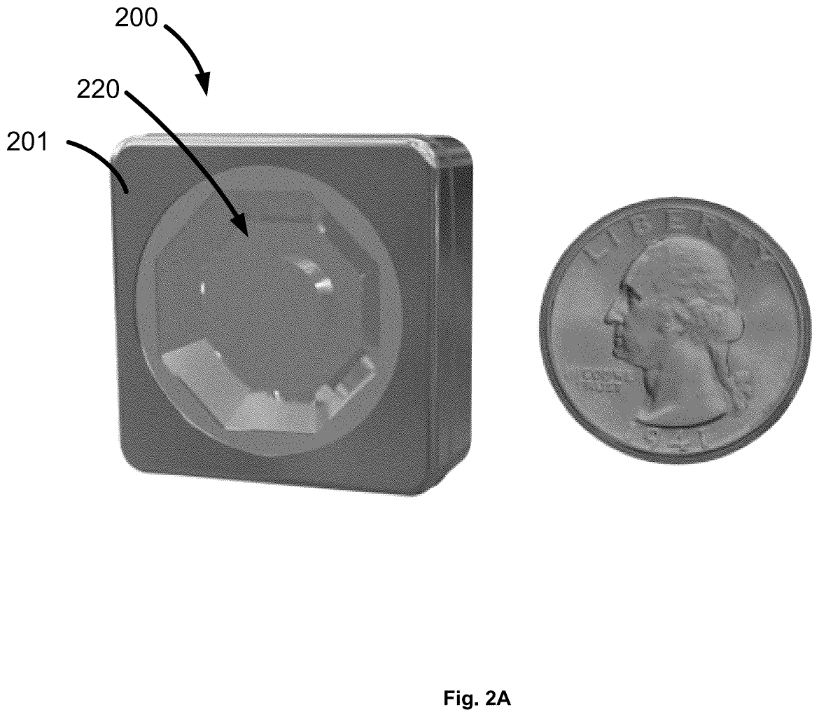

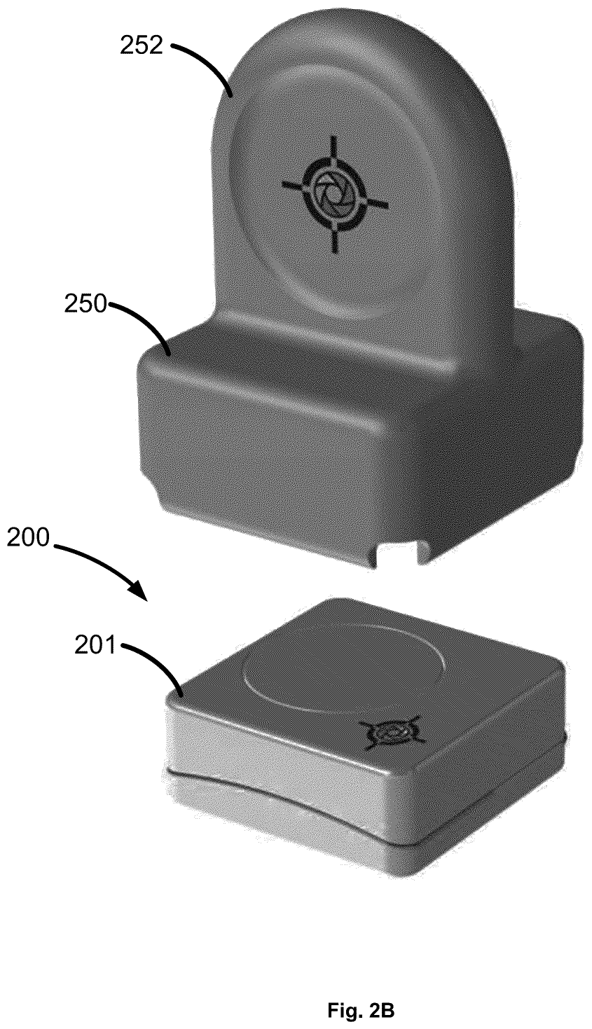

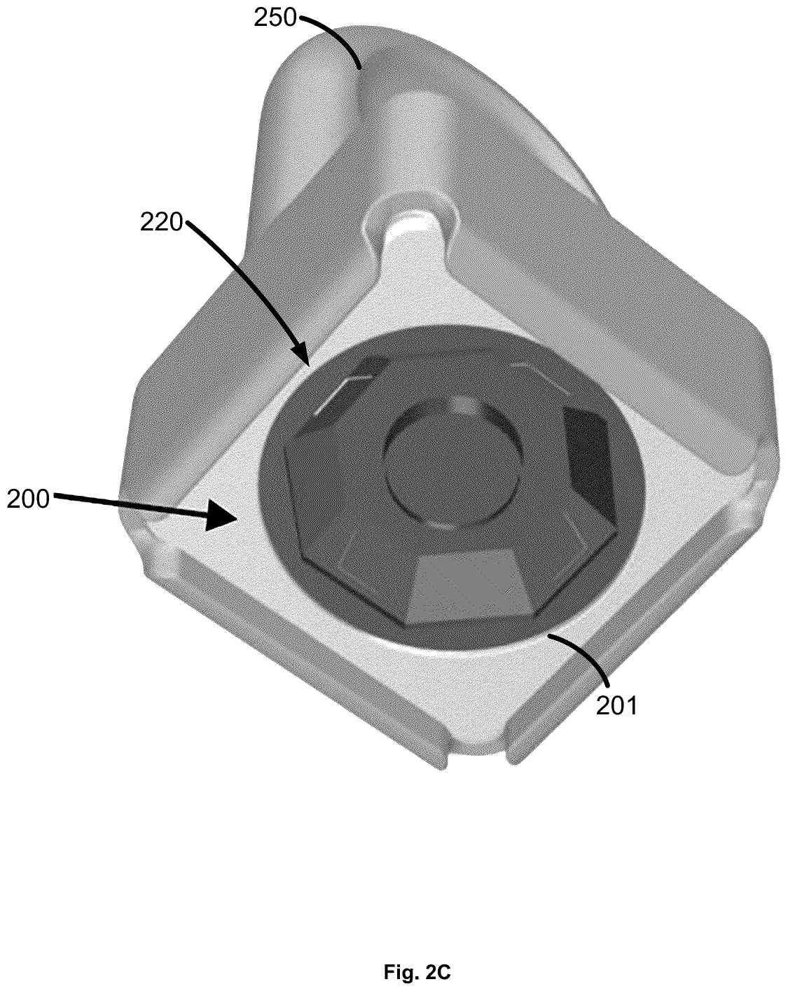

The novel design of the optical architecture of the hyperspectral imaging devices described herein enables an inexpensive solution to the problems associated with routine hyperspectral monitoring, e.g., in a home healthcare environment. As illustrated in FIGS. 2A-4, some implementations of the device are so compact that they can be incorporated as a wearable and/or hand-held detector, further improving the case and convenience of remote hyperspectral monitoring.

Reference will now be made in detail to implementations of the embodiments of the present invention as illustrated in the accompanying drawings. The same reference indicators will be used throughout the drawings and the following detailed description to refer to the same or like parts. Those of ordinary skill in the art will understand that the following detailed description is illustrative only and is not intended to be in any way limiting. Other embodiments of the present invention will readily suggest themselves to such skilled persons having benefit of this disclosure.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

Many modifications and variations of the embodiments set forth in this disclosure can be made without departing from their spirit and scope, as will be apparent to those skilled in the art. The specific embodiments described herein are offered by way of example only, and the disclosure is to be limited only by the terms of the appended claims, along with the full scope of equivalents to which such claims are entitled.

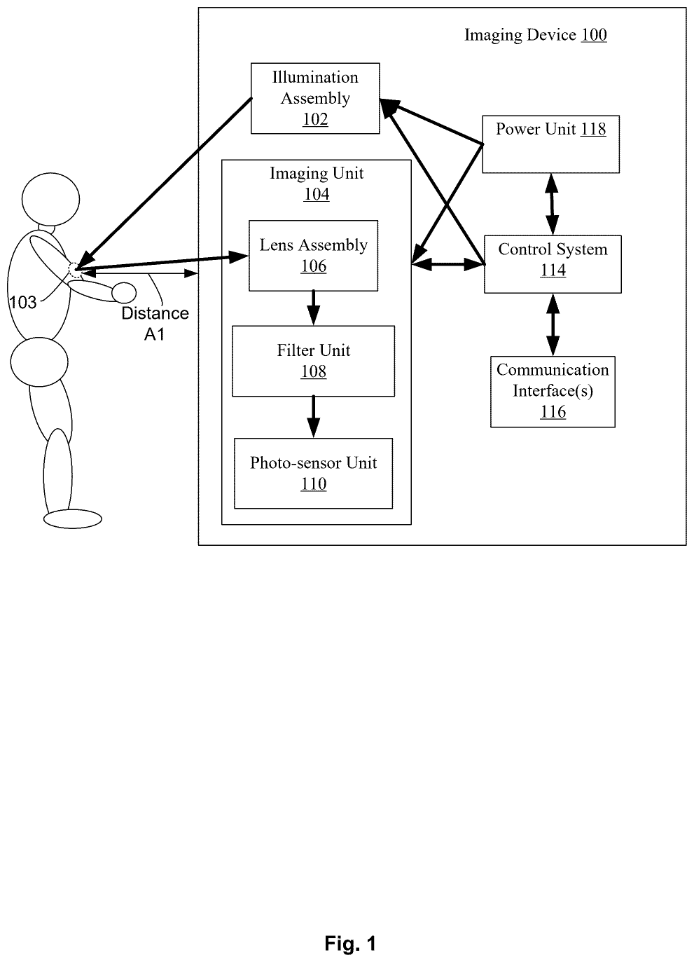

FIG. 1 is a schematic block diagram illustrating example imaging device 100 in accordance with some embodiments of the present disclosure. In some embodiments, imaging device 100 is a hyperspectral spectroscopy imaging device. Imaging device 100 includes illumination assembly 102, imaging unit 104, power unit 118, control system 114, and one or more communication interfaces 116. Illumination assembly 102 includes one or more light sources configured to illuminate a region of interest (e.g., region of interest (ROI) 103) of an object. In some embodiments, the region of interest is an area on a surface of a subject's skin. In some embodiments, ROI 103 has a size less than 700 mm², less than 650 mm², less than 600 mm², less than 550 mm², less than 500 mm², less than 450 mm², less than 400 mm², less than 350 mm², less than 300 mm², less than 250 mm², less than 200 mm², less than 150 mm², less than 100 mm², or less than 50 mm². In some embodiments, ROI 103 is located at distance A1 from imaging device 100 while capturing the hyperspectral data. In some embodiments, distance A1 has a range that is less than 50 mm, less than 40 mm, less than 30 mm, less than 25 mm, less than 20 mm, 18 mm, less than 16 mm, less than 14 mm, less than 12 mm, less than 10 mm, less than 8 mm, less than 6 mm, less than 4 mm, or less than 3 mm.

Imaging unit 104 incudes lens assembly 106, filter unit 108, and photo-sensor unit 110. Imaging unit 104 is configured to receive (e.g., collect) and detect light reflected (or backscattered) off of the illuminated ROI after interfacing with the ROI. Lens assembly 106 includes one or more lenses and/or other optical elements configured to receive light reflected off of ROI 103 and redirect (e.g., converge or focus) the light from the ROI 103 toward photo-sensor unit 110. Photo-sensor unit 110 is configured to receive the light from ROI that has been redirected by lens assembly 106 and passed through filter unit 108. Photo-sensor unit 110 includes a plurality of optical detectors (e.g., photodiodes and/or pixels of a photosensor array). Filter unit 108 includes a set of filters (e.g., a set of two or more bandpass filters). Each filter of the set of filters is configured to transmit light having a particular wavelength range while blocking (e.g., reflecting or absorbing) light having a wavelength outside the particular wavelength range. In some embodiments, each optical detector of the plurality of optical detectors in photo-sensor unit 110 is covered a respective filter of the set of filters. Such configuration allows photo-sensor unit 110 to capture a plurality of detector outputs (e.g., images) of ROI 103 simultaneously so that the detector outputs have the same illumination conditions at substantially same time. Because each optical detector of photo-sensor unit 110 receives light through a filter having a different transmittance wavelength range, each optical detector captures a different spectral component of the backscattered light. These multiple detector outputs, each representing a different spectral component, are assembled into a hyperspectral data.

Power unit 118 includes one or more power sources (e.g., one or more batteries) for running imaging device 100 so that imaging device 100 can run as a standalone device (e.g., capable of operating independently of other hardware). Power unit is in electrical communication with illumination assembly 102 (e.g., to provide power to light source devices), photo-sensor unit 110 of imaging unit 104, and/or control system 114 to provide electrical power.

Control system 114 includes one or more processors and memory. In some embodiments, the one or more processors include microprocessors (e.g., one or more central processing units (CPUs)). The one or more processors are configured to execute instructions stored in the memory that cause illumination assembly 102 and imaging unit 104 to operate. In some embodiments, the one or more programs stored in the memory include instruction for turning on light sources of illumination assembly 102 and operating photo-sensor unit 110 of imaging unit 104 to collect light, thereby generating a hyperspectral data cube of the detector outputs. In some embodiments, the one or more programs stored in the memory further include instructions for performing a spectral analysis on the plurality of detector outputs to determine a concentration value of each respective spectral signature in one or more spectral signatures associated with the ROI. However, in other embodiments, spectral analysis is performed by a second device and/or a distributed (e.g., cloud-based) computing environment. In some embodiments, the one or more spectral signatures associated with the ROI include a spectral signature of oxyhemoglobin, deoxyhemoglobin, or melanin. In some embodiments, the one or more spectral signatures are used for determination of oxygen saturation or an oximetry index value.

In some embodiments, the one or more processors are in communication with communication interface 116 for transferring data received from imaging unit 104 (e.g., data including the detector outputs and/or hyperspectral data cubes formed from the detector outputs) to, for example, a remote electronic device or a remote server. In some embodiments, communications interface 116 is in wired or wireless communication with an external device or a communication network. In some embodiments, communication interface 116 communicates the detector outputs from imaging unit 104 to the external device so that the external device performs analysis of the detector outputs (e.g., generates hyperspectral data cubes from the detector outputs).

In some embodiments, imaging device 100 further includes a display. For example, imaging device 100 includes a display for displaying measurement parameters and/or measurement results. In some embodiments, the display is coupled with imaging device. In some embodiments, the display is coupled to an internal or external surface of a casing of display device 100 (e.g., casing 201 shown in FIG. 2A). In some embodiments, the display is an external display. However, in some embodiments, device 100 does not have a display and/or measurements parameters and/or results are displayed on a second device, e.g., a personal electronic device, such as a smart phone, tablet, smart watch, laptop computer, desktop computer, etc.

FIGS. 2A, 2B, and 2C illustrate several views of example imaging module 200 in accordance with some embodiments of the present disclosure. FIG. 2A illustrates a bottom view of imaging module 200. In some embodiments, imaging module 200 includes imaging device 100 described above with respect to FIG. 1. In some embodiments, imaging module 200 is a compact-sized, light-weighted, self-powered, and self-contained hyperspectral imaging module (e.g., a standalone hyperspectral imaging module) configured for obtaining hyperspectral images of a subject's skin in a non-clinical setting. For example, imaging module 200 could be used for monitoring a patient's condition in a home setting.

As shown in FIG. 2A, imaging module 200 includes casing 201 and chassis 220. Casing 201 is configured to enclose an imaging device (e.g., imaging device 100) to provide protection and physical support. Chassis 220 provides a structural enclosure for an illumination assembly (e.g., illumination assembly 102) and an imaging unit (e.g., imaging unit 104). As shown, chassis 220 is mechanically coupled with casing 201 so that chassis 220 is surrounded by, and in direct contract with, casing 201. In FIG. 2A, imaging module 200 is illustrated, in scale, together with a quarter to demonstrate the compact size of imaging module 200.

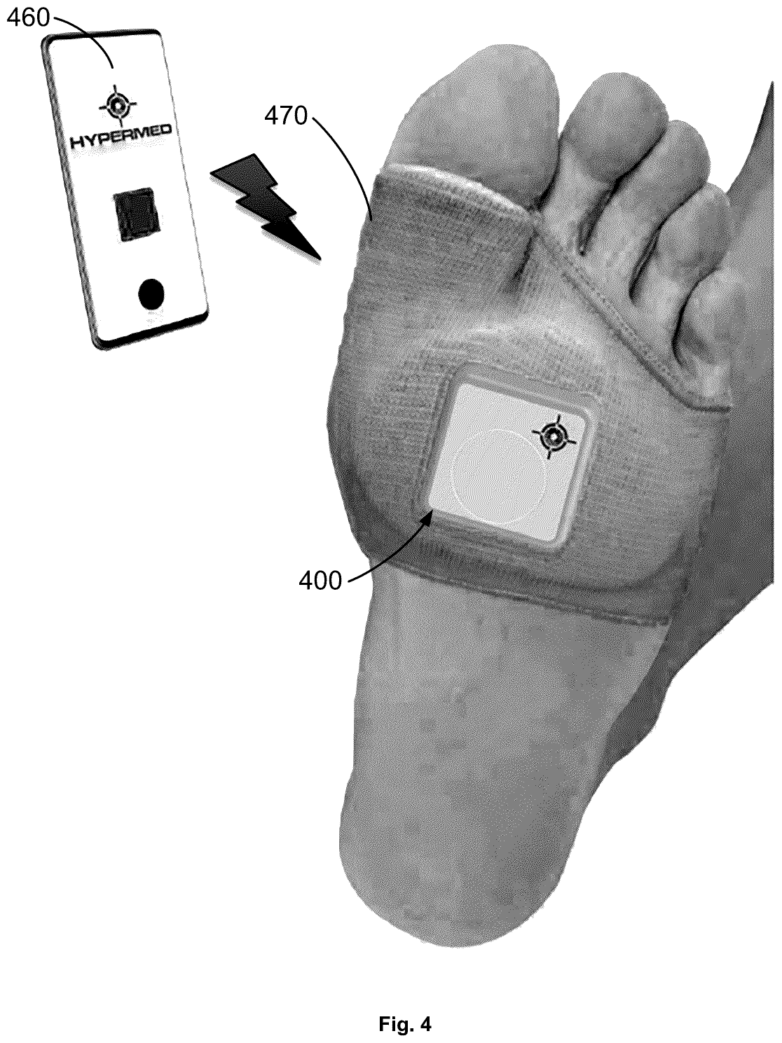

In some embodiments, imaging module 200 is attached to an enclosure or a housing. In some embodiments, imaging module 200 is configured to be attached to and detached from an enclosure. In some embodiments, the enclosure is a handheld enclosure. FIG. 2B illustrates a top-view of imaging module 200 together with an example handheld enclosure 250. A handheld enclosure allows a user to move the imaging module around such that it can be positioned and held in the vicinity of a subject's skin for collecting hyperspectral images. For example, the user holds the imaging module 200 in contact with, or in proximity to, the subject's skin for a period of time to collect the hyperspectral images. In some embodiments, the enclosure is a wearable enclosure (e.g., wearable enclosure 470 shown in FIG. 4). A wearable enclosure allows a user to position imaging module 200 in contact with, or in proximity to, the subject's skin so that the imaging module is held in position without the user actively holding it. The wearable enclosure allows, for example, for a longer time monitoring of the subject's condition and/or collection of images over a time course.

In FIG. 2B, handheld enclosure 250 is shown separately from imaging module 200 for illustrative purposes. In some embodiments, imaging module 200 is configured to be attached to and detached from handheld enclosure 250. In some embodiments, handheld enclosure 250 is at least partially surrounding casing 201 so that, when attached, imaging module 200 can be moved around by holding handheld enclosure 250. In some embodiments, casing 201 is configured to be snapped-fitted into handheld enclosure 250. In some embodiments, handheld enclosure 250 includes gripping knob 252 allowing a user to conveniently hold imaging module 200.

FIG. 2C illustrates yet another bottom-view of imaging module 200. In FIG. 2C, imaging module 200 is attached to handheld enclosure 250 so that casing 201 is partially surrounded and in direct contact with handheld enclosing 250 while chassis 220, including the illumination assembly (e.g., illumination assembly 102) and the imaging unit (e.g., imaging unit 104), is exposed (e.g., handheld enclosure 250 is not in direct contact with chassis 220 of imaging module 200). Chassis 220 is therefore open for illuminating and imaging an area of a surface of a skin of a subject (e.g., ROI 103 in FIG. 1).

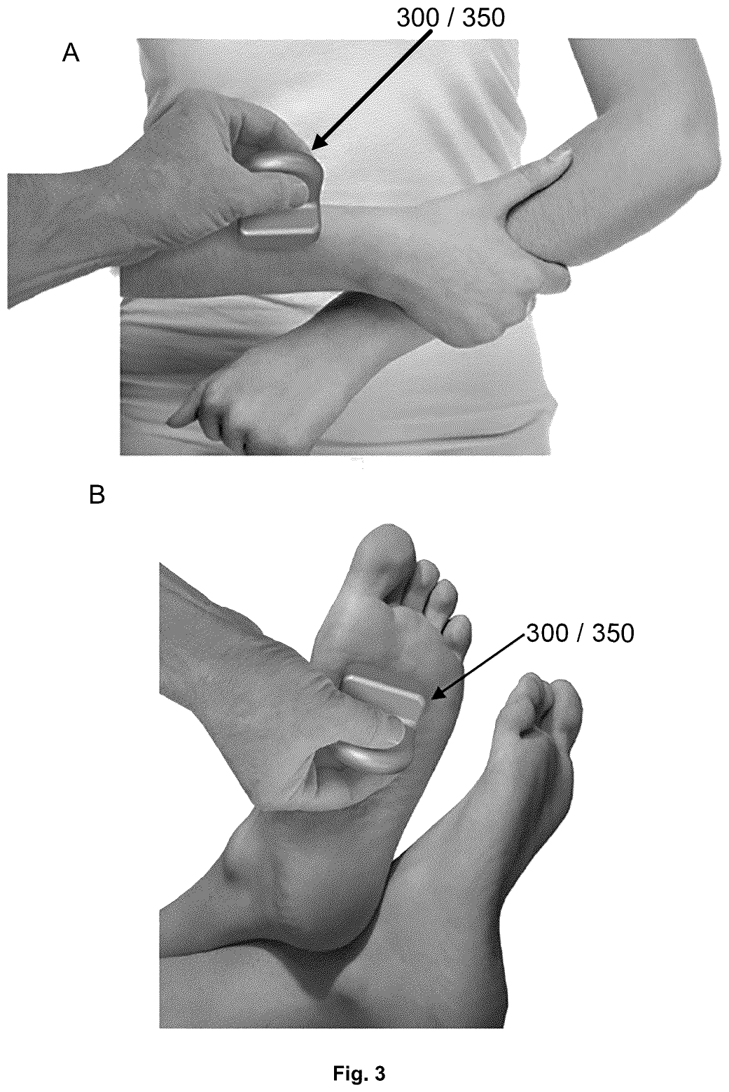

FIG. 3 illustrates the use of example imaging module 300 and handheld enclosure 350 in accordance with some embodiments of the present disclosure. In some embodiments, imaging module 300 and handheld enclosure 350, respectively, correspond to imaging module 200 and handheld enclosure 250 described with respect to FIGS. 2A-2C. In Section A of FIG. 3, a user is holding handheld enclosure 350 which is attached to imaging module 300 in contact with, or in close proximity to, a subject's arm. Imaging module 300 is used to illuminate a region of the subject's skin and collects light reflected off of the region to form hyperspectral imaging. In Section B of FIG. 3, a user is holding handheld enclosure 350 which is attached to imaging module 300 in contact with, or in close proximity to, a subject's foot (e.g., plantar area of the foot). Imaging module 300 is used to illuminate a region of the subject's skin and collects light reflected off of the region to form hyperspectral imaging.

FIG. 4 illustrates the use of imaging module 400 and wearable enclosure 470 in accordance with some embodiments of the present disclosure. In some embodiments, imaging module 400 corresponds to imaging module 200 described with respect to FIG. 2A-2C. As shown, in some embodiments, wearable enclosure 470 is configured as a partial sock to be worn on a foot of the subject. In some embodiments, wearable enclosure 470 includes a wrapper for securing imaging module 400 in contact with an ROI. In some embodiments, imaging module 400 is sleevable between the ROI and wearable enclosure 470 (e.g., the wrapper).

Alternatively, wearable enclosure 470 is configured to be worn at any body part of interest. For example, wearable enclosure 470 is a cloth worn around the subject's arm, hand, leg, head, finger, etc. In some embodiments, wearable enclosure 470 is made of a stretchable material. In some embodiments, wearable enclosure 470 is configured to be disposable while imaging module 400 can be reused by detaching imaging module 400 from wearable enclosure 470 and positioning imaging module 400 to a different wearable enclosure.

In some embodiments, imaging module 400 is in communication with a user device (e.g., user device 460). In such embodiments, imaging module 400 provides data (e.g., detector outputs and/or hyperspectral image data) to the user device. The user may then review the information on the user device. In some embodiments, the user device is a mobile device (e.g., a smartphone, a laptop, or a tablet computer) or a desktop device (e.g., a personal computer). In FIG. 4, imaging module 400 is in communication with user device 460 thereby allowing the user to conveniently and simultaneously monitor the subject's condition by reviewing the information from user device 460. In some embodiments, device 460 further communicates with an external server and/or a distributed cloud computing environment, which analyzes the hyperspectral data collected by imaging module 400 and/or communicates medical information obtained from the hyperspectral data to a medical professional. In some embodiments, imaging module 400 is directly in communication with the external server and/or the distributed cloud computing environment. Examples of remote server-based hyperspectral analysis are described in U.S. Patent Application Publication No. 2015/0142461, the content of which is expressly incorporated by reference herein, in its entirety, for all purposes. Similarly user device 460 could be used in communication with imaging module 300 described with respect to FIG. 3.

FIGS. 5A, 5B, 5C, 5D, 5E, 5F, and 5G illustrate several views of an example imaging device 500 in accordance with some embodiments of the present disclosure. In some embodiments, imaging device 500 corresponds to imaging device 100 described above with respect to FIG. 1. In some embodiments, imaging device 500 is a hyperspectral imaging device suitable for use outside a clinical setting. For example, imaging device 500 can be used for monitoring a patient's condition at home. In some embodiments, imaging device 500 is a standalone device capable of operating independently of other hardware. In some embodiments, imaging device 500 is configured as self-powered, self-contained, and wireless. FIGS. 5A, 5B, 5C, and 5D illustrate several views of the internal architecture of imaging device 500. In some embodiments, imaging device 500 includes board 530 configured for providing physical support for different optical and electronic components of imaging device 500. In some embodiments, board 530 is also configured to act as a divider between the optical and electronic components such that a first side of board 530 (e.g., side 530-1 shown in FIG. 5A) is coupled with the optical components and an opposing second side (e.g., side 530-2 shown in FIG. 5D) is coupled with the electronic components. In some embodiments, first side 530-1 faces an exterior of imaging device 500, and second side 530-2 faces the interior of imaging device 500. In some embodiments, the optical components include illumination assembly 502 and imaging unit 504. In some embodiments, illumination assembly 502 and imaging unit 504 correspond to illumination assembly 102 and imaging unit 104, respectively, described with respect to FIG. 1.

Illumination assembly 502 and imaging unit 504 are configured around chassis 520 that is mechanically coupled with board 530. Chassis includes an inner perimeter wall (e.g., inner perimeter wall 521) and one or more outer walls (e.g., outer wall 522) surrounding the inner perimeter wall. In some embodiments, the single outer wall 522 illustrated in FIG. 5 is broken up into multiple outer walls. In such embodiments where there are multiple outer walls, the multiple outer walls face the inner perimeter wall. In some embodiments, chassis 520 is symmetric with respect to a geometric center axis (e.g., axis 520-1 shown in FIG. 5A). In some embodiments, geometric center axis 520-1 of chassis 520 is substantially normal to a surface (base face) of board 530. In some embodiments, geometric center axis 520-1 of chassis 520 substantially corresponds to an optical axis of imaging device 500, such that geometric axis 520-1 of chassis 520 substantially corresponds to an optical axis of imaging unit 504 and illumination assembly 502.

As shown, the outer wall 522 (which can be one or more outer walls) is coupled with side 530-1 of board 530 such that a first surface (e.g., surface 522-1 shown in FIG. 5D) of outer wall 522 is in direct contact with board 530 and a second opposing surface (e.g., surface 522-2) is extending away from side 530-1.

Each respective outer wall 522 in the one or more respective outer walls has a respective inward face 580 facing the inner perimeter wall 521 and a respective outward face 582 opposing the respective inward face. The respective outward face 582 of each of the one or more outer walls 522 is extended at a corresponding acute angle (θ) with respect to the base face 584 as illustrated in FIGS. 5A and 5H. In particular, FIG. 5H is a side view of the perspective view of FIG. 5A, showing the inner perimeter wall 521, respective walls 522-1 and 522-2 with their inward and outward faces 580/582, the base face 584 of the chassis and the acute angle between the base face 584 and the respective outward face 582 of each of the outer walls 522. FIG. 5H further illustrates the interior region 590 of the chassis formed by the inner perimeter wall.

Outer wall 522 extends at an acute angle (e.g., Angle A shown in a cross-sectional view of imaging device 500 in FIG. 5D) with respect to the geometric axis of chassis 520 from side 530-2 of board 530. In some embodiments, the acute angle at which the outer wall 522 is extended with respect to geometric axis 520-1 of chassis 520 is between 15 degrees and 30 degrees, between 30 degrees and 45 degrees, between 45 degrees and 60 degrees, or between 60 degrees and 75 degrees.

In some embodiments, outer wall 522 defines a truncated conical structure (e.g., a conical structure with a flat apex). In some embodiments, the truncated conical structure has a circular, oval, or polygonal cross-section. In some embodiments, the polygonal cross-section is rectangular (e.g., a square), hexagonal, octagonal, or decagonal. In FIG. 5B, surface 522-2 of outer wall 522 has an octagonal cross-section. FIG. 5B also illustrates an alternative outer wall 523 having a square shape. For example, peripheral surface 522-1 defines a smaller nominal diameter than peripheral surface 532-2. In such embodiments, a nominal diameter of the truncated conical structure at the second side (e.g., D1 shown in FIG. 5B) of chassis 520 is between 10 mm and 15 mm, between 15 mm and 20 mm, between 20 mm and 25 mm, or between 25 mm and 30 mm and a nominal diameter of the truncated conical structure at the first side (e.g., D2 shown in FIG. 5B) of chassis 520 is between 15 mm and 25 mm, between 25 mm and 35 mm, or between 35 and 45 mm. In some embodiments, the truncated conical structures have a height (e.g., height H1 illustrated in FIG. 5D) along geometric axis 520-1 of chassis 520 that is less than 14 mm, less than 12 mm, less than 10 mm, less than 8 mm, or less than 6 mm.

In some embodiments, illumination assembly 502 includes one or more light sources 502-a-n (e.g., light sources 502-a-1 and 502-a-2). In some embodiments, illumination assembly includes three, four five, six, seven, eight, or more than eight light sources. In some embodiments, light sources 502-a-1 and 502-a-2 are coupled with outer wall 522 of chassis 520. As shown, each outer wall 522 is angled away from inner perimeter wall 521, such that light output from light sources 502-a-1 and 502-a-2 of illumination assembly 502 propagates to exit imaging device 500 to illuminate a region of interest (e.g., ROI 103 in FIG. 1).

In some embodiments, light sources 502-a of illumination assembly 502 are distributed over outer chassis 522 uniformly. In some embodiments, light sources 502-a are distributed symmetrically around a geometrical center axis (e.g., axis 520-a) of chassis 520. As shown in FIG. 5A, outer wall 522 of chassis 520 has an octagonal shape so that outer wall 522 includes eight distinct facets. Four light sources 502-a-n are positioned on every second facet so that the light sources are uniformly distributed around inner perimeter wall 521 including imaging unit 504. In some embodiments, eight light sources 502-a-n are positioned on each of the facets of the octagonal outer wall 522. However, these example light distributions do not limit the orientations that can be implemented in the devices described herein. For instance, in some embodiments, each facet includes more than one light source. In some embodiments, matching light sources—that is, light sources producing the same wavebands of light—are positioned on opposite facets, e.g., light sources 502-a-1 and 502-a-2 are configured to emit light of the same waveband, while light sources positioned on different opposing facets of the chassis are configured to emit light of a different waveband.

In some embodiments, light sources 502-a-1 and 502-a-2 are incandescent lights, xenon lamps, halogen lamps, hydrargyrum medium-arc iodide, and broadband light emitting diodes (LEDs), or any combination thereof. In some embodiments, light source elements 502-a-n are configured to emit near infrared (NIR) light, visible light, ultraviolet light, or any combination thereof. In some embodiments, light sources 502-a-1 and 502-a-2 output light within a common wavelength range (e.g., a broad wavelength range). In some embodiments, each light source 502-a is configured to emit white light having a spectrum between 400 nm and 780 nm. In some embodiments, light sources 502-a-1 and 502-a-2 (e.g., laser diodes or narrow bandwidth light emitting diodes) output light with distinct wavelength ranges. For example, light source 502-a-1 provides light having a first wavelength range, and light source element 502-a-2 provides light having a second wavelength range distinct from the first wavelength range. In such embodiments, light sources 502-a-1 and 502-a-2 output light sequentially so that light source 502-a-1 provides the light having the first wavelength range at a first time and light source 502-a-2 provides the light having the second wavelength range at a second time that is distinct and not concurrent with the first time. Consequently, imaging unit 504 is configured to capture images sequentially. For example, imaging unit 504 captures a first image of a region of interest illuminated with the light having the first wavelength range at the first time and a second image of the region of interest illuminated with the light having the second wavelength range at the second time.

In some embodiments, illumination assembly 502 includes one or more optical filters 502-c (e.g., optical filter 502-c including filter elements 502-c-1 502-c-2). The one or more optical filters 502-c are in optical communication with one or more light source elements 502-a-n. The one or more optical filters 502-c are configured to receive light from one or more light source elements 502-a-n and transmit a portion of the light toward an ROI (e.g., ROI 103 in FIG. 1). In some embodiments, illumination assembly 502 includes three, four five, six, seven, eight, or more than eight light source filters 502-c. In some embodiments, each light source element 502-a-n is optically coupled with a respective filter 502-c. As shown, filter 502-c-1 optically coupled with light source element 502-a-1. In some embodiments, filters 502-c are bandpass filters configured to transmit light having a particular wavelength range while blocking (e.g., reflecting or absorbing) light having a wavelength outside the particular wavelength range (e.g., the optical filters 502-c have a spectral band corresponding to the particular wavelength range). As such, each filter 502 has a particular band-pass range, the range of light the filter permits to pass. In some embodiments, this band-pass range is one contiguous range of wavelengths. In some embodiments, this band-pass range it two different non-overlapping contiguous ranges of wavelengths. In some embodiments, optical filters 502-c are configured to transmit light with a distinct wavelength range. For example, in some embodiments, optical filter 502-c-1 is configured to transmit light having a first wavelength range and optical filter 502-c-2 is configured to transmit light having a second wavelength range distinct from the first wavelength range. In some embodiments, at least two optical filters of optical filters 502-c are configured to transmit light having a common wavelength range. In some embodiments, optical filter 502-c-1 positioned opposite to each other on outer wall 522 of chassis, such as optical filters 502-c-1 and 502-c-2, are configured to transmit light having a common wavelength range. By illuminating an ROI with the common wavelength range symmetrically from light source elements positioned at opposing sides and at an equal distance of the geometric center of chassis 520 enables uniform illumination of the ROI with that wavelength range. In some embodiments, at least two optical filters of optical filters 502-c are configured to transmit light having a first wavelength range, and at least two optical filters of optical filters 502-c are configured to transmit light having a second wavelength range distinct from the first wavelength range. In such embodiments, the optical filters transmitting the first wavelength range are positioned symmetrically with respect to the geometric center of chassis 520 and the optical filters transmitting the second wavelength range are also positioned symmetrically with respect to the geometric center (e.g., axis 520-1) of chassis 520. Such configuration provides uniform illumination of an ROI with lights having the first and the second wavelength range, respectively.

As explained above, in some embodiments, light source elements 502-a output light with distinct wavelength ranges sequentially. In such embodiments, optical filters 502-c may be omitted.

In some embodiments, illumination assembly 502 includes one or more polarizers. For example, filter elements 502-c-1 502-c-2 include polarizers. A polarizer allows light having a particular polarization (e.g., p-polarized or s-polarized light) to pass through while reflecting or absorbing light having the opposite polarization. The use of polarized illumination is advantageous because it eliminates surface reflection from the skin and helps to eliminate stray light reflection from off-axis imaging directions. Accordingly, in some implementations, polarized light is used to illuminate the object being imaged. In some implementations, the light is polarized with respect to a coordinate system relating to the plane of incidence formed by the propagation direction of the light and a vector perpendicular to the plane of the reflecting surface (e.g., the object being imaged). The component of the electric field parallel to the plane of incidence is referred to as the p-component and the component perpendicular to the plane is referred to as the s-component. Accordingly, polarized light having an electric field along the plane of incidence is “p-polarized,” while polarized light having an electric field normal to the plane is “s-polarized.” Advantageously, in some embodiments, imaging device 500 recaptures and reverses the polarity light reflected off the polarization filter, using a polarization rotator (e.g., a polarization rotation mirror).

In some embodiments, imaging device 500 is configured to be positioned in contact with a surface of an object (e.g., an area of skin of a patient as shown in FIGS. 3 and 4) such that surface 522-2 of outer wall 522, facet 501-1 of a casing, and/or the surface of a housing 250 (e.g., as illustrated in FIG. 2C), is in direct contact with the surface of the object. In some embodiments, outer wall 522, and/or housing 250, is made of an oblique material. The oblique material is configured to prevent outside light (e.g., ambient light) from entering imaging unit 504 when imaging device 500 is positioned in contact with skin thereby reducing background noise and improving detection sensitivity and image quality of the hyperspectral images.

Inner perimeter wall 521 is coupled with imaging unit 504 such that imaging unit 504 is surrounded by inner perimeter wall 521 from all edges of imaging unit 504. In some embodiments, inner perimeter wall 521 extends substantially along the geometric axis of chassis 520 from side 530-2 to side 530-1 of board 530. In some embodiments, chassis 520 is partially embedded inside board 530. In some embodiments, inner perimeter wall 521 has a cylindrical shape. In some embodiments, inner perimeter wall 521 defines a nominal diameter (e.g., D3 shown in FIG. 5B) between 5 mm and 10 mm, between 10 and 15 mm, or between 15 and 20 mm. In some embodiments, inner perimeter wall 521 has a height defined along the geometric axis of chassis 520 that is less than 10 mm, less than 8 mm, less than 6 mm, or less than 4 mm. In some embodiments, inner perimeter wall 521 is a cylindrical structure having a circular, oval, or polygonal (e.g., a square-shaped) cross-section.

FIG. 5B illustrates a view of first side 530-1 of board 530 of imaging device 500 in accordance with some embodiments. First side 530 is coupled with chassis 520. Chassis 520 includes inner perimeter wall 521 and outer wall 522. As described above, outer wall 522 is coupled with components of illumination assembly (e.g., optical filters 502-c-1 and 502-c-2) and inner perimeter wall 521 is coupled with imaging unit 504 (e.g., inner perimeter wall 521 is positioned surrounding imaging unit 504). The view illustrated in FIG. 5 corresponds to a side of imaging device 500 that is facing the subject's skin during collection of hyperspectral images. In some embodiments, as described above, surface 522-2 of outer wall 522, facet 501-1 of a casing, and/or the surface of a housing 250, is configured to be positioned in direct contact with the subject's skin as illustrated in FIGS. 3 and 4.

FIG. 5B illustrates dimension D1 corresponding to the nominal diameter of the truncated conical structure of outer wall 522 at surface 522-2 and dimension D2 corresponding to the nominal diameter of the outer wall 522 at a surface (e.g., surface 522-1 in FIG. 5D) of outer wall 522. FIG. 5B also illustrates dimension D3 corresponding to the nominal diameter of inner perimeter wall 521. The values associated with diameters D1, D2, D3 are described with respect to FIG. 5A and without a casing. In some embodiments, dimension D1 ranges from 10 mm to 40 mm, dimension D2 ranges from 10 mm to 25 mm, and dimension D3 ranges from 5 mm to 15 mm. In an exemplary embodiment, dimension D1 is 25 mm, dimension D2 is 16.8 mm, and dimension D3 is 9.8 mm.

FIG. 5C illustrates a view of second side 530-2 of board 530 of imaging device 500 in accordance with some embodiments. Second side 530 is coupled with electronic components of imaging device 500, including power unit 118, one or more processors of control system 114, and communication interface 116.

As explained above, imaging device 500 is configured to collect hyperspectral imaging at a close distance. Illuminating an object at such close distance requires a power that can be provided by a simple, low voltage battery. In some embodiments, imaging device 500 includes one or more power sources (e.g., battery 518-a) that are part of power unit 518 in FIG. 1. In some embodiments, the one or more power sources include one or more of lithium button cell and/or lithium polymer batteries. In FIG. 5C, battery 518-a is shown separately from imaging device 500 for illustrative purposes. Battery 518-a is in reality coupled with imaging device 500 (e.g., battery 518-a is positioned inside casing 201 described above with respect to FIGS. 2A and 2B). Battery 518-a is in electrical communication with, and is configured to provide electrical power to, imaging unit 504, illumination assembly 102 (e.g., light source elements 502-a), and one or more processors (e.g., CPUs 514-a) and is configured to provide electrical power for operating them. In some embodiments, battery 518-a has a nominal voltage of 10 volts or less. In some embodiments, battery 518-a has a nominal voltage of 10 volts or less, 9 volts or less, 8 volts or less, 7 volts or less, 6 volts or less, 5 volts or less, 4 volts or less, or 3 volts or less. In some embodiments, imaging device 500 includes one or more power regulators 518-b (e. g., one or more voltage regulators) that are part of power unit 118 described with respect to FIG. 1. One or more power regulators 518-b are in electronic communication with battery 518-a and control unit 114. One or more power regulators 518-b are configured to maintain a power supply of imaging device 500 at a desired level.

In some embodiments, imaging device 500 includes one or more processors (e.g. microprocessors such as CPUs 514-a). In some embodiments, one or more processors 514-a are part of control unit 114. One or more processors 514-a execute instructions for operating imaging device 500. In some embodiments, imaging device 500 further includes a communication interface (e.g., a communication interface corresponding to communication interface 116 described above with respect to FIG. 1). In some embodiments, communication interface includes wireless controller 516-a and antenna 516-b. Wireless controller 516-a and antenna 516-b are configured to enable communication between imaging device 500 and a remote electronic device or a server. For example, wireless controller 516-a and antenna 516-b enable communication between imaging device and a user device (e.g., user device 460 described above with respect to FIG. 4). In some embodiments, communications interface is a BLUETOOTH® interface (e.g., wireless controller 516-1 is a BLUETOOTH® controller and antenna 516-b is a BLUETOOTH® antenna). In alternatively embodiments, communications interface is a WI-FI® interface. In some embodiments, wireless controller 516-a is integrated into control unit 114.

In some embodiments, imaging device 500 further includes sensor controller 510-b-1 (e.g., a sensor conditioner) and light source drive 502-b, as shown in FIG. 5C. Sensor controller 510-b-1 is part of imaging unit 504 and light source drive 502-b is part of illumination assembly 502. Sensor controller 510-b-1 and light source drive 502-b are in electrical communication with one or more processors 514-a, battery 518-a, and/or power regulator 518-b that enable their operations. Sensor controller 510-b-1 is configured to operate one or more optical detectors of imaging unit 504. Light source drive 502-b is configured to operate light source elements 502-a (e.g., light source elements 502-a-1 and 502-a-2 shown in FIG. 5A). For example, light source drive 502-a controls the duration of illumination by turning light source elements 502-a on and off. Furthermore, light source drive 502-a controls the power of illumination by controlling an intensity of emission output by light source elements 502-a. In some embodiments, light source elements 502-a are LEDs and light source drive 502-b is a LED drive.

FIG. 5D illustrates a cross-sectional view of imaging device 500 in accordance with some embodiments. The cross-sectional view is presented along a reference line AA′ shown in FIG. 5C. Side 530-1 of board 530 is coupled with optical components of imaging device 500 and side 530-2 of board 530 is coupled with electrical components of imaging device 500. As shown, side 530-2 is coupled with wireless antenna 516-b, one or more processors 514-a, and power regulator 518-b. In some embodiments, wireless antenna 516-b is integrated into one or more processors 514-a (e.g., wireless antenna 516-b is formed on one or more printed circuit boards of one or more processors 514-a). Side 530-2 is further coupled with sensor controllers 510-b-1 coupled with optical detector element 510-a-1 of imaging unit 504 that are positioned on side 530-1. FIG. 5D also illustrates chassis 520 including inner perimeter wall 521 and outer wall 522. As explained above with respect to FIG. 5A, outer wall 522 is extended in an acute angle (e.g., Angle A) with respect to axis 520-1 from board 530. Outer wall 522, therefore, has a truncated conical shape. In some embodiments, the truncated conical structures have height H1 along geometric axis 520-1 of chassis 420 that is less than 14 mm, less than 12 mm, less than 10 mm, less than 8 mm, or less than 6 mm. As shown, height H1 is defined as a distance along geometric axis 520-1 between surface 522-1 and surface 522-2 of outer wall 522 of chassis 520. As shown, light source element 502-a-1 and optical filter 502-c-1 are coupled with outer wall 522.

As explained above, light sources 502-a are configured to illuminate a region of interest (e.g., region of interest (ROI) 503) of an object. In some embodiments, the region of interest is an area on a surface of a subject's skin. In some embodiments, ROI 503 has a size less than 700 mm², less than 650 mm², less than 600 mm², less than 550 mm², less than 500 mm², less than 450 mm², less than 400 mm², less than 350 mm², less than 300 mm², less than 250 mm², less than 200 mm², less than 150 mm², less than 100 mm², or less than 50 mm².

In some embodiments, ROI 503 is located at distance A1 from imaging device 500 while capturing the hyperspectral data. As shown in FIG. 5D, distance A1 is defined as a distance between ROI 503 and lens assembly 506 of imaging device 500. In some embodiments, distance A has a range that is less than 50 mm, less than 40 mm, less than 30 mm, less than 25 mm, less than 20 mm, 18 mm, less than 16 mm, less than 14 mm, less than 12 mm, less than 10 mm, or less than 8 mm.

FIGS. 5E and 5F illustrate several views of the exterior of imaging device 500 in accordance with some embodiments. FIG. 5E illustrates imaging device 500 enclosed with casing 501. In some embodiments, casing 501 corresponds to casing 201 described above with respect to FIG. 2A. Casing 501 includes facet 501-1 positioned adjacent to the portion of imaging device 500 including illumination assembly 502 and imaging unit 504. FIG. 5E also illustrates chassis 520 coupled with imaging unit 504 and optical filter 502-c-1. FIG. 5F illustrates imaging device 500 with facet 501-2 that is opposite to facet 501-1. Facet 501-2 corresponds to the portion of imaging device 500 including electrical components (e.g., those described above with respect to FIG. 5C).

FIG. 5G illustrates a cross-sectional view of imaging device 500, in accordance with some embodiments. The cross-sectional view of FIG. 5G illustrates a cross-section of imaging device 500 along reference line BB′ shown in FIG. 5F. As shown in FIG. 5G, the portion of imaging device 500 positioned adjacent to facet 501-2 of casing 501 includes components of illumination assembly (e.g., optical filter 502-c-1 coupled to outer wall 522 of chassis 520) and imaging unit 504 surrounded by inner perimeter wall 521. FIG. 5B illustrates optical detectors 510-a (e.g., an array of optical detectors) and lens assembly 506 that will be described in more detail with respect to FIG. 6A. The portion adjacent to facet 501-2 of casing 501 includes components of a power unit (e.g., battery 518-a) and sensor controller 510-b-1.

FIGS. 6A and 6B illustrate views of the internal architecture of example imaging device 600 in accordance with some embodiments of the present disclosure. In some embodiments, imaging device 600 corresponds to imaging device 500 described above with respect to FIGS. 5A-5G. FIGS. 6A and 6B further illustrate components for imaging unit 604 corresponding to imaging unit 104 described above with respect to FIG. 1. Imaging unit 604 is positioned at a center of chassis 620 so that inner perimeter wall 621 surrounds imaging unit 604 at all directions. In FIG. 6A, imaging unit 604 is configured in a circular shape and inner perimeter wall 621 also has a circular cross-section. Imaging unit 604 is configured to receive light reflected (or backscattered) off of an ROI (e.g., ROI 103 in FIG. 1) and collect detector outputs of the ROI (e.g., images).

In some embodiments, imaging unit 604 includes lens assembly 606, filter elements 608, and optical detector 610. In some embodiments, filter elements 608 and lens assembly 606 are positioned relative to optical detector 610 so that light reflected off an ROI propagates through filter elements 608 and lens assembly 606 prior to being incident on optical detector 610. In some embodiments, filter elements 608 are positioned between optical detector 610 and lens assembly 606, as shown in FIG. 6A. In some embodiments, lens assembly 606 is positioned between filter elements 608 and optical detector 610.

In FIG. 6A, lens assembly 606 and filter elements 608 are shown separately from inner perimeter wall 621 for illustrative purposes but are in fact coupled with inner perimeter wall 621, as is shown in FIG. 6B. Lens assembly 606 is configured to receive light reflected off of an ROI (e.g., lens assembly 606 is a lens) and redirect the light (e.g., converge or focus the light) toward optical detectors 610. In some embodiments, lens assembly 606 is a lens. In some embodiments, lens assembly 606 includes one or more lenses. In some embodiments, lens assembly 604 includes a plurality of lenses arranged in an array (e.g., an array including at least 2×2 array of lenses). In some embodiments, the plurality of lenses in the array is arranged in a rectangular or circular configuration.

In some embodiments, lens assembly 604 further includes, or is coupled with, other optical components (e.g., polarizers or anti-reflective coatings). In some embodiments, the lens assembly 604 includes one or more polarizers. In some embodiments, the one or more polarizers are positioned adjacent to the one or more lenses such that the one or more lenses receive light from the ROI through the one or more polarizers. In some embodiments, optical filter elements 608-a are positioned between the one or more lenses of lens assembly 604 and the one or more polarizers of lens assembly 604. In some embodiments, optical filters 608-a are positioned between the one or more polarizers and the one or more lenses and the one or more lenses are positioned between filters 608-a and optical detector 610. The one or more polarizers are configured to transmit light having a particular polarization while blocking (e.g., reflecting or absorbing) light having a polarization distinct from the particular polarization. For example, the one or more polarizers are configured to transmit light having a first linear polarization (e.g., s- or p-polarized light) while blocking light having a polarization distinct from the first linear polarization. In some embodiments, the one or more polarizers are diode polarizing filters. The one or more polarizers may reduce or eliminate detection of stray light reflections from off-axis imaging directions. In some embodiments, lens assembly 606 includes an array of polarizers. For example, lens assembly includes an array of polarizers and an array of lenses, such that each lens of the array of lenses is coupled with a respective polarizer of the array of polarizers.

As noted above, in some embodiments imaging device 600 includes filter elements 608-a-n (e.g., filter elements 608-a-1 and 608-a-2). In some embodiments, filter elements 608-a are mechanically coupled to form a filter assembly or an array of filters. A respective filter element of filter elements 608-a-n is configured to transmit light having a particular wavelength range while blocking (e.g., reflecting and/or absorbing) light having a wavelength outside the particular wavelength range. In some embodiments, filter elements 608-a are bandpass filters. In some embodiments, filter elements 608-a are positioned between optical detectors 610-a and lens assembly 606. Thus, filter elements 608-a are configured to filter the light that is ultimately incident on optical detectors 610-a (e.g., light reflected off of an ROI of an object).

As described above with respect to FIG. 5A, in some embodiments light source elements 602-a output light with distinct wavelength ranges sequentially while imaging unit 604 captures images of an region of interest sequentially. In such embodiments, filter elements 608-a may be omitted.

In some embodiments, imaging unit 604 further includes optical detector 610 including a plurality of optical detector elements 610-a (e.g., detector elements 610-a-1 and 610-a-2). In some embodiments, detector elements 610-a are photodiodes. In some embodiments, the plurality of detector elements 610-a is arranged in an array. For example, the plurality of detector elements 610-a are arranged in a rectangular array (e.g., a square array) or in a circular array. Optical detector 610 is configured to capture detector outputs of a region of interest illuminated with illumination assembly 602 so that each detector element 610-a captures a detector output of the substantially same region. In some embodiments, detector elements 610-a capture detector output sequentially. In some embodiments, detector elements 610-a capture detector outputs concurrently. In some embodiments, the detector outputs are images including spatially resolved data of light received from the ROI (e.g., each optical detector element includes an array of detector pixels). In some embodiments, the detector outputs are values corresponding to light intensity without providing spatially resolved data (e.g., each optical detector element is a single photodiode).

In some embodiments, each filter element 608-a is optically coupled with a single detector element 610-a. For example, in FIG. 6A filter element 608-a-1 is configured to be optically coupled with detector element 610-a-1 without being optically coupled with any other optical detector (e.g., filter element 608-a-1 is not in optical communication with detector element 610-a-2). In some implementations, each filter element 608-a is configured to have a different passband (e.g., each filter element 608-a is configured to transmit light with a different wavelength range, termed a band-pass range herein). Accordingly, even though detector element 610-a-1 collect light from the same ROI, each optical detector captures a different spectral component in such embodiments. For example, as discussed in greater detail herein, filter element 608-a-1 has a passband centered around 520 nm and filter element 608-a-1 has a passband centered around 540 nm. Thus, imaging device 600 captures an exposure, detector element 610-a-1 (which is filtered by filter element 608-a-1) will capture a detector output representing the portion of the incoming light having a wavelength centered around 520 nm, and detector element 610-a-2 (which is filtered by filter element 608-a-2) will capture a detector output representing the portion of the incoming light having a wavelength around 540 nm. As used herein, the term exposure refers to a single detection operation that results in the concurrent or substantially concurrent capture of multiple detector outputs on multiple photo-sensors. These detector outputs, along with the other detector outputs captured by optical detector elements 610-a (each capturing a different spectral band), are then assembled into a hyperspectral data cube for further analysis.

In some implementations, at least a subset of filter elements 608-a is configured to allow light corresponding to two (or more) discrete spectral bands to pass through the filter. While such filters may be referred to herein as dual bandpass filters, this term is meant to encompass bandpass filters that have two discrete passbands as well as those that have more than two discrete passbands (e.g., triple-band bandpass filters, quadruple-band bandpass filters, etc.). By using bandpass filters that have multiple passbands, each photo-sensor can be used to capture detector outputs representing several different spectral bands. For example, imaging device 600 will first illuminate an object with light within a spectral range that corresponds to only one of the passbands of each of the bandpass filters and capture an exposure under the first lighting conditions. Such illumination is accomplished by either having light source elements 602-a that emit light with different wavelength ranges or by having optical filters 602-c coupled with distinct light source elements 602-a, as explained above with respect to FIG. 5A. Subsequently, imaging device 600 will illuminate an object with light within a spectral range that corresponds to a different one of the passbands on each of the bandpass filters, and then capture an exposure under the second lighting conditions. Thus, because the first illumination conditions do not include any spectral content that would be transmitted by the second passband, the first exposure results in each photo-sensor capturing only a single spectral component of the reflected light. Conversely, because the second illumination conditions do not include any spectral content that would be transmitted by the first passband, the second exposure results in each photo-sensor capturing only a single spectral component of the reflected light.

In some embodiments, optical filters 602-c and/or optical filters 608-a include passbands centered at a wavelength selected from 520 nm, 540 nm, 560 nm, 580 nm, 590 nm, 610 nm, 620 nm, and 640 nm. In some embodiments, optical filters 608-a each include a first passband falling within the range of 500-585 nm, and a second passband falling within the range of 585-650 nm. In some embodiments, optical filters 608-a each include a first wavelength range centered at a wavelength selected from 520 nm, 540 nm, 560 nm, and 580 nm and a second wavelength range centered at a wavelength selected from 590 nm, 610 nm, 620 nm, and 640 nm. In some embodiments, optical filters 602-c and/or optical filters 608-a include passbands centered at a wavelength selected from 520 nm, 540 nm, 560 nm, 580 nm, 590 nm, 610 nm, 620 nm, and 660 nm. Further description of example sets of wavebands useful for tissue oximetry is provided, for example, in U.S. Pat. No. 10,010,278, the disclosure of which is expressly incorporated herein by reference, in its entirety, for all purposes.

FIG. 6B illustrates imaging device 600 with imaging unit 604 including optical detector 610, filter elements 608-a, and lens assembly 606 positioned adjacent to each other and coupled with inner perimeter wall 621 of chassis. As shown, imaging unit 604 is coupled with inner perimeter wall 631 so inner perimeter wall 631 extends over imaging unit 604 in a direction substantially parallel to geometric axis 520-1 of chassis 620, as described above with respect to FIG. 5A. In some embodiments, geometric axis 620-1 substantially corresponds to an optical axis of imaging unit 604.

FIG. 6C illustrates a cross-sectional view of example imaging device 600 illuminating a region of interest, in accordance with some embodiments of the present disclosure. The cross-sectional view is presented along a reference line CC′ shown in FIG. 6B. As shown, light source elements 602-a-1 and 602-a-2 are configured to emit light 602-d toward ROI 603. Inner peripheral wall 621 is positioned around imaging unit 604. Inner peripheral wall 621 blocks a portion of light 602-d from light source elements 602-a-1 and 602-a-2 thereby preventing that light from entering imaging unit 604. Light 602-e backscattered from ROI 603 is received by imaging unit 604. The configuration of imaging device allows for illumination of ROI 603 substantially uniformly at a short distance (e.g., distance A1 in FIG. 5D) while preventing at least a portion of stray light emitted by light source elements 602-a-1 and 602-a-2 from entering imaging unit 604 by inner perimeter wall 621. Such configuration improves signal to noise ratio thereby enhancing image quality. Such feature has significance in being able to collect hyperspectral images using low illumination power.

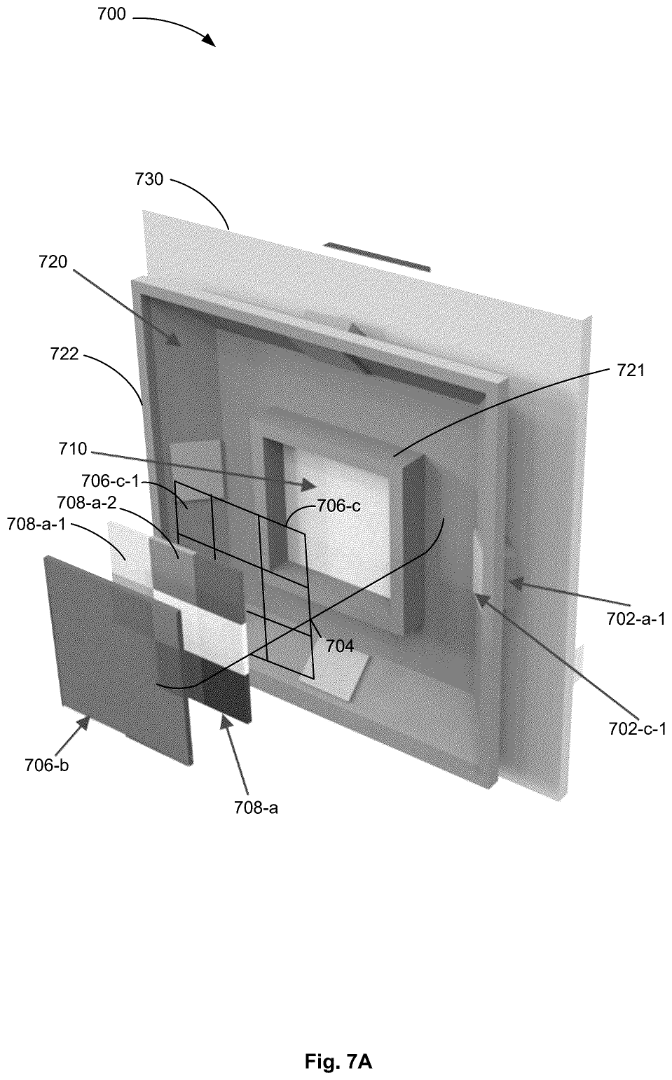

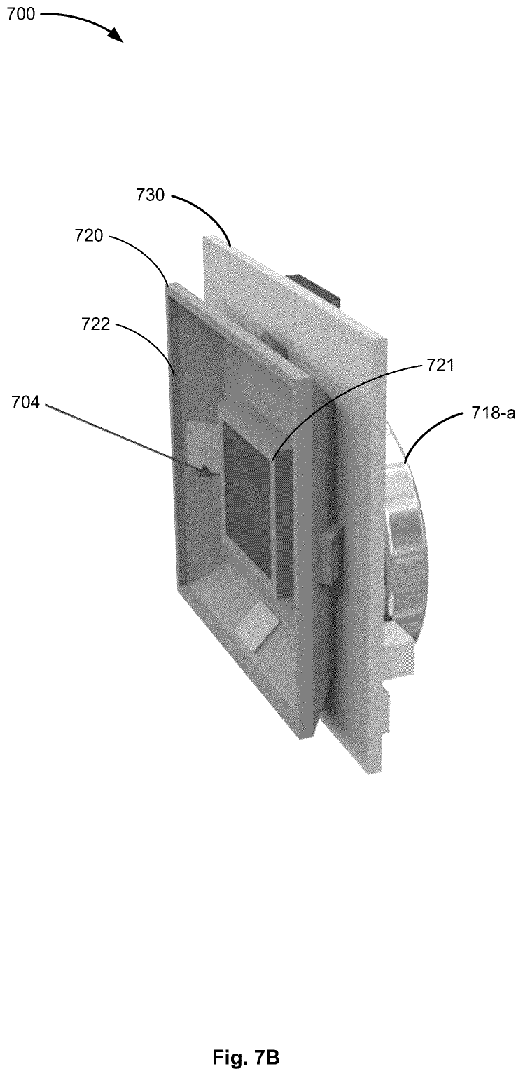

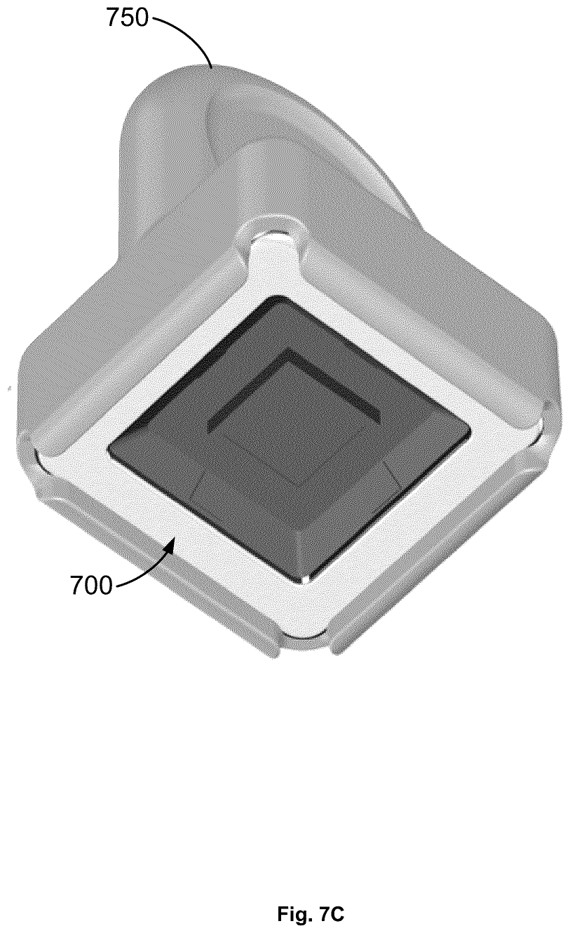

FIGS. 7A and 7B illustrate views of the internal architecture of an example imaging device 700 in accordance with some embodiments of the present disclosure. In some embodiments, imaging device 700 corresponds to imaging device 600 described above with respect to FIGS. 6A and 6B, except that imaging device 700 chassis 720 including outer wall 722 and inner perimeter wall 721 having a square shape (e.g., as shown with alternative outer wall 523 in FIG. 5A). As shown, also imaging unit 704 is also configured having a square shape.

In some embodiments, optical detector 710 includes one or more photodiodes, a charge-coupled device (CCD), an active-pixel sensor (APS), complementary metal-oxide-semiconductor (CMOS), or any combination thereof. Optical detector 710 is thereby configured to capture individual images having a spatial resolution. As shown, in such embodiments, optical detector 710 is coupled with filter 708-a including a plurality of filter regions 708-a (e.g., filter regions 708-a-1 and 708-a-2). Filter regions 708-a have distinct passbands, as described above with respect to filter elements 608-a. Thus, distinct regions of optical detector 710 receive light filter by filter regions 708-a having distinct bandpass regions thereby obtaining a plurality of image portions with distinct spectral information. Such portions are thereby used for forming hyperspectral images. In some embodiments, a respective portion of optical detector 710 receiving light filtered by a respective filter region 708-a corresponds to a plurality of pixels (e.g., an array or a cluster of CCD pixels, APS pixels, or CMOS pixels). For example, a respective portion of optical detector 710 corresponds to an array of 2×2 pixels, 3×3 pixels, 4×4 pixels, 5×5 pixels, 6×6 pixels, etc., an array of 4×3 pixels, 8×6 pixels, or 16×12 pixels, etc., or an 8×5 pixels, 16×10 pixels, or 24×15 pixels, etc.

As shown in FIG. 7A, in some embodiments, optical detector 710 is coupled with lens 706-c including a plurality of lens elements (e.g., lens element 608-c-1). For example, lens 706-c includes an array of 3×3 lens elements. In some embodiments, each lens element 706-c is in optical communication with a single filter region (e.g., lens element 706-c-1 is in optical communication with filter region 708-a-1) without being in optical communication with any other filter region of filter 708-a. Consequently, each lens element 706-c is in optical communication with a respective portion of optical detector 710. For example, light transmitted through filter region 708-a-1 is redirected (e.g., focused) by lens element 706-c-1 toward a respective portion of optical detector 710.

As explained above, in some embodiments, imaging unit 704 includes one or more polarizers (e.g., polarizer 706-b shown in FIG. 7A). In some embodiments, polarizer 706-b is a diode polarizing filter configured to transmit light having a particular polarization while blocking (e.g., absorbing) light having a polarization distinct from the particular polarization. For example, illumination assembly 702 includes a plurality of polarizers configured to transmit light having a first linear polarization (e.g., filter element 702-c-1 includes a polarizer). Each of these polarizers is coupled with a respective light source element (e.g., light source element 702-a-1) so that light incident on an ROI (e.g., ROI 103 in FIG. 1) has the first linear polarization. The light reflected off of the ROI also has the first linear polarization while light that has interfaced with the ROI is mostly unpolarized (e.g., the light forming a hyperspectral image is unpolarized). Polarizer 706-b of imaging unit 704 is configured to block light having the first linear polarization. The light reflected off of the ROI is thereby blocked by polarizer 706-b while at least a portion of the unpolarized light that has interacted with the ROI is transmitted through polarizer 706-b toward optical detector 710. Optical detector 710 therefore only receives light that has interacted with the ROI.

In some embodiments, filter 708-a is omitted. Instead, illumination assembly 704 includes a plurality of optical filters 702-c (e.g., optical filter 702-c-1). Each optical filter 702-c is in optical communication with a respective light source element 702-a (e.g., light source element 702-a-1). In some embodiments, optical filter 702-c-1 and light source element 702-a-1 correspond to optical filter 702-c-1 and light source element 702-a-1 described above with respect to FIG. 5A. In such embodiments, light source elements 702-a emit light sequentially so that a set of light source elements (e.g., one or more light source elements) that are configured to transmit light with a particular wavelength range emit at the same time. For example, a first set of light source elements 702-a emitting light having a first wavelength range is configured to illuminate an ROI at a first exposure time while optical detector 710 captures a first image. A second set of light source elements 702-a emitting light having a second wavelength range distinct from the first wavelength range is configured to illuminate the ROI at a second exposure time, distinct and exclusive from the first exposure time, while optical detector 710 captures a second image. The first image and the second image are used to form a hyperspectral image of the ROI.

FIG. 7B illustrates an alternative view of the interior of imaging device 700. FIG. 7B illustrates chassis 720 with inner perimeter wall 721 and outer wall 722. Chassis 720 is coupled with board 730. As shown, imaging unit 704 is surrounded by inner perimeter wall 721. FIG. 7B also illustrates battery 718-a positioned coupled with board 730.

FIG. 7C illustrates the example device of FIGS. 7A and 7B in example handheld housing 750, in accordance with some embodiments of the present disclosure. In FIG. 7C, imaging device 700 coupled with handheld enclosure 750. In some embodiments, handheld enclosure 750 corresponds to handheld enclosure 350 described with respect to FIG. 3.

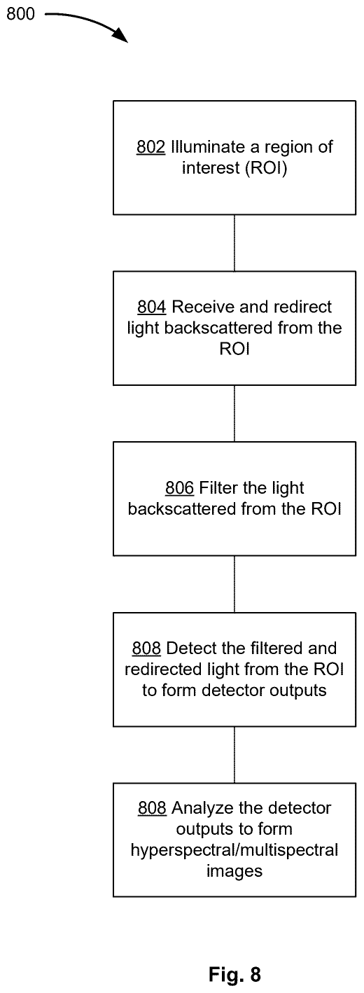

FIG. 8 illustrates a block diagram of method 800 of collecting and generating hyperspectral image data, in accordance with some embodiments of the present disclosure. Method 800 is performed with any of the imaging devices $00 described above with respect to FIGS. 1-7C. Method 800 includes illuminating (block 802) a region of interest (ROI) (e.g., ROI 103 in FIG. 1) with a light source (e.g., a light source of illumination assembly 102). The method includes receiving and redirecting (block 804) light backscattered from the ROI by a lens (e.g., lens assembly 106 of imaging unit 104). The method includes filtering (block 806) the light backscattered by the ROI by a filter including one or more filters or filter regions (e.g., filter unit 108). Method 800 also includes detecting (block 808), the light backscattered from the ROI, after the light has passed through the lens and the filter, by an optical detector (e.g. photo-sensor unit 110), thereby forming detector outputs (e.g., images) of the ROI. The detector outputs include one or more spectral signatures associated with the ROI. Method 800 further includes analyzing (block 810) the detector outputs to form hyperspectral images of the ROI.

In some implementations, the imaging devices $00 described above with respect to FIGS. 1-7C can be used in communication with a distributed client-server system. A distributed client-server system and accompanying methods allow for both spectral and spatial resolution at low cost, owing to the division of hardware and computational requirements for imaging (e.g., with a low cost hyperspectral/multispectral camera) and image processing (e.g., using one or more remote servers). Examples of distributed client-server system and accompanying methods are described in U.S. Patent Application Publication No. 2015/0142461, the content of which is expressly incorporated by reference herein, in its entirety, for all purposes.

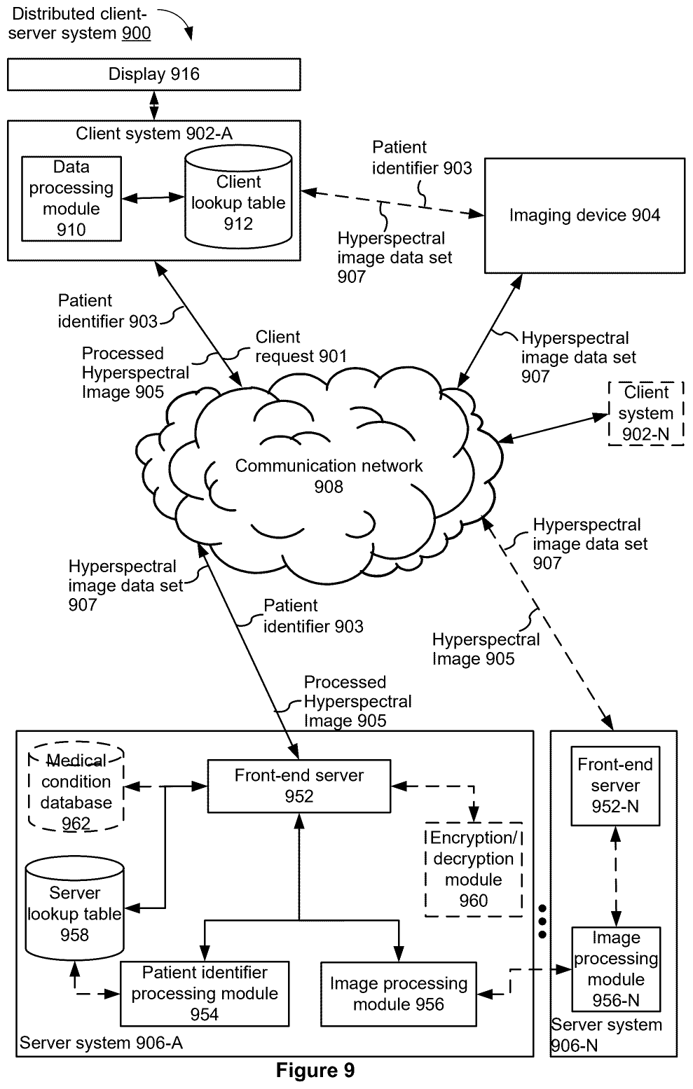

FIG. 9 is a block diagram illustrating distributed client-server system 900 for processing hyperspectral images via a computer network, in accordance with some implementations.

In some implementations, the distributed client-server system 900 includes one or more client systems 902 (“clients 902,” e.g., client 902-A . . . client 902-N), an imaging device 904, one or more server systems 906 (“servers 906,” e.g., server 906-A . . . server 906-N), and a communication network 908. In some embodiments, imaging device 904 corresponds to any of the imaging devices $00 described with respect to FIGS. 1-7C.

In some embodiments, client system 902-A corresponds to a system operated by a physician or a healthcare provider whereas imaging device 904 is operated by the patient (e.g., the patient is performing measurements as described with respect to FIGS. 3 and 4). In some embodiments, client system 902-A corresponds to a system operated by the patient (e.g., the patient is performing measurements and self-monitoring his or her condition). In some embodiments, client system 902-A corresponds to a system operated by a patient while another client system of client systems 902-N is operated by a physician. For example, the patient collects imaging data with imaging device 904 which is communicated to client system 902-A and client system 902-N to be reviewed by the patient as well as the physician.

In some embodiments, client system 902-A is a mobile device (e.g., a smart phone, a laptop, or a tablet computer) including an application associated with imaging device 904. The application allows the user of client system 902-A to review hyperspectral images and/or parameters derived based on the hyperspectral images (e.g., oxygen saturation level and/or oximetry index value). In some embodiments, the application allows a patient to self-monitor his or her condition, For example, the application may provide an alert to indicate to the patient when the condition is worsening.

In some implementations, client 902 sends a client request (e.g., a client request 901), requesting a unique patient identifier from the server system 906. In some implementations, after sending the client request, the client 902 receives a unique patient identifier (e.g., a patient identifier 903) from the server 906 (via communication network 908). In some implementations, the client 902 receives a processed hyperspectral image (e.g., a processed hyperspectral image 905) from the server 906.

In one embodiment, a clinician, or medical establishment (e.g., clinic, hospital, HMO, or PPO) with which the clinician is associated, purchases a limited or unlimited-use contract for processing of a plurality of hyperspectral image data sets over a given period of time (e.g., 1 month, 2 months, 3 months, 4 months, 6 months, 12 months, or 24 months). For example, in one implementation, a clinician or medical establishment purchases a license granting use of an unlimited number of processing events over a twelve month period. In another implementation, a clinician or medical establishment pre-purchases a set number of processing events at a pre-determined price (e.g., 900 processing events at X dollars each).

In other embodiments, the clinician or medical establishment is charged on a per use basis, e.g., is charged every time a processing event is requested. For example, the clinician or medical establishment requesting processing of a hyperspectral image data set is charged a one-time fee for processing of the unique data set. Accordingly, in some implementations, in response to receiving a request for a unique patient identifier, or upon receiving a hyperspectral image data set 907, from the imaging device 904, the server 906 sends a fee request to the client 902 (e.g., an invoice) or a credit source associated with the client 902 (e.g., a credit card, PAYPAL, etc.). In one implementation, where a clinician or medical establishment is billed on a fixed schedule, e.g., monthly or bi-annually, in response to receiving a request for a unique patient identifier, or upon receiving a hyperspectral image data set 907, from the imaging device 904, the server 906 creates a billing event on an internal log, which is included on a fee request (e.g., invoice) at the pre-determined time of billing.

In some implementations, the client 902 includes a data processing module 910, and a client lookup table 912. In some implementations, the data processing module 910 associates a processed hyperspectral image 905 with patient identity in accordance with the client lookup table 912. In some implementations, the data processing module 910 provides additional processing to a processed hyperspectral image received from the server 906, such as image contrast or size adjustment, zoom-in or zoom-out, and rotation. In some implementations, the client lookup table 912 includes patient information (e.g., a patient's name, date of birth, home address, and/or social security number), one or more unique patient identifiers (e.g., a randomly or pseudo-randomly generated identifier that uniquely identifies a patient), and one or more mappings there between. In some implementations, the client 902 is connected to a display 914 (e.g., a computer monitor or projector). In some implementations, the client 902 is a desktop computer, a laptop, a pad type device, or a smart phone. In some embodiments, client 902 corresponds to user device 460 described with respect to FIG. 4.

In some implementations, the imaging device 904 illuminates an object (e.g., ROI 103 described with respect to FIG. 1) and generates imaging data (e.g., detector outputs) of the object, as described above with respect to imaging device 100 in FIG. 1.

In some implementations, after a plurality of images is generated, the imaging device 904 generates imaging data cube (e.g., the hyperspectral image data set 907) in accordance with the generated images. In some embodiments the plurality of images is sent to the server system 906 to form an imaging data set and processed hyperspectral image 905. In some embodiments, processed hyperspectral 902 is further analyzed to determine parameters associated with the image. In some embodiments, the parameters include an oxygen saturation value or an oximetry index value based on a concentration values of one or more spectral signatures in the hyperspectral image. In some embodiments the plurality of images and/or parameters is sent to the client system 902 to form an imaging data set and processed hyperspectral image 905. In some implementations, the hyperspectral image data set 907 prepared by the imaging device 904 is transmitted to one or more servers 906 for further processing (e.g., forming the processed hyperspectral image 905 or comparison to a medical condition database 962). In other implementations, the hyperspectral image 905 is formed by the imaging device 904.

In some implementations, the plurality of images is sent to the client system 902 for pre-processing (e.g., compression of the images, encryption, or creation of the hyperspectral image data set 907). In some implementations, the client system sends the pre-processed images or hyperspectral image data set 907 to one or more servers 906 for further processing (e.g., forming the hyperspectral image 905 or comparison to a medical condition database 962). In certain embodiments, where the imaging device sends the plurality of images to the client system 902-A-1, associated with a first institution, the server 906 transmits processed data (e.g., hyperspectral image 905, a diagnosis, or a prognosis) to a second client system 902-A-2, associated with the same institution.

In some implementations, the server 906 (i) generates a unique patient identifier (e.g., the patient identifier 903) in response to a request from the client 902, and (ii) processes a hyperspectral image data set (e.g., the hyperspectral image data set 907 or images) received from the imaging device 904 to form a hyperspectral image (e.g., the hyperspectral image 905). In some implementations, the server 906 includes a frontend server 952, a patient identifier processing module 954, an imaging processing module 956, a server lookup table 958, optionally, an encryption/decryption module 960, and, optionally, a medical condition database 962. In some implementations, the frontend server 952 receives and relays client requests to corresponding modules within the server 906. In some implementations, the frontend server 952 also transmits the patient identifier 903 and the corresponding processed hyperspectral image 905 to the client 902 for review by a physician. In some implementations, the patient identifier processing module 954 generates a patient identifier 903 in response to a request from the client 902. In some implementations, the patient identifier processing module 954 also stores in the server lookup table 958 (i) the unique patient identifier 903 and (ii) information identifying a specific client (e.g., the client 902-A) among a plurality of clients 902. In some implementations, the imaging processing module 956 processes the hyperspectral image data set 907 received from the imaging device 904 to form a processed hyperspectral image 905.

In some implementations, the server lookup table 958 includes one or more mappings between unique patient identifiers and information identifying respective clients 902. In some implementations, the encryption/decryption module 960 decrypts an encrypted hyperspectral image data set for processing by the image processing module 956, or encrypts a hyperspectral image generated by the image processing module 956 before the hyperspectral image is transmitted to the client 902.

In some implementations, the medical condition database 962 includes one or more hyperspectral signatures, which correspond to one or more medical conditions, for comparison with a processed hyperspectral image 905 generated by the image processing module 956.

In some implementations where the distributed client-server system 900 includes a plurality of clients 902 (e.g., the client 902-A . . . , and the client 902-N), the server 906 identifies a specific client 902 (e.g., the client 902-A) among a plurality of clients 902, in accordance with the unique patient identifier associated with a hyperspectral image data set, and the server lookup table 958. In some implementations, after the identification process, the server 906 transmits the hyperspectral image formed based on the hyperspectral image data set, to the identified client 902. In some implementations, after the identification process, the server 906 transmits a diagnosis, probability of each of one or more diagnosis, or likelihood or odds of each of one or more diagnosis, based on a comparison between the hyperspectral image data set 907 or processed hyperspectral image 905 and the medical condition database 962, to the identified client 902. In yet other implementations, the server 906 transmits a processed hyperspectral image 905 and a diagnosis, probability of one or more diagnosis, or prognosis for one or more treatments to the identified client 902. In other implementations, the server 906 transmits the processed hyperspectral image 905 to one or more clients 902 that share a predefined relationship with the identified client 902. For example, in some implementations, the server 906 transmits the processed hyperspectral image 905 to clients 902 that are within a same local area network (e.g., an internal network owned by a same hospital or clinic) as the identified client 902. As another example, in some implementations, the server 906 transmits the processed hyperspectral image 905 to clients 902 that are registered as belonging to a same physician as the identified client 902. As still another example, in some implementations, the server 906 transmits the hyperspectral image to clients 902 that belong to persons given authorization by the treating physician to review the hyperspectral image, such as physician assistants who work under the supervision of the treating physician. This approach provides flexibility but still protects patient privacy. In this approach, a hyperspectral image 905 indicative of a patient's medical condition is sent to one or more computers, when these computers are under control of a same treating physician, thereby minimizing the risk of breaching patient confidentiality by an unauthorized third party.

In some implementations where the distributed client-server system 900 includes a plurality of servers 906 (e.g., the server 906-A . . . , and the server 906-N), one server 906 (e.g., the server 906-A) communicates with another server 906 (e.g., the server 906-N). In some implementations, the image processing module 956 of one server 906 communicates with the image processing module 956 of another server 906. In some implementations, the imaging device 904 transmits different portions of a hyperspectral image data set to different servers 906—e.g., a first portion of the hyperspectral image data set 907 to the server 906-A, and a second portion (e.g., different from the first portion) of the hyperspectral image data set 907 to the server 906-N—for parallel processing. In some implementations, where redundancy is desired, the imaging device 904 transmits the same portion of a hyperspectral image data set to two or more servers 906 (with different computational power)—e.g., the same portion of the hyperspectral image data set 907 to both the server 906-A and the server 906-N—and results from the server 906 that finishes the processing earliest are used to form the hyperspectral image.

In some implementations, the communication network 908 optionally includes the Internet, one or more location connections, one or more local area networks (LANs), one or more wide area networks (WANs), other types of networks, or a combination of such networks. In some implementations, the one or more location connections optionally include connections by infrared signals, radio frequency signals, local area networks (LANs), Bluetooth, serial or parallel cable, or a combination of thereof. In some embodiments, the imaging device 904 is connected to the communication network 908 through a wireless 802.99 device. In some embodiments, the imaging device 904 is in direct electronic communication with the system server 906 through a wireless 802.11 device. In such instances, the wireless 802.11 device is construed as the communication network 908.

In some embodiments, the imaging device 904 includes a docking port and is configured to be docked directly onto the server system 906. In such embodiments, the docking interface between the imaging device 904 and the server system 906 is construed as the communication network 908.

### Description of Embodiments

In light of these descriptions, we now turn to embodiments of hyperspectral imaging devices.

In some aspects, the disclosure provides hyperspectral imaging devices suitable for use outside of a clinical setting, e.g., within a patient's home. In some embodiments, as described herein, this is realized by arranging the optical components of the imaging device around a chassis (e.g., chassis 520 as illustrated in FIG. 5) having an inner perimeter wall (e.g., inner perimeter wall 521 as illustrated in FIG. 5), within which an imaging unit (e.g., imaging unit 504) is disposed, and an outer wall (e.g., outer wall 522), upon which an illumination assembly (e.g., illumination assembly 102) is disposed. The outer wall is angled away from the inner perimeter wall, such that light irradiating from the illumination assembly can illuminate a region of interest, e.g., on the skin of an individual. The inner perimeter wall has a height that is sufficient to block stray light irradiated from the illumination assembly from reaching the imaging unit disposed within (e.g., light from light source 502-a-1 in FIG. 5 is blocked from reaching imaging unit 504 by inner perimeter wall 521). Among other advantages, this configuration facilitates imaging at close distances, using low light output from the illumination system, such that the power requirements of the system are low enough to be powered by simple, low voltage batteries.

For example, in one aspect, the disclosure provides an imaging device that includes a chassis (e.g., chassis $20) having a first side facing an exterior, a second side facing away from the exterior, and an axis (e.g., axis $20-1) pointing from the second side to the first side. The chassis includes an inner, e.g. circumferential, perimeter wall (e.g., wall $21) extended substantially along the axis of the chassis from the second side toward the first side of the chassis, e.g., defining a first area within the inner perimeter wall. The chassis also includes an oblique outer, e.g., circumferential, perimeter wall (e.g., wall $22) extended at an acute angle with respect to the axis of the chassis from the second side toward the first side of the chassis, e.g., defining a second area between the outer wall and the inner perimeter wall.

The device also includes a light source (e.g., light source $02-a, which is part of illumination assembly $02) attached to or disposed at the oblique outer wall (e.g., wall $22) of the chassis. The light source is configured to illuminate a region of interest (ROI) (e.g., ROI 103 in FIG. 1). In some embodiments, the light source includes a plurality of light source elements.

The device also includes a lens (e.g., lens assembly $06) attached to or disposed within the chassis, e.g., within the first area defined by the inner perimeter wall of the chassis (e.g., wall $21), and configured to collect light backscattered by the ROI. In some embodiments, the lens includes a plurality of lens elements. For example, in some embodiments, lens assembly $06 includes an array of lens elements (e.g., lens 706-c includes an array of lens elements 706-c-n), each of which is in optical communication with a different color filter element 708-a-n in optical filter 708-a, as illustrated in FIG. 7A.

The device also includes an optical filter (e.g., filter $08-a, which is part of filter unit $08) attached to or disposed within the chassis, e.g., within the first area defined by the inner perimeter wall (e.g., wall $21) of the chassis, and configured to filter the light collected by the lens (e.g., lens assembly $06). In some embodiments, the optical filter is configured to receive the light backscattered by the ROI after the light is collected by the lens (e.g., FIG. 6A). In some embodiments, the optical filter is configured to filter the light backscattered by the ROI prior to being collected by the lens (e.g., FIG. 7A). The optical filter includes a plurality of optical filter elements. Each respective optical filter element in the plurality of optical filter elements is in optical communication with the lens, allowing light, e.g., reflected of backscattered by the ROI, of a corresponding spectral range in a plurality of spectral ranges to pass while blocking light of any other spectral range in the plurality of spectral ranges.

The device also includes an optical detector (e.g., detector $10-a, which is part of photo-sensor unit $10) attached to or disposed within the chassis, e.g., within the first area defined by the inner perimeter wall (e.g., wall $21) of the chassis, in optical communication with the optical filter (e.g., filter unit $08) and configured to resolve light filtered by the optical filter. The optical detector includes a plurality of optical detector elements (e.g., optical detector elements $10-a-n) and each respective optical detector element in the plurality of optical detector elements is covered by a corresponding optical filter element in the plurality of optical filter elements (e.g., in filter unit $08), e.g., thereby capable of generating a plurality of detector outputs.

The device also includes a control system to control the operation of the light source and the optical detector. The optical detector (e.g., detectors $10-a) is surrounded by the inner perimeter wall of the chassis (e.g., wall $21). The inner perimeter wall of the chassis (e.g., wall $21) has a height along the axis of the chassis to block stray light of the light source (e.g., light source $02-a) from reaching the plurality of optical detectors (e.g., detector $10-a).

In some embodiments, the control system (e.g., control system 114 in FIG. 1) includes one or more processors, memory, and one or more programs stored in the memory and configured to be executed by the one or more processors. The one or more programs include instructions for turning on the light source to illuminate the ROI, and using the optical detector to collect light, thereby generating a plurality of detector outputs. In some embodiments, the one or more programs stored in the memory further include instructions for performing a spectral analysis on the plurality of detector outputs to determine a concentration value of each respective spectral signature in one or more spectral signatures associated with the ROI. In other embodiments, spectral analysis is performed on a separate device, e.g., a personal electronic device or within a cloud server.

In some embodiments, the optical detector includes one or more photodiodes, a charge-coupled device (CCD), a complementary metal-oxide-semiconductor (CMOS), an active-pixel sensor (APS), or any combination thereof. In some embodiments, each respective optical detector element in the plurality of optical detector elements includes a single photodiode (e.g., detector elements 610-a-n are photodiodes in FIG. 6A). In some embodiments, each respective optical detector element in the plurality of optical detector elements includes a single pixel or a cluster or an array of pixels of the CCD, CMOS, or APS (e.g., optical detector 710 includes an CCD, CMOS, or APS in FIG. 7A).

In one aspect, the disclosure provides a device including a chassis (e.g., chassis $20) having a first side facing an exterior, a second side facing away from the exterior, and an axis pointing from the second side to the first side. The chassis includes an inner, e.g., circumferential, perimeter wall (e.g., inner perimeter wall $21) extended substantially along the axis of the chassis from the second side toward the first side of the chassis, e.g., defining a first area within the inner perimeter wall. The chassis also includes an oblique outer, e.g., circumferential, perimeter wall (e.g., outer wall $22) extended at an acute angle with respect to the axis of the chassis from the second side toward the first side of the chassis, e.g., defining a second area between the outer wall and the inner perimeter wall.

The device also includes a light source (e.g., light source $02-a, which is part of illumination assembly $02) attached to or disposed at the oblique outer wall (e.g., wall $22) of the chassis. The light source is configured to illuminate a region of interest (ROI). In some embodiments, the light source includes a plurality of light source elements.

The device also includes a lens (e.g., lens assembly $06) attached to or disposed within the chassis, e.g., within the first area defined by the inner perimeter wall of the chassis (e.g., wall $21), and configured to collect light backscattered by the ROI. In some embodiments, the lens includes a plurality of lens elements.

The device also includes an optical filter (e.g., filter $08-a, which is part of filter unit $08) attached to or disposed within the chassis, e.g., within the first area defined by the inner perimeter wall (e.g., wall $21) of the chassis, in optical communication with the lens (e.g., lens assembly $06) and configured to filter the light collected by the lens, e.g., which was backscattered from the ROI. The optical filter includes an array of filter regions, each filter region in the array of filter regions comprises a plurality of optical filter elements. Filter arrays for hyperspectral imaging are described, for example, in U.S. Pat. No. 9,766,382, the disclosure of which is expressly incorporated herein by reference, in its entirety, for all purposes. Each respective optical filter element in the plurality of optical filter elements allows light, e.g., backscattered by the ROI, of a corresponding spectral range in a plurality of spectral ranges to pass while blocking light of any other spectral range in the plurality of spectral ranges.

The device also includes an optical detector (e.g., detector $10-a, which is part of photo-sensor unit $10) attached to or disposed within the chassis, e.g., within the first area defined by the inner perimeter wall (e.g., wall $21) of the chassis, and in optical communication with the optical filter (e.g., filter unit $08) and configured to resolve light filtered by the optical filter. The optical detector includes an array of detector regions (e.g., regions of optical detector 710 or regions of a two-dimensional detector array). Each detector region in the array of detector regions includes a plurality of optical detector elements, where each respective optical detector element in the plurality of optical detector elements is covered by a corresponding optical filter element in the plurality of optical filter elements, e.g., thereby capable of generating a hyperspectral data cube of detector outputs from a single image capture.

The device also includes a control system to control operation of the light source and the optical detector. The optical detector (e.g., detectors $10-a) is surrounded by the inner perimeter wall of the chassis (e.g., wall $21). The inner perimeter wall of the chassis (e.g., wall $21) has a height along the axis of the chassis to block stray light of the light source (e.g., light source $02-a) from reaching the plurality of optical detectors (e.g., detector $10-a).

In some embodiments, the control system includes one or more processors, memory, and one or more programs stored in the memory and configured to be executed by the one or more processors. The one or more programs include instructions for turning on the light source to illuminate the ROI, and using the optical detector to collect light, thereby generating a plurality of detector outputs. In some embodiments, the one or more programs stored in the memory further include instructions for performing a spectral analysis on the hyperspectral data cube of detector outputs to determine concentration values of each respective spectral signature in one or more spectral signatures at each respective point in an array of points corresponding to a two-dimensional area of the ROI.

In some embodiments, the optical detector includes one or more photodiodes, a charge-coupled device (CCD), a complementary metal-oxide-semiconductor (CMOS), an active-pixel sensor (APS), or any combination thereof. In some embodiments, for each detector region in the array of detector regions, each respective optical detector element in the plurality of optical detector elements includes a single pixel or a cluster of pixels of the CCD, CMOS or APS.

In one aspect, the disclosure provides a device including a chassis (e.g., chassis $20) having a first side facing an exterior, a second side facing away from the exterior, and an axis pointing from the second side to the first side. The chassis includes a, e.g., circumferential, perimeter wall (e.g., inner perimeter wall $21) extended substantially along the axis of the chassis from the second side toward the first side of the chassis, e.g., defining a first area within the inner perimeter wall. The chassis also includes an oblique outer, e.g., circumferential, perimeter wall (e.g., outer wall $22) extended at an acute angle (e.g., angle A in FIG. 5A) with respect to the axis of the chassis from the second side toward the first side of the chassis, e.g., defining a second area between the outer wall and the inner perimeter wall.

The device also includes a light source (e.g., light source $02-a, which is part of illumination assembly $02) including a plurality of light source elements (e.g., light source elements $02-a-n) attached to or disposed at the oblique outer wall (e.g., wall $22) of the chassis and configured to illuminate a region of interest (ROI).

The device also includes an optical filter (e.g., optical filters $02-c, which is part of illumination assembly $02) including a plurality of optical filter elements (e.g., optical filter elements $02-c-n), where each respective optical filter element in the plurality of optical filter elements covers a corresponding light source element (e.g., optical filter 502-c-1 covers corresponding light source element 502-a-1, as illustrated in FIG. 5A) in the plurality of light source elements, and allows the light of a corresponding spectral range in a plurality of spectral ranges to pass while blocking light of any other spectral range in the plurality of spectral ranges.

The device also includes a lens (e.g., lens assembly $06) attached to or disposed within the chassis, e.g., within the first area defined by the inner perimeter wall (e.g., wall $21) of the chassis, and configured to collect light backscattered by the ROI. In some embodiments, the lens includes a plurality of lens elements. For example, in some embodiments, lens assembly 606 includes an array of lens elements, each of which is in optical communication with a different optical detector element 610-a-n, as illustrated in FIG. 6A. FIG. 6A includes an illustration of optical filter 508-a positioned between lens assembly 606 and optical detector element 610-a-n. FIG. 7A includes an illustration of a lens 706-a (e.g., a lens array) positioned between optical filter 708-a. FIG. 7A further illustrates polarizer 706-b positioned adjacent to optical filter 708-a facing an exterior of imaging device 700.

The device also includes an optical detector (e.g., detector $10-a, which is part of photo-sensor unit $10) attached to or disposed within the chassis, e.g., within the first area defined by the inner perimeter wall (e.g., wall $21) of the chassis, in optical communication with the lens (e.g., lens assembly $06), and configured to resolve the light collected by the lens. The optical detector includes an array of optical detector elements (e.g., regions of optical detector 710 or regions of a two-dimensional detector array). In some embodiments, this configuration is capable of generating an array of detector outputs for each pair of the light source element and the filter element.

The device also includes a control system to control operation of the light source and the optical detector. The optical detector (e.g., detectors $10-a) is surrounded by the inner perimeter wall of the chassis (e.g., wall $21). The inner perimeter wall of the chassis (e.g., wall $21) has a height along the axis of the chassis to block stray light of the light source (e.g., light source $02-a) from reaching the plurality of optical detectors (e.g., detector $10-a).

In some embodiments, the control system includes one or more processors, memory, and one or more programs stored in the memory and configured to be executed by the one or more processors. The one or more programs include instructions for sequentially turning on and off each respective light source element in the plurality of light source elements to illuminate the ROI sequentially, and sequentially using the optical detector to collect light when each respective light source element is turned on, thereby generating a plurality of arrays of detector outputs, each array of detector outputs at a corresponding spectral range in the plurality of spectral ranges, where the plurality of arrays of detector outputs collectively forms a hyperspectral data cube of detector outputs. In some embodiments, the one or more programs stored in the memory further include instructions for performing a spectral analysis on the hyperspectral data cube of detector outputs to determine concentration values of each respective spectral signature in one or more spectral signatures at each respective point in an array of points corresponding to a two-dimensional area of the ROI.

In some embodiments, the optical detector includes one or more photodiodes, a charge-coupled device (CCD), a complementary metal-oxide-semiconductor (CMOS), an active-pixel sensor (APS), or any combination thereof. In some embodiments, each respective optical detector element in the array of optical detector elements includes a single pixel or a cluster of pixels of the CCD, CMOS or APS.

In some embodiments of any of the aspects listed above, a spectral signature in the one or more spectral signatures is oxyhemoglobin, deoxyhemoglobin, melanin, or a combination thereof. In some embodiments, an oxygen saturation value or an oximetry index value is determined based on the concentration values of the one or more spectral signatures.

In some embodiments of any of the aspects listed above, the inner perimeter wall forms a, e.g., cylindrical, structure having a nominal diameter that is between 5 mm and 10 mm, between 10 and 15 mm, or between 15 and 20 mm. In some embodiments, the structure has a circular, oval, or polygonal cross-section. In some embodiments, the height of the inner perimeter wall along the axis of the chassis is less than 25 mm, less than 20 mm, less than 15 mm, less than 10 mm, less than 8 mm, less than 6 mm, or less than 4 mm.

In some embodiments of any of the aspects listed above, the acute angle (e.g., Angle A in FIG. 5D) at which the oblique outer wall is extended with respect to the axis of the chassis is between 15 degrees and 30 degrees, between 30 degrees and 45 degrees, between 45 degrees and 60 degrees, or between 60 degrees and 75 degrees.

In some embodiments of any of the aspects listed above, the oblique outer wall forms a truncated conical structure, where a nominal diameter of the truncated conical structure at the second side of the chassis (e.g., diameter D2 in FIG. 5B) is between 10 mm and 15 mm, between 15 mm and 20 mm, between 20 mm and 25 mm, or between 25 mm and 30 mm, and a nominal diameter of the truncated conical structure at the first side of the chassis (e.g., diameter D1 in FIG. 5B) is between 15 mm and 25 mm, between 25 mm and 35 mm, or between 35 and 45 mm. In some embodiments, the truncated conical structure has a circular, oval, or polygonal cross-section.

In some embodiments of any of the aspects listed above, the oblique outer wall has a height (e.g., height H1 in FIG. 5D) along the axis of the chassis that is less than 14 mm, less than 12 mm, less than 10 mm, less than 8 mm, or less than 6 mm.

In some embodiments of any of the aspects listed above, the light source comprises three, four, five, six, seven, eight, or more than eight light source elements (e.g., light source elements $02-a-n). In some embodiments, the three, four, five, six, seven, eight or more than eight light source elements are uniformly distributed around the axis of the chassis at the oblique outer wall of the chassis.

In some embodiments of any of the aspects listed above, the light source emits near infrared light, visible light, ultraviolet light, or any combination thereof.

In some embodiments of any of the aspects listed above, the each light source element of the light source emits white light having a spectrum between 400 nm and 780 nm.

In some embodiments of any of the aspects listed above, the ROI has a size less than 700 mm², less than 650 mm², less than 600 mm², less than 550 mm², less than 500 mm², less than 450 mm², less than 400 mm², less than 350 mm², less than 300 mm², less than 250 mm², less than 200 mm², less than 150 mm², less than 100 mm², or less than 50 mm².

In some embodiments of any of the aspects listed above, the ROI is located at a range that is less than 50 mm, less than 40 mm, less than 30 mm, less than 25 mm, less than 20 mm, 18 mm, less than 16 mm, less than 14 mm, less than 12 mm, less than 10 mm, less than 8 mm, less than 6 mm, less than 4 mm, or less than 3 mm with respect to the second side of the chassis.

In some embodiments of any of the aspects listed above, each respective optical filter element in the plurality of optical filter elements (e.g., optical filter elements $08-a-n) is a bandpass filter. In some embodiments, each respective optical filter element in the plurality of optical filter elements has a different spectral band than any other optical filter element in the plurality of optical filter elements. In some embodiments, at least two optical filter elements in the plurality of optical filter elements have a same spectral band.

In some embodiments of any of the aspects listed above, the device also includes a first polarizer comprising one or more first polarizer elements (e.g., filter element $02-c), where the first polarizer is disposed in an optical path between the light source (e.g., light source $02-a) and the ROI, and configured to selectively allow light that is substantially limited to a first polarization to pass through. In some such embodiments, the device also includes a second polarizer, e.g., including one or more polarizer elements (e.g., filter element $06-b), where the second polarizer is disposed in an optical path between the ROI and the optical detector (e.g., optical detector $10-a), and configured to selectively allow light that is substantially limited to a second polarization to pass through, where the second polarization is in a different direction from the first polarization.

In some embodiments of any of the aspects listed above, the device also includes a power source (e.g., battery $18-a, which is part of power unit $18) in electrical communication with the light source, the optical detector, and/or the control system to provide electrical power to the light source, the optical detector, and/or the control system. In some embodiments, the power source includes one or more batteries (e.g., battery $18-a). In some embodiments, a battery in the one or more batteries includes a lithium button cell and/or a lithium polymer battery, e.g., having a voltage of less than 10 volts, less than 5 volts, or less than 3 volts.

In some embodiments of any of the aspects listed above, the control system also includes a power regulator (e.g., power regulator $18-b, which is part of power unit $18) that maintains a power supply at a desired level.

In some embodiments of any of the aspects listed above, the control system also includes a communication interface (e.g., communication interface $16, e.g., including a communication element $16-a and/or communication antenna $16-b) for wired or wireless communication with an external device or communication network. In some embodiments, the detector outputs are communicated to the external device or communication network, and analysis of the detector outputs is performed at the external device (e.g., personal electronic device $60) or communication network.

In some embodiments of any of the aspects listed above, the device also includes a casing (e.g., casing $01) for housing the chassis, the control system, and/or the power source, wherein the first side of the chassis faces an exterior of the casing. In some embodiments, the casing is configured to be fitted into different enclosures, e.g., a handheld enclosure (e.g., handheld enclosure $50) or a wearable enclosure (e.g., wearable enclosure $70). In some embodiments, the casing is sleevable between the ROI and a wrapper (e.g., wearable enclosure $70), and able to be snapped-fitted into a housing comprising a gripping knob (e.g., handheld enclosure $50).

In some embodiments of any of the aspects listed above, the device also includes an internal casing display, mounted casing display, or external display.

In one aspect, the disclosure provides a device having a chassis e.g., chassis $20), a light source (e.g., light source $02-a, which is part of illumination assembly $02), a lens (e.g., lens assembly $06), an optical filter (e.g., filter $08-a, which is part of filter unit $08), an optical detector (e.g., detector $10-a, which is part of photo-sensor unit $10), a control system, and a power source (e.g., battery $18-a, which is part of power unit $18).

In some embodiments, the light source (e.g., light source $02-a) is attached to or disposed at the chassis (e.g., chassis $20) and configured to illuminate a region of interest (ROI). In some embodiments, the device is configured to illuminate and image a ROI (e.g., ROI 103 in FIG. 1) of less than 700 mm², less than 650 mm², less than 600 mm², less than 550 mm², less than 500 mm², less than 450 mm², less than 400 mm², less than 350 mm², less than 300 mm², less than 250 mm², less than 200 mm², less than 150 mm², less than 100 mm², or less than 50 mm² . . . . In some embodiments, the light source includes a plurality of light source elements.

In some embodiments, the lens (e.g., lens assembly $06) is attached to or disposed within the chassis (e.g., chassis $20) and configured to collect light backscattered by the ROI. In some embodiments, the lens includes a plurality of lens elements. For example, in some embodiments, lens assembly 506 includes an array of lens elements (e.g., an array of lens elements 706-c-n), each of which is in optical communication with a different color filter element in optical filter elements 708-a-n, as illustrated in FIG. 7A.

In some embodiments, the optical filter (e.g., filter $08-a, which is part of filter unit $08) is attached to or disposed within the chassis (e.g., chassis $20) and configured to filter the light collected by the lens (e.g., lens assembly $06), where the optical filter includes a plurality of optical filter elements, where each respective optical filter element in the plurality of optical filter elements is in optical communication with the lens, allowing light of a corresponding spectral range in a plurality of spectral ranges to pass while blocking light of any other spectral range in the plurality of spectral ranges.

In some embodiments, the optical detector (e.g., detector $10-a, which is part of photo-sensor unit $10) is attached to or disposed within the chassis (e.g., chassis $20) and configured to resolve light filtered by the optical filter (e.g., filter $08-a), where the optical detector includes a plurality of optical detector elements. Each respective optical detector element in the plurality of optical detector elements is covered by a corresponding optical filter in the plurality of optical filters, e.g., thereby configuring the device to generate a plurality of detector outputs.

In some embodiments, the control system controls operation of the light source and the optical detector.

In some embodiments, the power source (e.g., battery $18-a, which is part of power unit $18) is in electrical communication with the light source, the optical detector, and the control system. In some embodiments, the power source has a nominal voltage of 10 volts or less, e.g., no more than 10, 9, 8, 7, 6, 5, 4, 3, or fewer volts, and is configured to provide electrical power for operating the light source, the optical detector, and the control system.

In some embodiments, the control system includes one or more processors, memory, and one or more programs stored in the memory and configured to be executed by the one or more processors. The one or more programs include instructions for turning on the light source to illuminate the ROI, and using the optical detector to collect light, thereby generating a plurality of detector outputs. In some embodiments, the one or more programs stored in the memory further include instructions for performing a spectral analysis on the plurality of detector outputs to determine a concentration value of each respective spectral signature in one or more spectral signatures associated with the ROI. In other embodiments, spectral analysis is performed on a separate device, e.g., a personal electronic device or within a cloud server.

In some embodiments, the optical detector includes one or more photodiodes, a charge-coupled device (CCD), a complementary metal-oxide-semiconductor (CMOS), an active-pixel sensor (APS), or any combination thereof. In some embodiments, each respective optical detector element in the plurality of optical detector elements includes a single photodiode. In some embodiments, each respective optical detector element in the plurality of optical detector elements includes a single pixel or a cluster of pixels of the CCD, CMOS or APS.

In some embodiments, the disclosure provides a device having a chassis e.g., chassis $20), a light source (e.g., light source $02-a, which is part of illumination assembly $02), a lens (e.g., lens assembly $06), an optical filter (e.g., filter $08-a, which is part of filter unit $08), an optical detector (e.g., detector $10-a, which is part of photo-sensor unit $10), a control system (e.g., control system 114), and a power source (e.g., battery $18-a, which is part of power unit $18).

In some embodiments, the light source (e.g., light source $02-a) is attached to or disposed at the chassis (e.g., chassis $20) and configured to illuminate a region of interest (ROI). In some embodiments, the device is configured to illuminate and image a ROI of less than 700 mm², less than 650 mm², less than 600 mm², less than 550 mm², less than 500 mm², less than 450 mm², less than 400 mm², less than 350 mm², less than 300 mm², less than 250 mm², less than 200 mm², less than 150 mm², less than 100 mm², or less than 50 mm² . . . . In some embodiments, the light source includes a plurality of light source elements.

In some embodiments, the lens (e.g., lens assembly $06) is attached to or disposed within the chassis (e.g., chassis $20) and configured to collect light backscattered by the ROI. In some embodiments, the lens includes a plurality of lens elements. For example, in some embodiments, lens assembly 706 includes an array of lens elements 706-c-n, each of which is in optical communication with a different color filter element 708-a-n, as illustrated in FIG. 7A.

In some embodiments, the optical filter (e.g., filter $08-a, which is part of filter unit $08) is attached to or disposed within the chassis (e.g., chassis $20) and configured to filter the light collected by the lens (e.g., lens assembly $06). The optical filter includes an array of filter regions, each filter region in the array of filter regions comprises a plurality of optical filter elements. Filter arrays for hyperspectral imaging are described, for example, in U.S. Pat. No. 9,766,382, the disclosure of which is expressly incorporated herein by reference, in its entirety, for all purposes. Each respective optical filter element in the plurality of optical filter elements allows light, e.g., backscattered by the ROI, of a corresponding spectral range in a plurality of spectral ranges to pass while blocking light of any other spectral range in the plurality of spectral ranges.

In some embodiments, the optical detector (e.g., detector $10-a, which is part of photo-sensor unit $10) is attached to or disposed within the chassis (e.g., chassis $20) and configured to resolve light filtered by the optical filter (e.g., filter $08-a). The optical detector includes an array of detector regions (e.g., regions 708-a-n in FIG. 7A). Each detector region in the array of detector regions includes a plurality of optical detector elements, where each respective optical detector element in the plurality of optical detector elements is covered by a corresponding optical filter element in the plurality of optical filter elements, e.g., thereby capable of generating a hyperspectral data cube of detector outputs from a single image capture.

In some embodiments, the control system controls operation of the light source and the optical detector.

In some embodiments, the power source (e.g., battery $18-a, which is part of power unit $18) is in electrical communication with the light source, the optical detector, and the control system. In some embodiments, the power source has a nominal voltage of 10 volts or less, e.g., no more than 10, 9, 8, 7, 6, 5, 4, 3, or fewer volts, and is configured to provide electrical power for operating the light source, the optical detector, and the control system.

In some embodiments, the control system includes one or more processors, memory, and one or more programs stored in the memory and configured to be executed by the one or more processors. The one or more programs include instructions for turning on the light source to illuminate the ROI, and using the optical detector to collect light, thereby generating a plurality of detector outputs for construction of a hyperspectral data cube. In some embodiments, the one or more programs stored in the memory further include instructions for performing a spectral analysis on the plurality of detector outputs to determine a concentration value of each respective spectral signature in one or more spectral signatures associated with the ROI.

In some embodiments, the optical detector includes one or more photodiodes, a charge-coupled device (CCD), a complementary metal-oxide-semiconductor (CMOS), an active-pixel sensor (APS), or any combination thereof. In some embodiments, each respective optical detector element in the plurality of optical detector elements includes a single photodiode. In some embodiments, each respective optical detector element in the plurality of optical detector elements includes a single pixel or a cluster of pixels of the CCD, CMOS or APS.

In one aspect, the disclosure provides a device having a chassis e.g., chassis $20), a light source (e.g., light source $02-a, which is part of illumination assembly $02), a lens (e.g., lens assembly $06), an optical filter (e.g., optical filters $02-c, which are part of illumination assembly $02), an optical detector (e.g., detector $10-a, which is part of photo-sensor unit $10), a control system, and a power source (e.g., battery $18-a, which is part of power unit $18).

In some embodiments, the light source (e.g., light source $02-a) is attached to or disposed at the chassis (e.g., chassis $20) and configured to illuminate a region of interest (ROI). In some embodiments, the device is configured to illuminate and image a ROI of less than 700 mm², less than 650 mm², less than 600 mm², less than 550 mm², less than 500 mm², less than 450 mm², less than 400 mm², less than 350 mm², less than 300 mm², less than 250 mm², less than 200 mm², less than 150 mm², less than 100 mm², or less than 50 mm² . . . . In some embodiments, the light source includes a plurality of light source elements.

In some embodiments, the optical filter (e.g., optical filter $02-c, which is part of illumination assembly $02) include a plurality of optical filter elements (e.g., filter elements $02-c-n), where each respective optical filter element in the plurality of optical filter elements covers a corresponding light source element in the plurality of light source elements (e.g., light source $02-a), and allows light of a corresponding spectral range in a plurality of spectral ranges to pass while blocking light of any other spectral range in the plurality of spectral ranges.

In some embodiments, the lens (e.g., lens assembly $06) is attached to or disposed within the chassis (e.g., chassis $20), and configured to collect light backscattered by the ROI. In some embodiments, the lens includes a plurality of lens elements (e.g., lens elements 706-c-n). For example, in some embodiments, lens assembly 506 includes an array of lens elements, each of which is in optical communication with a different optical detector element 510-a-n, as illustrated in FIG. 5A. FIG. 6A includes an illustration of optical filter 708-a positioned between lens assembly 606 and optical detector elements 610-a-n. FIG. 7A includes an illustration of a lens 706-a (e.g., a lens array) positioned between optical filter 708-a. FIG. 7A further illustrates polarizer 706-b positioned adjacent to optical filter 708-a facing an exterior of imaging device 700.

In some embodiments, the optical detector (e.g., detector $10-a, which is part of photo-sensor unit $10) is attached to or disposed within the chassis (e.g., chassis $20), in optical communication with the lens (e.g., lens assembly $06), and configured to resolve the light collected by the lens. The optical detector includes an array of optical detector elements (e.g., regions of optical detector 710 or regions of a two-dimensional detector array). In some embodiments, this configuration is capable of generating an array of detector outputs for each pair of the light source element and the filter element.

In some embodiments, the device includes a control system to control the operation of the light source and the optical detector and a power source in electrical communication with the light source, the optical detector and the control system. The power source has a nominal voltage of 10 volts or less (e.g., no more than 10, 9, 8, 7, 6, 5, 4, 3, or fewer volts) and is configured to provide electrical power for operating the light source, the optical detector and the control system.

In some embodiments, the control system controls operation of the light source and the optical detector.

In some embodiments, the power source (e.g., battery $18-a, which is part of power unit $18) is in electrical communication with the light source, the optical detector, and the control system. In some embodiments, the power source has a nominal voltage of 10 volts or less, e.g., no more than 10, 9, 8, 7, 6, 5, 4, 3, or fewer volts, and is configured to provide electrical power for operating the light source, the optical detector, and the control system.

In some embodiments, the control system includes one or more processors, memory, and one or more programs stored in the memory and configured to be executed by the one or more processors. The one or more programs include instructions for sequentially turning on and off each respective light source element in the plurality of light source elements to illuminate the ROI sequentially, and sequentially using the optical detector to collect light when each respective light source element is turned on, thereby generating a plurality of arrays of detector outputs, each array of detector outputs at a corresponding spectral range in the plurality of spectral ranges, where the plurality of arrays of detector outputs collectively forms the hyperspectral data cube of detector outputs. In some embodiments, the one or more programs stored in the memory further include instructions for performing a spectral analysis on the plurality of detector outputs to determine a concentration value of each respective spectral signature in one or more spectral signatures associated with the ROI.

In some embodiments, the optical detector includes one or more photodiodes, a charge-coupled device (CCD), a complementary metal-oxide-semiconductor (CMOS), an active-pixel sensor (APS), or any combination thereof. In some embodiments, each respective optical detector element in the plurality of optical detector elements includes a single photodiode. In some embodiments, each respective optical detector element in the plurality of optical detector elements includes a single pixel or a cluster of pixels of the CCD, CMOS or APS.

In some embodiments of any of the aspects listed above, a spectral signature in the one or more spectral signatures is oxyhemoglobin, deoxyhemoglobin, melanin, or a combination thereof. In some embodiments, an oxygen saturation value or an oximetry index value is determined based on the concentration values of the one or more spectral signatures.

In some embodiments of any of the aspects listed above, the inner perimeter wall forms a, e.g., cylindrical, structure having a nominal diameter that is between 5 mm and 10 mm, between 10 and 15 mm, or between 15 and 20 mm. In some embodiments, the structure has a circular, oval, or polygonal cross-section. In some embodiments, the height of the inner perimeter wall along the axis of the chassis is less than 25 mm, less than 20 mm, less than 15 mm, less than 10 mm, less than 8 mm, less than 6 mm, or less than 4 mm.

In some embodiments of any of the aspects listed above, the acute angle at which the oblique outer wall is extended with respect to the axis of the chassis is between 15 degrees and 30 degrees, between 30 degrees and 45 degrees, between 45 degrees and 60 degrees, or between 60 degrees and 75 degrees.

In some embodiments of any of the aspects listed above, the oblique outer wall forms a truncated conical structure, where a nominal diameter of the truncated conical structure at the second side of the chassis is between 10 mm and 15 mm, between 15 mm and 20 mm, between 20 mm and 25 mm, or between 25 mm and 30 mm, and a nominal diameter of the truncated conical structure at the first side of the chassis is between 15 mm and 25 mm, between 25 mm and 35 mm, or between 35 and 45 mm. In some embodiments, the truncated conical structure has a circular, oval, or polygonal cross-section.

In some embodiments of any of the aspects listed above, the oblique outer wall has a height along the axis of the chassis that is less than 14 mm, less than 12 mm, less than 10 mm, less than 8 mm, or less than 6 mm.

In some embodiments of any of the aspects listed above, the light source comprises three, four, five, six, seven, eight, or more than eight light source elements. In some embodiments, the three, four, five, six, seven, eight or more than eight light source elements are uniformly distributed around the axis of the chassis at the oblique outer wall of the chassis.

In some embodiments of any of the aspects listed above, the light source emits near infrared light, visible light, ultraviolet light, or any combination thereof.

In some embodiments of any of the aspects listed above, each light source element of the light source emits white light having a spectrum between 400 nm and 780 nm.

In some embodiments of any of the aspects listed above, the ROI has a size less than 700 mm², less than 650 mm², less than 600 mm², less than 550 mm², less than 500 mm², less than 450 mm², less than 400 mm², less than 350 mm², less than 300 mm², less than 250 mm², less than 200 mm², less than 150 mm², less than 100 mm², or less than 50 mm² . . .

In some embodiments of any of the aspects listed above, the ROI is located at a range that is less than 50 mm, less than 40 mm, less than 30 mm, less than 25 mm, less than 20 mm, 18 mm, less than 16 mm, less than 14 mm, less than 12 mm, less than 10 mm, less than 8 mm, less than 6 mm, less than 4 mm, or less than 3 mm with respect to the second side of the chassis.

In some embodiments of any of the aspects listed above, each respective optical filter element in the plurality of optical filter elements is a bandpass filter. In some embodiments, each respective optical filter element in the plurality of optical filter elements has a different spectral band than any other optical filter element in the plurality of optical filter elements. In some embodiments, at least two optical filter elements in the plurality of optical filter elements have a same spectral band.

In some embodiments of any of the aspects listed above, the device also includes a first polarizer comprising one or more first polarizer elements (e.g., filter element $02-c), where the first polarizer is disposed in an optical path between the light source (e.g., light source $02-a) and the ROI, and configured to selectively allow light that is substantially limited to a first polarization to pass through. In some such embodiments, the device also includes a second polarizer, e.g., including one or more polarizer elements (e.g., filter element $08-b), where the second polarizer is disposed in an optical path between the ROI and the optical detector (e.g., optical detector $10-a), and configured to selectively allow light that is substantially limited to a second polarization to pass through, where the second polarization is in a different direction from the first polarization.

In some embodiments, a battery in the one or more batteries includes a lithium button cell and/or a lithium polymer battery, e.g., having a voltage of less than 10 volts, less than 5 volts, or less than 3 volts.

In some embodiments of any of the aspects listed above, the control system also includes a power regulator (e.g., power regulator $18-b, which is part of power unit $18) that maintains a power supply at a desired level.

In some embodiments of any of the aspects listed above, the control system also includes a communication interface (e.g., communication interface $16, e.g., including a communication element $16-a and/or communication antenna $16-b) for wired or wireless communication with an external device or communication network. In some embodiments, the detector outputs are communicated to the external device or communication network, and analysis of the detector outputs is performed at the external device (e.g., personal electronic device $60) or communication network.

In some embodiments of any of the aspects listed above, the device also includes a casing (e.g., casing $01) for housing the chassis, the control system, and/or the power source, wherein the first side of the chassis faces an exterior of the casing. In some embodiments, the casing is configured to be fitted into different enclosures, e.g., a handheld enclosure (e.g., handheld enclosure $50) or a wearable enclosure (e.g., wearable enclosure $70). In some embodiments, the casing is sleevable between the ROI and a wrapper (e.g., wearable enclosure $70), and able to be snapped-fitted into a housing comprising a gripping knob (e.g., handheld enclosure $50).

In some embodiments of any of the aspects listed above, the device also includes an internal casing display, mounted casing display, or external display.

In some embodiments of any of the aspects listed above the control system includes a mobile application (e.g., an application on a personal electronic device $70, for controlling the imaging device on the personal electronic device).

In another aspect, a system (e.g., distributed client-server system 900) includes the device (e.g., imaging device 904 corresponding to imaging devices $00 described with respect to FIGS. 5A-7C), a first client device (e.g., client system 902-A) in wireless communication with the device, and a server (e.g., server system 906-A) in wireless communication with the device and the first client device. The server includes one or more central processing units, memory, and one or more programs. The one or more programs are stored in the memory and are configured to be executed by the one or more central processing units. The one or more programs including instructions for receiving, from the device, the hyperspectral data cube of detector outputs, forming a hyperspectral image using the hyperspectral data cube of detector outputs, and transmitting, to the first client device, the hyperspectral image.

In some embodiments, forming the hyperspectral image includes determining the oxygen saturation value or the oximetry index value based on the concentration values of the one or more spectral signatures in the hyperspectral image and transmitting the hyperspectral image to the first client device includes transmitting the oxygen saturation value or the oximetry index value.

In some embodiments, the first client device is a mobile device (e.g., client system 902-A of FIG. 9 corresponding to user device 460 in FIG. 4) operated by a patient, the system thereby enabling the patient to self-monitor a condition associated with the oxygen saturation value or the oximetry index value.

### General Considerations

In various embodiments, the present invention provides hyperspectral imaging systems, methods and devices for determining hyperspectral signature(s) of a region of interest (ROI), e.g., on a human subject. The structure of skin, while complex, can be approximated as two separate and structurally different layers, namely the epidermis and dermis. Below the dermis is a closely associated layer, namely the deeper subcutaneous tissue (hypodermis). The epidermis, dermis, and deeper subcutaneous tissue have very different scattering and absorption properties due to differences of composition.

The epidermis is the outer layer of skin. It has specialized cells called melanocytes that produce melanin pigments. Melanin is the major chromophore in the epidermis in the visible range. Its absorption profile demonstrates an exponential-like decay towards the red part of the spectrum. For further details, see G. H. Findlay, “Blue Skin,” British Journal of Dermatology 83 (1), 127-134 (1970), the content of which is incorporated herein by reference in its entirety for all purposes.

The dermis has a dense collection of collagen fibers and blood vessels, and its optical properties are very different from that of the epidermis. Absorption of light of a bloodless dermis is negligible. However, blood-born pigments like oxy- and deoxy-hemoglobin and water are major absorbers of light in the dermis. Scattering by the collagen fibers and absorption due to chromophores in the dermis determine the depth of penetration of light through skin.

The deeper subcutaneous tissue (hypodermis) is made of fat and connective tissue. It contains larger blood vessels (e.g., veins, arteries) and nerves than those found in the dermis. Blood-born pigments like oxy- and deoxy-hemoglobin and water are major absorbers of light in the deeper subcutaneous tissue.

Light used to illuminate the surface of a subject will penetrate into the skin. The extent to which the light penetrates depends on the wavelength of the particular radiation. For example, with respect to visible light, the longer the wavelength, the farther the light will penetrate into the skin. For example, only about 32% of 400 nm violet light penetrates into the dermis of human skin, while greater than 85% of 700 nm red light penetrates into the dermis or beyond (see, Capinera J. L., Encyclopedia of Entomology, 2nd Edition, Springer Science (2008) at page 2854, the content of which is hereby incorporated herein by reference in its entirety for all purposes).

Non-limiting examples of conditions that can be evaluated by the methods and the devices of the present invention include: tissue ischemia, ulcer formation, ulcer progression, pressure ulcer formation, pressure ulcer progression, diabetic foot ulcer formation, diabetic foot ulcer progression, venous stasis, venous ulcer disease, infection, shock, cardiac decompensation, respiratory insufficiency, hypovolemia, the progression of diabetes, congestive heart failure, sepsis, dehydration, hemorrhage, hypertension, exposure to a chemical, exposure to a biological agent, exposure to radiation (including but not limited to radiation therapy dosages), an inflammatory response, wound healing prediction, and wound formation prediction. Greater details on examples of conditions that can be evaluated by the method are provided elsewhere herein.

A bandpass filter of a filter element can be described in terms of its “characteristic wavelength,” e.g., the wavelength at which the filter is most transparent, or its “center wavelength,” e.g., the component wavelength at the midpoint of the spectral bandpass. In certain implementations, the bandpass filter is characterized by both its characteristic or center wavelength and its spectral bandwidth. For example, a bandpass filter with a center wavelength of 340±2 nm, a full-width half maximum (FWHM) bandwidth of 10±2, and a peak transmission (e.g., the maximum percentage transmission within the bandpass) of 50%, allows at least 25% of each component light having a wavelength from 330±4 nm to 350±4 nm to pass through.

In specific implementations, a filter element is a bandpass filter, e.g., a filter that allows only radiation having a wavelength in a certain range to pass, while blocking passage of other wavelengths. In certain embodiments, the FWHM spectral bandpass of a filter element (e.g., the size of the bandpass transmitted through the filter) is no more than about 100 nm, preferably no more than about 50 nm, more preferably no more than about 25 nm. In yet other embodiments, the FWHM spectral bandwidth of a filter element 211 is no more than 250 nm, 200 nm, 200 nm, 175 nm, 150 nm, 150 nm, 125 nm, 100 nm, 90 nm, 80 nm, 75 nm, 70 nm, 65 nm, 60 nm, 55 nm, 50 nm, 45 nm, 40 nm, 35 nm, 30 nm, 25 nm, 20 nm, 15 nm, 10 nm, 9 nm, 8 nm, 7 nm, 6 nm, 5 nm, 4 nm, 3 nm, 2 nm, or 1 nm.

In certain implementations, the bandpass filter of a filter element is a narrow pass filter. In specific implementations, the narrow pass filter has a FWHM spectral bandwidth of no more than 25 nm, 24 nm, 23 nm, 22 nm, 21 nm, 20 nm, 19 nm, 18 nm, 17 nm, 16 nm, 15 nm, 14 nm, 13 nm, 12 nm, 11 nm, 10 nm, 9 nm, 8 nm, 7 nm, 6 nm, 5 nm, 4 nm, 3 nm, 2 nm, or 1 nm.

In some implementations, the filter elements are a plurality of bandpass filters having central wavelengths that are separated by at least 10 nm, or at least 15 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, or more.

### Application for Home Healthcare/Telemedicine.

In some implementations, the systems and methods of the present disclosure are implemented for a telemedicine setting. As used herein, telemedicine refers to a practice of monitoring and treating a patient remotely so that a physician/healthcare provider (e.g., a physician or healthcare provider located at a hospital or a clinic) and the patient are not physically present for each other. In some implementations, the telemedicine setting allows a patient to do the measurement at the patient's home while the physician communicates with the patient remotely. For example, the physician is located at a hospital or a clinic. In some implementations, the telemedicine setting allows a patient to have a measurement done in a remote facility (e.g., a pharmacy or a nursing home) while the physician is located at the hospital or clinic.

In some implementations, distributed client-server system 900 including a plurality of client systems 902 is applied for telemedicine. For example, a patient performs a measurement using an imaging device $00, as described with respect to FIGS. 3 and 4. In some embodiments, the patient performs the measurement at his or her home. The imaging data is then transferred to client system 902-A. In some implementations, client system 902-A is operated by the patient. The imaging data is processed and analyzed as described with respect to FIG. 9 using distributed client-server system 900. In some implementation, the processed hyperspectral images (e.g., processed hyperspectral image 905) are returned to client system 902-A for the patient's review. Alternatively, the processed hyperspectral images are transferred to another client system (e.g., client system 902-N) to be reviewed by the patient's physician or healthcare provider. For example, client system 902-N is operated by a hospital or a clinic. Alternatively, client system 902-A is operated by a remote medical facility (e.g., a nursing home or a pharmacy) and the client system 902-N is operated by the hospital or a clinic.

In general, the methods, systems and devices of the present disclosure provide for an efficient, flexible, and convenient approach for diagnosis and treatment of patients in a telemedicine setting. The present disclosure allows a patient to be treated at a location convenient for him or her (e.g., at home or at a nearby pharmacy) without the need for visiting a hospital. The present disclosure also allows for a physician to be able to treat patients efficiently in a variety of locations simultaneously.

In one aspect, the present disclosure provides a method for remote monitoring a condition associated with changes in tissue oximetry. The method includes collecting a plurality of hyperspectral data sets of a region of interest on a subject over time (e.g., a series of images or spot measurements collected at a plurality of wavebands, as described herein), using a portable hyperspectral imaging system as described herein, e.g., every 8 hours, every 12 hours, daily, every other day, weekly, monthly, etc. After collection of each respective hyperspectral data set, the imaging system transmits the respective image set to an external processing device, which determines one or more values for the tissue oxyhemoglobin or deoxyhemoglobin of the region of interest on the subject.

In some embodiments, the hyperspectral data sets are transmitted to a personal electronic device (e.g., smart phone, tablet, laptop, or desktop computer) of the subject, which either processes the data set itself (e.g., in simple implementations, for example, where the imaging system collects spot measurements, rather than 2-dimensional images, at each waveband), or transmits the data set to an external server for processing (e.g., in the cloud). In some embodiments, where the personal electronic device transmits the data set to an external server for processing, the person electronic device receives results from the external server, e.g., an indication of the patient's condition and/or a pseudo-color hyperspectral image of the region of interest. In some embodiments, the personal electronic device then provides the subject with advice on the results of the analysis, e.g., that the patient is fine or that the patient should seek medical attention. In some embodiments, the personal electronic device and/or the remote server transmits results of the analysis to a medical professional, who can then evaluate the condition of the subject without having to personally interface with the subject. In some embodiments, the personal electronic device and/or the remote server only transmits the results of the hyperspectral data analysis when the subject may require medical attention. In other embodiments, the personal electronic device and/or the remote server sends a summary of the subject's data over a time period, e.g., weekly, every two weeks, or monthly. In some embodiments, a histogram, or other summary table, chart, or graph, of the patient's tissue perfusion results are transmitted to the medical professional for evaluation.

Additional, Optional, and/or Alternative Components.

In some implementations, the light sources of the imaging devices described herein generates light having spectrum that includes a plurality of component wavelengths. The spectrum can include component wavelengths in the X-ray band (in the range of about 0.01 nm to about 10 nm); ultraviolet (UV) band (in the range of about 10 nm to about 400 nm); visible band (in the range of about 400 nm to about 700 nm); near infrared (NIR) band (in the range of about 700 nm to about 2500 nm); mid-wave infrared (MWIR) band (in the range of about 2500 nm to about 10 μm); long-wave infrared (LWIR) band (in the range of about 10 μm to about 100 μm); terahertz (THz) band (in the range of about 100 μm to about 1 mm); or millimeter-wave band (also referred to as the microwave band) in the range of about 1 mm to about 300 mm, among others. The NIR, MWIR, and LWIR are collectively referred to herein as the infrared (IR) band. The light can include a plurality of component wavelengths within one of the bands, e.g., a plurality of wavelengths in the NIR band, or in the THz. Alternately, the light can include one or more component wavelengths in one band, and one or more component wavelengths in a different band, e.g., some wavelengths in the visible, and some wavelengths in the IR. Light with wavelengths in both the visible and NIR bands is referred to herein as “VNIR.” Other useful ranges may include the region 1,000-2,500 nm (shortwave infrared, or SWIR). Examples of illumination configurations are disclosed in WO 2014/063117, US 2013/0137961, US 2015/0271380, US 2016/0249810, US 2017/0067781, US 2017/0150903, and US 2017/0272666, each of which is hereby incorporated by reference herein in its entirety.

In some implementations, an illumination assembly $02 includes one or more light sources. For example, an illumination assembly can include a single broadband light source, a single narrowband light source, a plurality of narrowband light sources, or a combination of one or more broadband light source and one or more narrowband light source. By “broadband” it is meant light that includes component wavelengths over a substantial portion of at least one band, e.g., over at least 20%, or at least 30%, or at least 40%, or at least 50%, or at least 60%, or at least 70%, or at least 80%, or at least 90%, or at least 95% of the band, or even the entire band, and optionally includes component wavelengths within one or more other bands. A “white light source” is considered to be broadband, because it extends over a substantial portion of at least the visible band. By “narrowband” it is meant light that includes components over only a narrow spectral region, e.g., less than 20%, or less than 15%, or less than 10%, or less than 5%, or less than 2%, or less than 1%, or less than 0.5% of a single band. Narrowband light sources need not be confined to a single band, but can include wavelengths in multiple bands. A plurality of narrowband light sources may each individually generate light within only a small portion of a single band, but together may generate light that covers a substantial portion of one or more bands, e.g., may together constitute a broadband light source.

One example of a suitable light source is a diffused lighting source that uses a halogen lamp, such as the Lowel Pro-Light Focus Flood Light. A halogen lamp produces an intense broad-band white light which is a close replication of daylight spectrum. Other suitable light sources include a xenon lamp, a hydrargyrum medium-arc iodide lamp, and/or a light-emitting diode. In some embodiments, a light source is tunable. Other types of light sources are also suitable.

In an implementation, the illumination assembly comprises one or more incandescent lights, one or more xenon lamps, one or more halogen lamps, one or more hydrargyrum medium-arc iodide, and one or more broadband light emitting diodes (LEDs), or any combination thereof. In another implementation, the illumination assembly comprises a first light source emitting light that is substantially limited to the first spectral range and a second light source emitting light that is substantially limited to the second spectral range.

In some implementations, the illumination assembly comprises a plurality of light sources radially disposed on the chassis $20. In an implementation, the illumination assembly includes a plurality of light source sets radially disposed on the chassis. Each light source set in the plurality of light source sets comprises a first light source that emits light that is substantially limited to the first spectral range and a second light source that emits light that is substantially limited to the second spectral range. Each light source in each light source set in the plurality of light source sets is offset from the lens assembly and positioned so that light from each respective light source is backscattered by the ROI of the subject and then passed through the lens assembly. Each light in each light source set has a different radial position with respect to the lens assembly. Examples of light source arrangement are disclosed in US 2016/0249810 and U.S. Pat. No. 9,526,427, each of which is hereby incorporated by reference herein in its entirety.

Depending on the particular light source used, the relative intensities of the light's component wavelengths are uniform (e.g., are substantially the same across the spectrum), or vary smoothly as a function of wavelength, or are irregular (e.g., in which some wavelengths have significantly higher intensities than slightly longer or shorter wavelengths), and/or can have gaps. Alternatively, the light can include one or more narrow-band spectra in regions of the electromagnetic spectrum that do not overlap with each other.

In some implementations, an illumination assembly $02 includes a lens to modify the focal properties of the light emitted from one or more light sources. In an implementation, the lens is selected such that an ROI of a subject can be substantially uniformly irradiated. That is, the intensity of light at one sub-region of the ROI is substantially the same as the intensity of light at another sub-region of the ROI. In another implementation, the intensity of the light varies from one sub-region to another.

In some implementations, the imaging device includes one or more first polarizers disposed in front of the illumination assembly and configured to remove any light that does not have a selected polarization. The one or more polarizers can be, for example, a polarizing beam splitter or a thin film polarizer. The polarization can be selected, for example, by rotating the one or more polarizers appropriately.

In some implementations, the imaging device also includes one or more second polarizers configured to remove any light that does not have a selected polarization. The one or more polarizers can be, for example, a polarizing beam splitter or a thin film polarizer. The polarization can be selected, for example, by rotating the one or more polarizers appropriately.

In some implementations, the one or more first polarizer is configured to selectively allow light that is substantially limited to at least one first polarization to pass through; and the one or more second polarizer is configured to selectively allow light that is substantially limited to at least one second polarization to pass through. Each polarization in the at least one first polarization is in a different direction from the at least one second polarization, and each polarization in the at least one second polarization is in a different direction from the at least one first polarization. In an implementation, a polarization in the at least one first polarization is substantially perpendicular to a polarization in the at least second polarization.

In some implementations, the imaging device includes an imaging unit $04, an illumination assembly $02, one or more central processing units (CPUs) $14, an optional main non-volatile storage unit, an optional controller, a system memory for storing system control programs, data, and/or application programs, including programs and data optionally loaded from a non-volatile storage unit. In some implementations the non-volatile storage unit includes a memory card, for storing software and data. The storage unit is optionally controlled by the controller.

In some implementations, an imaging device optionally includes a user interface including one or more input devices (e.g., a touch screen, buttons, or switches) and/or an optional display. Additionally and/or alternatively, in some implementations, the imaging device may be controlled by an external device such as a handheld device, a smartphone (or the like), a tablet computer, a laptop computer, a desktop computer, and/or a server system. To that end, the imaging device includes one or more communication interfaces $16 for connecting to any wired or wireless external device or communication network (e.g., a wide area network such as the Internet). The imaging device includes an internal bus for interconnecting aforementioned elements. The communication bus may include circuitry (sometimes called a chipset) that interconnects and controls communications between the aforementioned components.

In some implementations, the imaging device communicates with a communication network, thereby enabling the imaging device to transmit and/or receive data between mobile communication devices over the communication network, particularly one involving a wireless link, such as cellular, WiFi, ZigBee, BlueTooth, IEEE 802.11b, 802.11a, 802.11g, or 802.11n, etc. The communication network can be any suitable communication network configured to support data transmissions. Suitable communication networks include, but are not limited to, cellular networks, wide area networks (WANs), local area networks (LANs), the Internet, IEEE 802.11b, 802.11a, 802.11g, or 802.11n wireless networks, landline, cable line, fiber-optic line, etc. The imaging system, depending on an embodiment or desired functionality, can work completely offline by virtue of its own computing power, on a network by sending raw or partially processed data, or both concurrently.

In some implementations, the imaging device does not include a controller or a storage unit. In some such implementations, the memory and CPU are one or more application-specific integrated circuit chips (ASICs) and/or programmable logic devices (e.g. an FGPA-Field Programmable Gate Array). For example, in some implementations, an ASIC and/or programmed FPGA includes the instructions of the illumination control module, optical control module, data processing module and/or communication interface control module. In some implementations, the ASIC and/or FPGA further includes storage space for the data store, the sensor data stored therein and/or the hyperspectral data cubes stored therein.

In some implementations, the system memory or an external device stores a spectral library and spectral analyzer for comparing hyperspectral data generated by the image device to known spectral patterns associated with various medical conditions. In some implementations, analysis of the acquired hyperspectral data is performed on an external device such as a handheld device, tablet computer, laptop computer, desktop computer, an external server, for example in a cloud computing environment.

In some implementations, a spectral library includes profiles for a plurality of medical conditions, each of which contain a set of spectral characteristics unique to the medical condition. A spectral analyzer uses the spectral characteristics to determine the probability that a region of the subject corresponding to a measured hyperspectral data cube is afflicted with the medical condition. In some implementations, each profile includes additional information about the condition, e.g., information about whether the condition is malignant or benign, options for treatment, etc. In some implementations, each profile includes biological information, e.g., information that is used to modify the detection conditions for subjects of different skin types. In some implementations, the spectral library is stored in a single database. In other implementations, such data is instead stored in a plurality of databases that may or may not all be hosted by the same computer, e.g., on two or more computers addressable by wide area network. In some implementations, the spectral library is electronically stored in the storage unit and recalled using the controller when needed during analysis of hyperspectral data cube data.

In some implementations, the spectral analyzer analyzes a particular spectra derived from hyperspectral data cube data, the spectra having pre-defined spectral ranges (e.g., spectral ranges specific for a particular medical condition), by comparing the spectral characteristics of a pre-determined medical condition to the subject's spectra within the defined spectral ranges. In some implementations, the pre-defined spectral ranges correspond to values of one or more of deoxyhemoglobin levels, oxyhemoglobin levels, total hemoglobin levels, oxygen saturation, oxygen perfusion, hydration levels, total hematocrit levels, melanin levels, and collagen levels of a tissue on a patient (e.g., an area of the body of a subject). Performing such a comparison only within defined spectral ranges can both improve the accuracy of the characterization and reduce the computational power needed to perform such a characterization.

In some implementations, the medical condition is selected from the group consisting of tissue ischemia, ulcer formation, ulcer progression, pressure ulcer formation, pressure ulcer progression, diabetic foot ulcer formation, diabetic foot ulcer progression, venous stasis, venous ulcer disease, infection, shock, cardiac decompensation, respiratory insufficiency, hypovolemia, the progression of diabetes, congestive heart failure, sepsis, dehydration, hemorrhage, hypertension, exposure to a chemical, exposure to a biological agent, exposure to radiation (including but not limited to radiation therapy dosages), an inflammatory response, wound healing prediction, and wound formation prediction.

In some implementations, the medical condition is shock, such as hemorrhagic, hypovolemie, cardiogenic, neurogenie, septic, or burn shock. Shock refers to a life-threatening condition associated with circulatory failure leading to an inadequate delivery of oxygen and nutrients to vital organs. Shock may lead to cellular and tissue hypoxia. Information provided by hyperspectral imaging could support early detection of shock and provide information about likely outcomes. The hyperspectral imaging device of the present disclosure can be used to monitor oxygenation of a surface tissue (e.g., a surface tissue on the patient's arm or leg). As oxygenation of a surface tissue tends to reduce earlier than oxygenation of blood, monitoring the oxygenation of the surface tissue may provide an early indication of a development of the shock. The imaging device of the present disclosure may further be used to derive information regarding oxygen delivery, oxygen extraction, and hydration level that can be used to evaluate progress of the shock. Examples of detecting and monitoring shock using hyperspectral imaging devices are described in U.S. Patent Application Publication No. 2007/0024946, the content of which is expressly incorporated by reference herein, in its entirety, for all purposes. In some implementations, the spectral analyzer identifies a spectral signature within the hyperspectral data cube that corresponds with a medical condition of the patient. In certain implementations, this is accomplished by identifying a pattern of oxidation or hydration in a tissue associated with a tissue of the patient. In some implementations, the analysis of the hyperspectral data cube includes performing at least one of adjusting the brightness of at least one of the respective digital images in the hyperspectral data cube, adjusting the contrast of at least one of the respective digital images in the hyperspectral data cube, removing an artifact from at least one of the respective digital images in the hyperspectral data cube, processing one or more sub-pixels of at least one of the respective digital images in the hyperspectral data cube, and transforming a spectral hypercube assembled from a plurality of digital images.

In some implementations, a display receives an image (e.g., a color image, mono-wavelength image, or hyperspectral image) from a display control module, and displays the image. Optionally, the display subsystem also displays a legend that contains additional information. For example, the legend can display information indicating the probability that a region has a particular medical condition, a category of the condition, a probable age of the condition, the boundary of the condition, information about treatment of the condition, information indicating possible new areas of interest for examination, and/or information indicating possible new information that could be useful to obtain a diagnosis, e.g., another test or another spectral area that could be analyzed.

In some implementations, the communication interface includes a docking station for a mobile device having a mobile device display. A mobile device, such as a smart phone, a personal digital assistant (PDA), an enterprise digital assistant, a tablet computer, an IPOD, a digital camera, or a portable music player, can be connected to the docking station, effectively mounting the mobile device display onto the imaging device. Optionally, the mobile device is used to manipulate the displayed image and/or control the image device.

In some implementations, an imaging device disclosed herein is configured to be in wired or wireless communication with an external display, for example, on a handheld device, tablet computer, laptop computer, desktop computer, television, IPOD, or projector unit, on which the image is displayed. Optionally, a user interface on the external device is used to manipulate the displayed image and/or control the imaging device.

In some implementations, an image can be displayed in real time on the display. The real-time image can be used, for example, to focus an image of the subject, to select an appropriate region of interest, and to zoom the image of the subject in or out. In one embodiment, the real-time image of the subject is a color image captured by an optical detector that is not covered by a detector filter. In some implementations, the imager subsystem comprises an optical detector dedicated to capturing true color images of a subject. In some implementations, the real-time image of the subject is a monowavelength, or narrow-band (e.g., 10-50 nm), image captured by an optical detector covered by a detector filter. In these embodiments, any optical detector covered by a detector filter in the imager subsystem may be used for: (i) resolving digital images of the subject for integration into a hyperspectral data cube; and (ii) resolving narrow-band images for focusing, or otherwise manipulating the optical properties of the imaging device.

In some implementations, a hyperspectral image constructed from data collected by the imaging unit is displayed on an internal housing display, mounted housing display, or external display. Assembled hyperspectral data (e.g., present in a hyperspectral data cube) is used to create a two-dimensional representation of the imaged object or subject, based on one or more parameters. An image constructor module, stored in the imaging system memory or in an external device, constructs an image based on, for example, an analyzed spectrum. Specifically, the image constructor creates a representation of information within the spectra. In one example, the image constructor constructs a two-dimensional intensity map in which the spatially-varying intensity of one or more particular wavelengths (or wavelength ranges) within the spectra is represented by a corresponding spatially varying intensity of a visible marker.

In some implementations, the image constructor fuses a hyperspectral image with information obtained from one or more additional sensors. Non-limiting examples of suitable image fusion methods include: band overlay, high-pass filtering method, intensity hue-saturation, principle component analysis, and discrete wavelet transform.

The systems, methods and devices disclosed herein can be used to diagnose characterize a wide variety of medical conditions. They can be used locally or remotely. For instance, they can be used in distributed environments, clinic environments, and self/home diagnostic environments such as those disclosed in WO 2014/063117, which is hereby incorporated by reference herein in its entirety. The systems, methods and devices disclosed herein can provide more accurate and/or more thorough medical information about the ROI.

Non-limiting examples of conditions that can be evaluated by hyperspectral imaging, include: tissue ischemia, ulcer formation, ulcer progression, pressure ulcer formation, pressure ulcer progression, diabetic foot ulcer formation, diabetic foot ulcer progression, venous stasis, venous ulcer disease, infection, shock, cardiac decompensation, respiratory insufficiency, hypovolemia, the progression of diabetes, congestive heart failure, sepsis, dehydration, hemorrhage, hypertension, exposure to a chemical, exposure to a biological agent, exposure to radiation (including but not limited to radiation therapy dosages), an inflammatory response, wound healing prediction, and wound formation prediction.

In one embodiment, the concentration of one or more skin or blood component is determined in order to evaluate a medical condition in a patient. Non-limiting examples of components useful for medical evaluation include: deoxyhemoglobin levels, oxyhemoglobin levels, total hemoglobin levels, oxygen saturation, oxygen perfusion, hydration levels, total hematocrit levels, melanin levels, collagen levels, and bilirubin levels. Likewise, the pattern, gradient, or change over time of a skin or blood component can be used to provide information on the medical condition of the patient.

In one embodiment, the systems, methods and devices described herein are used to evaluate tissue oximetry and correspondingly, medical conditions relating to patient health derived from oxygen measurements in the superficial vasculature. In certain embodiments, the systems, methods and devices described herein allow for the measurement of oxygenated hemoglobin, deoxygenated hemoglobin, oxygen saturation, and oxygen perfusion. Processing of these data provide information to assist a physician with, for example, diagnosis, prognosis, assignment of treatment, assignment of surgery, and the execution of surgery for conditions such as critical limb ischemia, diabetic foot ulcers, pressure ulcers, peripheral vascular disease, surgical tissue health, etc.

In one embodiment, the systems, methods and devices described herein are used to evaluate diabetic and pressure ulcers. Development of a diabetic foot ulcer is commonly a result of a break in the barrier between the dermis of the skin and the subcutaneous fat that cushions the foot during ambulation. This rupture can lead to increased pressure on the dermis, resulting in tissue ischemia and eventual death, and ultimately manifesting in the form of an ulcer (Frykberg et al., Diabetes Care 1998; 21 (10): 1714-9). Measurement of oxyhemoglobin, deoxyhemoglobin, and/or oxygen saturation levels by hyperspectral imaging can provide medical information regarding, for example: a likelihood of ulcer formation at an ROI, diagnosis of an ulcer, identification of boundaries for an ulcer, progression or regression of ulcer formation, a prognosis for healing of an ulcer, the likelihood of amputation resulting from an ulcer. Further information on hyperspectral methods for the detection and characterization of ulcers, e.g., diabetic foot ulcers, are found in U.S. Patent Application Publication No. 2007/0038042, and Nouvong et al., Diabetes Care. 2009 November; 32 (11): 2056-61, the contents of which are hereby incorporated herein by reference in their entireties for all purposes.

Other examples of medical conditions include, but are not limited to: tissue viability (e.g., whether tissue is dead or living, and/or whether it is predicted to remain living); tissue ischemia; malignant cells or tissues (e.g., delincating malignant from benign tumors, dysplasias, precancerous tissue, metastasis); tissue infection and/or inflammation; and/or the presence of pathogens (e.g., bacterial or viral counts). Some embodiments include differentiating different types of tissue from each other, for example, differentiating bone from flesh, skin, and/or vasculature. Some embodiments exclude the characterization of vasculature.

In yet other embodiments, the systems, methods and devices provided herein can be used during surgery, for example to determine surgical margins, evaluate the appropriateness of surgical margins before or after a resection, evaluate or monitor tissue viability in near-real time or real-time, or to assist in image-guided surgery. For more information on the use of hyperspectral imaging during surgery, see, Holzer et al., J Urol. 2011 August; 186 (2): 400-4; Gibbs-Strauss et al., Mol Imaging. 2011 April; 10 (2): 91-101; and Panasyuk et al., Cancer Biol Ther. 2007 March; 6 (3): 439-46, the contents of which are hereby incorporated herein by reference in their entirety for all purposes.

For more information on the use of hyperspectral imaging in medical assessments, see, for example: Chin et al., J Vasc Surg. 2011 December; 54 (6): 1679-88; Khaodhiar et al., Diabetes Care 2007; 30:903-910; Zuzak et al., Anal Chem. 2002 May 1; 74 (9): 2021-8; Uhr et al., Transl Res. 2012 May; 159 (5): 366-75; Chin et al., J Biomed Opt. 2012 February; 17 (2): 026010; Liu et al., Sensors (Basel). 2012; 12 (1): 162-74; Zuzak et al., Anal Chem. 2011 Oct. 1; 83 (19): 7424-30; Palmer et al., J Biomed Opt. 2010 November-December; 15 (6): 066021; Jafari-Saraf and Gordon, Ann Vasc Surg. 2010 August; 24 (6): 741-6; Akbari et al., IEEE Trans Biomed Eng. 2010 August; 57 (8): 2011-7; Akbari et al., Conf Proc IEEE Eng Med Biol Soc. 2009:1461-4; Akbari et al., Conf Proc IEEE Eng Med Biol Soc. 2008:1238-41; Chang et al., Clin Cancer Res. 2008 Jul. 1; 14 (13): 4146-53; Siddiqi et al., Cancer. 2008 Feb. 25; 114 (1): 13-21; Liu et al., Appl Opt. 2007 Dec. 1; 46 (34): 8328-34; Zhi et al., Comput Med Imaging Graph. 2007 December; 31 (8): 672-8; Khaodhiar et al., Diabetes Care. 2007 April; 30 (4): 903-10; Ferris et al., J Low Genit Tract Dis. 2001 April; 5 (2): 65-72; Greenman et al., Lancet. 2005 Nov. 12; 366 (9498): 1711-7; Sorg et al., J Biomed Opt. 2005 July-August; 10 (4): 44004; Gillies et al., and Diabetes Technol Ther. 2003; 5 (5): 847-55, the contents of which are hereby incorporated herein by reference in their entirety for all purposes.

In yet other embodiments, the systems, methods and devices provided herein can be used during surgery, for example to determine surgical margins, evaluate the appropriateness of surgical margins before or after a resection, evaluate or monitor tissue viability in near-real time or real-time, or to assist in image-guided surgery. For more information on the use of hyperspectral imaging during surgery, see, Holzer et al., J Urol. 2011 August; 186 (2): 400-4; Gibbs-Strauss et al., Mol Imaging. 2011 April; 10 (2): 91-101; and Panasyuk et al., Cancer Biol Ther. 2007 March; 6 (3): 439-46, the contents of which are hereby incorporated herein by reference in their entirety for all purposes.

Hyperspectral and multispectral imaging are related techniques in larger class of spectroscopy commonly referred to as spectral imaging or spectral analysis. Typically, hyperspectral imaging relates to the acquisition of a plurality of images, each image representing a narrow spectral band collected over a continuous spectral range, for example, 5 or more (e.g., 5, 10, 15, 20, 25, 30, 40, 50, or more) spectral bands having a FWHM bandwidth of 1 nm or more each (e.g., 1 nm, 2 nm, 3 nm, 4 nm, 5 nm, 10 nm, 20 nm or more), covering a contiguous spectral range (e.g., from 400 nm to 800 nm). In contrast, multispectral imaging relates to the acquisition of a plurality of images, each image representing a narrow spectral band collected over a discontinuous spectral range.

For the purposes of the present disclosure, the terms “hyperspectral” and “multispectral” are used interchangeably and refer to a plurality of images, each image representing a narrow spectral band (e.g., having a FWHM bandwidth of between 10 nm and 30 nm, between 5 nm and 15 nm, between 5 nm and 50 nm, less than 100 nm, between 1 and 100 nm, etc.), whether collected over a continuous or discontinuous spectral range.

The terminology used herein is for the purpose of describing particular embodiments only and is not intended to be limiting of the claims. As used in the description of the embodiments and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

It will also be understood that, although the terms “first,” “second,” etc. may be used herein to describe various elements, these elements should not be limited by these terms. These terms are only used to distinguish one element from another. For example, a first spectral range could be termed a second spectral range, and, similarly, a second spectral range could be termed a first spectral range, which changing the meaning of the description, so long as all occurrences of the first spectral range are renamed consistently and all occurrences of the second spectral range are renamed consistently. The first spectral range and the second spectral range are both spectral ranges, but they are not the same spectral range.

As used herein, the term “if” may be construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” may be construed to mean “upon determining” or “in response to determining” or “in accordance with a determination” or “upon detecting” or “in response to detecting” that the stated condition precedent is true, depending on the context.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the invention and its practical applications, to thereby enable others skilled in the art to best utilize the invention and various embodiments with various modifications as are suited to the particular use contemplated.

What is claimed is:

1. A device comprising:
a chassis comprising a base face, wherein an axis is orthogonal to the base face and runs through the base face, and wherein the chassis further comprises:
an inner perimeter wall on the base face and surrounding the axis, the inner perimeter wall enclosing an interior region of the chassis, and
one or more outer walls on the base face, wherein the one or more outer walls are arranged around the inner perimeter wall, wherein each respective outer wall in the one or more outer walls has a respective inward face facing the inner perimeter wall and a respective outward face opposing the respective inward face, and wherein the respective outward face of each of the one or more outer walls is extended at a corresponding acute angle with respect to the base face;
one or more light sources, wherein each light source in the one or more light sources (i) is attached to or disposed on the respective inward face of a corresponding outer wall in the one or more outer walls and (ii) is in optical communication with a region of interest (ROI);
a lens attached to or disposed within the chassis, wherein the lens is in optical communication with light backscattered by the ROI through the interior region of the chassis;
an optical filter comprising a plurality of optical filter elements in optical communication with the lens, wherein each respective optical filter element in the plurality of optical filter elements is characterized by a corresponding spectral band-pass range in a plurality of spectral band-pass ranges;
an optical detector attached to or disposed within the chassis, in optical communication with the optical filter, wherein the optical detector comprises a plurality of optical detector elements arranged in a two-dimensional array, wherein each respective optical detector element in the plurality of optical detector elements is covered by a corresponding optical filter element in the plurality of optical filter elements; and
a control system in electrical communication with the one or more light sources and the optical detector, wherein the control system encodes instructions for the reading the plurality of optical detector elements and for powering the one or more light sources between a powered state and an unpowered state,
wherein the lens is surrounded by the inner perimeter wall, and the inner perimeter wall has a height that blocks light from the one or more light source from reaching the lens.

2. The device of claim 1, wherein the control system comprises:
one or more processors;
memory; and
one or more programs stored in the memory and configured to be executed by the one or more processors, wherein the one or more programs include instructions for:
turning on the one or more light sources; and
using the optical detector to collect light, thereby generating a plurality of detector outputs.

3. The device of claim 2, wherein the one or more programs stored in the memory further include instructions for performing a spectral analysis on the plurality of detector outputs to determine a concentration value of each respective spectral signature in one or more spectral signatures associated with the ROI.

4. The device of any one of claims 1-3, wherein the optical detector comprises one or more photodiodes, a charge-coupled device (CCD), a complementary metal-oxide-semiconductor (CMOS), an active-pixel sensor (APS), or any combination thereof.

5. The device of claim 4, wherein each respective optical detector element in the plurality of optical detector elements comprises a single photodiode.

6. The device of claim 4, wherein each respective optical detector element in the plurality of optical detector elements comprises a single pixel or a cluster of pixels of the CCD, CMOS or APS.

7. The device of claim 1, wherein the one or more light sources is a plurality of light sources wherein the control system comprises:
one or more processors;
memory; and
one or more programs stored in the memory and configured to be executed by the one or more processors, wherein the one or more programs encode the instructions for the reading the plurality of optical detector elements and for powering the plurality of light sources between a powered state and an unpowered state by:
sequentially turning on and off each respective light source in the plurality of light source sequentially; and
sequentially reading the plurality of optical detector elements when each respective light source is turned on, thereby generating a plurality of arrays of detector outputs, each array of detector outputs at a corresponding band-pass range in the plurality of band-pass ranges, wherein the plurality of arrays of detector outputs collectively forms a hyperspectral data cube of detector outputs.

8. The device of claim 13, wherein the one or more programs stored in the memory further include instructions for performing a spectral analysis on the hyperspectral data cube of detector outputs to determine concentration values of each respective spectral signature in one or more spectral signatures at each respective point in an array of points corresponding to a two-dimensional area of the ROI.

9. The device of claim 3 or 8, wherein a spectral signature in the one or more spectral signatures is oxyhemoglobin, deoxyhemoglobin, or melanin.

10. The device of claim 9, wherein an oxygen saturation value or an oximetry index value is determined based on the concentration values of the one or more spectral signatures.

11. The device of any one of claims 1-10, wherein the interior region of the chassis surrounded by the inner perimeter wall has diameter that is between 5 mm and 10 mm, between 10 and 15 mm, or between 15 and 20 mm.

12. The device of claim 11, wherein the interior region of the chassis surrounded by the inner perimeter wall is circular, oval, or has an n-gon cross-section, wherein n is a positive integer greater than 3.

13. The device of any one of claims 1-12, wherein a height of the inner perimeter wall is less than 10 mm, less than 8 mm, less than 6 mm, or less than 4 mm.

14. The device of any one of claims 1-13, wherein the corresponding acute angle is between 15 degrees and 30 degrees, 30 degrees, between 30 degrees and 45 degrees, 45 degrees, between 45 degrees and 60 degrees, 60 degrees, or between 60 degrees and 75 degrees.

15. The device of any one of claims 1-14, wherein the one or more outer walls collectively form a truncated conical structure on the base face, wherein
a base of the truncated conical structure at the base face is between 10 mm and 15 mm, between 15 mm and 20 mm, between 20 mm and 25 mm, or between 25 mm and 30 mm; and
a diameter at the top of the truncated conical structure is between 15 mm and 25 mm, between 25 mm and 35 mm, or between 35 and 45 mm.

16. The device of claim 15, wherein the truncated conical structure has a circular, oval, or n-gon cross-section, wherein n is a positive integer greater than 5.

17. The device of any one of claims 1-16, wherein each outer wall in the one or more outer walls has a height relative to the base face that is less than 14 mm, less than 12 mm, less than 10 mm, less than 8 mm, or less than 6 mm.

18. The device of any one of claims 1-17, wherein the one or more light sources comprises three, four, five, six, seven, eight, or more than eight light source elements.

19. The device of claim 18, wherein the three, four, five, six, seven, eight or more than eight light source elements are uniformly distributed around the interior region.

20. The device of any one of claims 1-19, wherein each light source in the one or more light sources emits near infrared light, visible light, ultraviolet light, or any combination thereof when in the powered state.

21. The device of any one of claims 1-20, wherein each light source in the one or more light sources emits white light between 400 nm and 780 nm.

22. The device of any one of claims 1-21, wherein the ROI has a size less than 700 mm², less than 650 mm², less than 600 mm², less than 550 mm², less than 500 mm², less than 450 mm², less than 400 mm², less than 350 mm², less than 300 mm², less than 250 mm², less than 200 mm², less than 150 mm², less than 100 mm², or less than 50 mm².

23. The device of any one of claims 1-22, wherein the ROI is located less than 50 mm, less than 40 mm, less than 30 mm, less than 25 mm, less than 20 mm, 18 mm, less than 16 mm, less than 14 mm, less than 12 mm, less than 10 mm, less than 8 mm, less than 6 mm, less than 4 mm, or less than 3 mm from the base face.

24. The device of any one of claims 1-23, wherein each respective optical filter element in the plurality of optical filter elements is a bandpass filter.

25. The device of claim 24, wherein each respective optical filter element in the plurality of optical filter elements has a different band-pass range than any other optical filter element in the plurality of optical filter elements.

26. The device of claim 24, wherein at least two optical filter elements in the plurality of optical filter elements have a common band-pass range.

27. The device of any one claims 1-26, further comprising:
a first polarizer comprising one or more first polarizer elements, wherein the first polarizer is disposed in an optical path between the light source and the lens, and is configured to selectively allow light that is substantially limited to a first polarization to pass through the lens; and
a second polarizer comprising a plurality of second polarizer elements, wherein the second polarizer is disposed in an optical path between the lens and the optical detector, and is configured to selectively allow light that is substantially limited to a second polarization to pass to the optical detector, wherein the second polarization is in a different direction from the first polarization.

28. The device of any one of claims 1-27, further comprising:
a power source in electrical communication with the light source, the optical detector, and/or the control system to provide electrical power to the one or more light sources, the optical detector, and/or the control system.

29. The device of claim 28, wherein the power source comprises one or more batteries.

30. The device of claim 28, wherein the power source has a voltage of 10 volts or less.

31. The device of claim 29, wherein a battery in the one or more batteries comprises a lithium button cell and/or a lithium polymer battery.

32. The device of any one of claims 28-31, wherein the control system further comprises a power regulator that maintains a power supply at a desired level.

33. The device of any one of claims 1-32, wherein the control system further comprises a communication interface in wired or wireless communication with an external device or communication network.

34. The device of claim 33, wherein the detector outputs are communicated to the external device or communication network, and analysis of the detector outputs is performed at the external device or communication network.

35. The device of any one of claims 1-34, further comprising:
a casing for housing the chassis and the control system, wherein the base face faces an exterior of the casing.

36. The device of claim 35, wherein the casing is configured to be fitted into different enclosures.

37. The device of claim 36, wherein the casing is sleevable between the ROI and a wrapper, and able to be snapped-fitted into a housing comprising a gripping knob.

38. The device of any one of claims 1-37, further comprises an internal casing display, mounted casing display, or external display.

39. The device of any one of claims 1-38, wherein the control system comprises a mobile application.

40. The device of any one of claims 1-39, wherein the lens is disposed within or over the interior region of the chassis and fixed to the inner perimeter wall.

41. The device of any one of claims 1-40, wherein the optical filter is disposed within the interior region of the chassis and fixed to the inner perimeter wall.

42. The device of any one of claims 1-40, wherein the optical detector is disposed within the interior region of the chassis and fixed to the inner perimeter wall.

43. A method performed at the device of any one of claims 1-42.

44. A non-transitory computer-readable storage medium storing one or more programs, the one or more programs comprising instructions to perform the method of claim 43.

45. A system, comprising:
the device of any one of the claims 1-44;
a first client device in a wireless communication with the device; and
a server in a wireless communication with the device and the first client device, the server including one or more central processing units, memory, and one or more programs, wherein the one or more programs are stored in the memory and are configured to be executed by the one or more central processing units, the one or more programs including instructions for:
(A) receiving, from the device, the hyperspectral data cube of detector outputs;
(B) forming a hyperspectral image using the hyperspectral data cube of detector outputs; and
(C) transmitting, to the first client device, the hyperspectral image.

46. The system of claim 45, wherein:
forming the hyperspectral image includes determining the oxygen saturation value or the oximetry index value based on the concentration values of the one or more spectral signatures in the hyperspectral image, and
transmitting the hyperspectral image to the first client device includes transmitting the oxygen saturation value or the oximetry index value.

47. The system of claim 45, wherein the first client device is a mobile device operated by a patient, the system thereby enabling the patient to self-monitor a condition associated with the oxygen saturation value or the oximetry index value.
